(19) United States
(12) Patent Application Publication
(10) Pub. No.: US 2025/0232872 A1
(43) Pub. Date: Jul. 17, 2025

(54) Assistant System Using Multimodal Multitask Medical Machine-Learned Models to Perform Image Processing to Answer Natural Language Queries

(71) Applicant: Google LLC, Mountain View, CA (US)

(72) Inventors: Vivek Natarajan, Sunnyvale, CA (US); Shekoofeh Azizi, Vancouver (CA); Alan Prasana Karthikesalingam, London (GB); Danny Michael Driess, Berlin (DE); Peter Raymond Florence, San Francisco, CA (US); Karan Singhal, Roslyn, NY (US); Tao Tu, New York, NY (US)

(21) Appl. No.: 18/411,102

(22) Filed: Jan. 12, 2024

(51) Int. Cl. G16H 50/20; G16H 15/00; G16H 20/00; G16H 30/40; G16H 40/67

(52) U.S. Cl. CPC G16H 50/20; G16H 15/00; G16H 20/00; G16H 30/40; G16H 40/67

(57) ABSTRACT

An example assistant system can use a multimodal multitask medical machine-learned model to perform image processing to answer natural language queries. A device can process speech data or other natural language inputs to obtain a query. The query can be processed alongside image data that provides context for the query. The example system can receive a query associated with a particular task domain; generate, based on the query, a query input that comprises query instruction data from a first modality and query context data from a second modality; generate a combined input comprising the query input and an exemplar input, wherein the exemplar input comprises exemplar instruction data from the first modality and an exemplar context placeholder in lieu of exemplar context data from the second modality; process the combined input with a multimodal machine-learned model to generate output data; and output a query response based on the output data.

### FIELD

The present disclosure relates generally to machine learning processes and machine-learned devices and systems. More particularly, the present disclosure relates to data efficient techniques for training multitask multimodal machine-learned models.

### BACKGROUND

A computer can receive input(s). The computer can execute instructions to process the input(s) to generate output(s) using a parameterized model. The computer can obtain feedback on its performance in generating the outputs with the model. The computer can generate feedback by evaluating its performance. The computer can receive feedback from an external source. The computer can update parameters of the model based on the feedback to improve its performance. In this manner, the computer can iteratively “learn” to generate the desired outputs. The resulting model is often referred to as a machine-learned model.

### SUMMARY

Aspects and advantages of embodiments of the present disclosure will be set forth in part in the following description, or can be learned from the description, or can be learned through practice of the embodiments.

Example aspects of the present disclosure provide an example computing system that includes one or more processors and one or more example non-transitory computer-readable media storing instructions that are executable by one or more processors to cause a computing system to perform example operations. The example operations can include receiving a query associated with a particular task domain. The example operations can include generating, based on the query, a query input that can include query instruction data from a first modality and query context data from a second modality. The example operations can include generating a combined input that can include the query input and an exemplar input, wherein the exemplar input can include exemplar instruction data from the first modality and an exemplar context placeholder in lieu of exemplar context data from the second modality. The example operations can include processing the combined input with a multimodal machine-learned model to generate output data. The example operations can include outputting a query response based on the output data.

In some implementations of the example computing system, the operations can include generating a score based on the output data.

In some implementations of the example computing system, the operations can include training the multimodal machine-learned model based on the score.

In some implementations of the example computing system, the query input can include the instruction data from the first modality interleaved with the context data from the second modality.

In some implementations of the example computing system, the machine-learned model is a sequence processing model.

In some implementations of the example computing system, the machine-learned model can include one or more transformer layers.

In some implementations of the example computing system, the machine-learned model can include: one or more first modality input layers configured to process data from the first modality and project the data from the first modality into a latent space of the machine-learned model; and one or more second modality input layers configured to process data from the second modality and project the data from the second modality into the latent space.

In some implementations of the example computing system, the operations can include detecting second modality data in the query.

In some implementations of the example computing system, the operations can include, responsive to detecting second modality data in the query, routing the second modality data to a machine-learned sequence encoder configured to process the second modality data and generate a sequence representing the second modality data.

In some implementations of the example computing system, the operations can include, based on detecting the second modality data in the query, selecting the machine-learned sequence encoder from among a plurality of modality-specific machine-learned sequence encoders.

Example aspects of the present disclosure provide an example computing system that includes one or more processors and one or more example non-transitory computer-readable media storing instructions that are executable by one or more processors to cause a computing system to perform example operations. The example operations can include processing a training batch with the multimodal machine-learned model to generate output data. In the example computing system, the training batch can include a plurality of training query inputs that can include, for each respective task domain of a plurality of task domains, a respective set of training query inputs associated with the respective task domain, each training query input in the respective set of training query inputs that can include instruction data in a first modality and context data in a second modality. The example operations can include outputting, based on the output data, training query responses respectively corresponding to the plurality of training query inputs. The example operations can include training the multimodal machine-learned model based on evaluations of the training query responses.

In some implementations of the example computing system, the training batch can include a unimodal set of training query inputs associated with a unimodal task domain, each training query input in the unimodal set of training query inputs that can include instruction data in a first modality and context data in the first modality.

In some implementations of the example computing system, the training batch can include at least four query inputs associated with each respective task domain.

In some implementations of the example computing system, the training batch can include at least one query input associated with two or more of the following task domains: question answering; report summarization; visual question answering; report generation; and image classification.

In some implementations of the example computing system, the training batch can include at least one query input associated with each of the following task domains: question answering; report summarization; visual question answering; report generation; and image classification.

In some implementations of the example computing system, the training batch can include, for the visual question answering task domain: at least one query input associated with a radiology task; and at least one query input associated with a pathology task.

In some implementations of the example computing system, over half the training batch is associated with the report generation task.

In some implementations of the example computing system, the training batch can include a plurality of exemplar inputs respectively associated with the plurality of query inputs, wherein at least one of the plurality of exemplar inputs is unimodal.

In some implementations of the example computing system, the operations can include detecting second modality data in a training query. In some implementations of the example computing system, the operations can include selecting, from among a plurality of modality-specific machine-learned sequence encoders, a machine-learned sequence encoder configured to process the second modality data and generate a sequence representing the second modality data. In some implementations of the example computing system, the operations can include routing the second modality data to the machine-learned sequence encoder for processing.

Example aspects of the present disclosure provide an example computing system that includes a natural language interface associated with a natural language modality. The example computing system can include an image capture interface associated with an image modality. The example computing system can include one or more processors and one or more example non-transitory computer-readable media storing instructions that are executable by one or more processors to cause a computing system to perform example operations. The example operations can include recording natural language data using the natural language interface. The example operations can include recording image data using the image capture interface. The example operations can include generating a query that can include the natural language data and the image data. The example operations can include providing the query to a multimodal machine-learned sequence processing model that generates a query response based on the query. The example operations can include rendering the query response.

In some implementations of the example computing system, the multimodal machine-learned sequence processing model was trained by processing a training batch with the multimodal machine-learned sequence processing model to generate output data, wherein the training batch can include a plurality of training query inputs that can include, for each respective task domain of a plurality of task domains, a respective set of training query inputs associated with the respective task domain, each query input in the respective set of training query inputs that can include instruction data in a first modality and context data in a second modality. In some implementations of the example computing system, the multimodal machine-learned sequence processing model was trained by outputting, based on the output data, training query responses respectively corresponding to the plurality of training query inputs. In some implementations of the example computing system, the multimodal machine-learned sequence processing model was trained by training the multimodal machine-learned model based on evaluations of the training query responses.

In some implementations of the example computing system, the training batch can include at least four training query inputs associated with two or more of the following task domains: question answering; report summarization; visual question answering; report generation; and image classification. In some implementations of the example computing system, over half the training batch is associated with the report generation task.

Example aspects of the present disclosure provide an example computing system that includes a natural language interface associated with a natural language modality. The example computing system can include an image capture interface associated with an image modality. The example computing system can include one or more processors and one or more example non-transitory computer-readable media storing instructions that are executable by one or more processors to cause a computing system to perform example operations. The example operations can include recording natural language data using the natural language interface. The example operations can include recording image data using the image capture interface. The example operations can include generating a query that can include the natural language data and the image data. The example operations can include providing the query to a multimodal machine-learned sequence processing model that generates a query response based on the query. The example operations can include rendering the query response.

In some implementations of the example computing system, the multimodal machine-learned sequence processing model was trained by receiving a training query associated with a particular task domain. In some implementations of the example computing system, the multimodal machine-learned sequence processing model was trained by generating, based on the training query, a training query input that can include training query instruction data from a first modality and training query context data from a second modality. In some implementations of the example computing system, the multimodal machine-learned sequence processing model was trained by generating a combined training input that can include the training query input and a training exemplar input, wherein the training exemplar input can include training exemplar instruction data from the first modality and an exemplar context placeholder in lieu of training exemplar context data from the second modality. In some implementations of the example computing system, the multimodal machine-learned sequence processing model was trained by processing the combined training input with the multimodal machine-learned sequence processing model to generate training output data. In some implementations of the example computing system, the multimodal machine-learned sequence processing model was trained by updating one or more parameters of the machine-learned multimodal sequence processing model based on the training output data.

In some implementations of the example computing system, the example operations include transmitting the query to a server computing system that executes the multimodal machine-learned sequence processing model.

In some implementations of the example computing system, the example operations include transmitting a runtime exemplar to the server computing system, wherein the runtime exemplar can include the exemplar context placeholder in lieu of runtime exemplar context data from the second modality.

In some implementations of the example computing system, the runtime exemplar is customized in association with a user account associated with the computing system.

Other example aspects of the present disclosure are directed to other systems, methods, apparatuses, tangible non-transitory computer-readable media, and devices for performing functions described herein. These and other features, aspects, and advantages of various implementations will become better understood with reference to the following description and appended claims. The accompanying drawings, which are incorporated in and constitute a part of this specification, illustrate implementations of the present disclosure and, together with the description, help explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 is a block diagram of an example system according to example aspects of some embodiments of the present disclosure;

FIG. 2 is a block diagram of an example input structure according to example aspects of some embodiments of the present disclosure;

FIG. 3 is a block diagram of an example input structure according to example aspects of some embodiments of the present disclosure;

FIG. 4 is a block diagram of an example input structure according to example aspects of some embodiments of the present disclosure;

FIG. 5 is a block diagram of an example input structure according to example aspects of some embodiments of the present disclosure;

FIG. 6 is a block diagram of an example system according to example aspects of some embodiments of the present disclosure;

FIG. 7 is a block diagram of an example system according to example aspects of some embodiments of the present disclosure;

FIG. 8A is a block diagram of an example system according to example aspects of some embodiments of the present disclosure;

FIG. 8B is a block diagram of an example system according to example aspects of some embodiments of the present disclosure;

FIG. 9 is a block diagram of an example system according to example aspects of some embodiments of the present disclosure;

FIG. 10 is a block diagram of an example user interface for a system according to example aspects of some embodiments of the present disclosure;

FIG. 11 is a block diagram of an example user interface for a system according to example aspects of some embodiments of the present disclosure;

FIG. 12 is a flow chart diagram illustrating an example method for implementing a machine-learned model according to example implementations of aspects of the present disclosure;

FIG. 13 is a flow chart diagram illustrating an example method for training a machine-learned model according to example implementations of aspects of the present disclosure;

FIG. 14 is a flow chart diagram illustrating an example method for implementing a machine-learned model according to example implementations of aspects of the present disclosure;

FIG. 15 is a flow chart diagram illustrating an example method for training a machine-learned model according to example implementations of aspects of the present disclosure;

FIG. 16 is a block diagram of an example processing flow for using machine-learned model(s) to process input(s) to generate output(s) according to example implementations of aspects of the present disclosure;

FIG. 17 is a block diagram of an example sequence processing model according to example implementations of aspects of the present disclosure;

FIG. 18 is a block diagram of an example technique for populating an example input sequence for processing by a sequence processing model according to example implementations of aspects of the present disclosure;

FIG. 19 is a block diagram of an example model development platform according to example implementations of aspects of the present disclosure;

FIG. 20 is a block diagram of an example training workflow for training a machine-learned model according to example implementations of aspects of the present disclosure;

FIG. 21 is a block diagram of an inference system for operating one or more machine-learned model(s) to perform inference according to example implementations of aspects of the present disclosure;

FIG. 22 is a block diagram of an example networked computing system according to example implementations of aspects of the present disclosure;

FIG. 23 is a block diagram of an example computing device according to example implementations of aspects of the present disclosure; and

FIG. 24 is a block diagram of an example computing device according to example implementations of aspects of the present disclosure.

Reference numerals that are repeated across plural figures are intended to identify various implementations of the same features.

### DETAILED DESCRIPTION

### Overview

Example aspects of the present disclosure generally relate to using and adapting machine-learned models to perform tasks in multiple specialized knowledge domains. In various implementations, example systems of the present disclosure can implement a multimodal machine-learned model. The multimodal machine-learned model can be a foundational model that can use a shared set of model weights to achieve benchmark performance on diverse tasks from multiple different specialized knowledge domains. In this manner, for instance, a single model can be trained and distributed to various systems to perform different tasks, increasing efficiency and simplicity of integrating and maintaining machine-learned components in various workflows.

Example models can be used to implement assistant systems that can assist users in performing various different tasks. An assistant system can receive query data related to a task and generate responses. The responses can answer a question, provide additional information, or initiate performance of further processing tasks related to the query. The assistant system can interact with users using various input and output modalities. An example assistant system can provide a conversational speech interface for interacting with a user. Other interfaces (e.g., text, video, haptic, audio) can be used.

Example implementations of the present disclosure can leverage improved one- or few-shot prompting techniques. For example, one or more exemplars can be included in an input to the multimodal machine-learned model. The exemplar(s) can demonstrate a desired performance from the multimodal machine-learned model. For instance, for a report generation task for a radiology image input, an exemplar can include an example report generated based on a radiology image. However, inputting additional images as exemplars can increase a size of an input. And some models might inappropriately cross-attend between an exemplar image and the actual query, confusing the present task with the example task.

Example implementations of the present disclosure can advantageously omit one or more modalities from an exemplar input to generate a lightweight few-shot input. For example, to continue the above example, the exemplar radiology input can include the example report but omit the exemplar radiology image. In this manner, for instance, the exemplar input can act as a schema, demonstrating the correct structure, content, and style of a radiology report, without introducing undue processing overhead or introducing avoidable confusion of inputs. To maintain the pattern of input between the exemplar and the runtime query, example implementations can process a placeholder in the exemplar input in lieu of processing the exemplar image. For example, an image can be replaced by a textual tag, such as “<img>.”

This substitution can be implemented in training (e.g., fine-tuning) and at inference time. In this manner, for example, the model can efficiently learn from multimodal datasets, even without processing one or more of the multimodal exemplars.

Example implementations of the present disclosure can provide for improved multi-task training techniques. For instance, example machine-learned models trained according to the present disclosure can achieve benchmark performance across multiple different tasks. By training a model to perform multiple tasks, the model can learn knowledge from each task and apply that knowledge when executing other tasks. For instance, training a generalist biomedical AI system with language as a common grounding across different tasks allows the system to tackle new tasks by combining the knowledge it has learned for other tasks (i.e. combinatorial generalization). Example results of the present disclosure indicate that example implementations can generalize to novel medical concepts and unseen tasks in a zero-shot fashion.

For example, the zero-shot generalization capability of an example implementation of the present disclosure for an unseen medical concept by evaluating its ability to detect tuberculosis (TB) abnormality from chest X-ray images in the Montgomery County (MC) dataset. The example implementation of the present disclosure performed competitively compared to state of the art results obtained by a specialized ensemble model optimized for this dataset. The state-of-the-art, specialized ensemble model that was optimized for this exact task reached 92.60% accuracy. The example implementation of the present disclosure, a generalized system that had no exposure to labeled TB images, reached 87.68% accuracy. Similar performance was observed across three model variants, consistent with findings on other medical image classification tasks in MultiMedBench.

One technique of example aspects of the present disclosure that can improve multi-task performance is enforce a task diversity metric within training batches used to train the multimodal machine-learned model. For example, a task diversity metric can include one or more minimum thresholds for numbers of examples in the training batch. For instance, a training batch can include multiple inputs. The multiple inputs can be drawn from multiple different task domains. The multiple inputs can include multiple examples from each different task domain. In this manner, for example, improved multi-task and cross-task generalization performance can be achieved.

Example implementations of the present disclosure can provide for improved multimodal task assistant devices for specialized task domains. For instance, a mobile device or wearable device can record natural language input data and record image data. The device can generate multimodal inputs to query a multimodal machine-learned model. The device can receive and render the query response. Such a device can be used in a medical setting as a medical assistant device. Such a device can be used by medical professionals or by patients to answer questions, identify treatment plans, create checklists, generate or populate documents or document templates, etc. Example multimodal, multi-task models according to the present disclosure can enable device(s) to use or interact with one model that can leverage cross-task knowledge to assist in complex, mixed-domain real world environments.

Example implementations can provide advances in areas of medical tasks. Medicine is generally a multimodal discipline. Clinicians routinely interpret data from a wide range of modalities including clinical notes, laboratory tests, vital signs and observations, medical images, genomics, and more when providing care. Despite significant progress in biomedical AI, most models today are unimodal single task systems. Some existing systems obtain state-of-the-art (SOTA) performance on breast cancer screening but cannot incorporate relevant information such as patient health records (e.g., breast cancer gene screening status), other modalities such as MRI, or published medical literature that might help contextualize, refine, and improve performance. Further, the system's output is constrained to a pre-specified set of possible classifications. It cannot verbally explain its prediction or engage in a collaborative dialogue to learn from a physician's feedback. This bounds performance and utility of these narrow, single-task, unimodal, specialist AI systems in real-world applications.

In contrast, example implementations of the present disclosure can provide a generalist biomedical AI system that can handle a diverse range of biomedical data modalities and tasks. This can be accomplished using a single set of model weights, without any task-specific customization.

Example implementations of the present disclosure can provide a number of technical effects and benefits. A technical effect of example implementations of the present disclosure is increased data efficiency for adapting machine-learned models to particular tasks. For instance, techniques according to the present disclosure can enable significant improvements in model performance using relatively small amounts of reference data. In this manner, costs of obtaining reference or training data can be reduced or otherwise mitigated. Less training data can correspond to less storage and compute for obtaining, preparing, and storing that data. Less training data can also correspond to decreased numbers of training iterations.

A technical effect of example implementations of the present disclosure is increased accuracy and performance of computing systems that implement models adapted according to the present disclosure. For instance, due to the data efficiency of the example implementations of the disclosed techniques, computing systems can execute machine-learned models to perform tasks in specialized knowledge domains, when such models would otherwise be cost-prohibitive to train to a requisite threshold performance according to traditional techniques. As such, the presently disclosed techniques can unlock previously unrealizable functionality of computing systems and devices.

A technical effect of example implementations of the present disclosure is improved data security and privacy. For example, various implementations of the present disclosure adapt and align pre-trained models to new knowledge domains without changing the parameters of the model itself. Instead, such implementations adapt a model input to better instruct the model to perform tasks in that specialized domain. In this manner, for instance, any sensitive information that could be used to adapt the model (if appropriate permissions were provided) could remain in the input to the model and not be in any way retained within the model parameters itself. In this manner, for instance, personalized prompt structures can be built and maintained by a user. Since the prompt structure(s) can be smaller than the model itself, then it can be easier for the user to maintain the prompt structure(s) in a secure storage environment (e.g., on a secured device, in a secured online account, etc.).

A technical effect of example implementations of the present disclosure is increased energy efficiency in performing operations using machine-learned models, thereby improving the functioning of computers implementing such models. For instance, example implementations can provide for more energy-efficient runtime execution or inference by using lightweight inputs. In some scenarios, increased energy efficiency can provide for less energy to be used to perform a given task (e.g., less energy expended to maintain the model in memory, less energy expended to perform calculations within the model, etc.). In some scenarios, increased energy efficiency can provide for more task(s) to be completed for a given energy budget (e.g., a larger quantity of tasks, more complex tasks, the same task but with more accuracy or precision, etc.).

In another example aspect, example implementations can provide for more energy-efficient training operations or model updates by using lightweight inputs or by using highly effective training batch mixtures. In some scenarios, increased energy efficiency can provide for less energy to be used to perform a given number of update iterations (e.g., less energy expended to maintain the model in memory, less energy expended to perform calculations within the model, such as computing gradients, backpropagating a loss, etc.). In some scenarios, increased energy efficiency can provide for more update iterations to be completed for a given energy budget (e.g., a larger quantity of iterations, etc.). In some scenarios, greater expressivity afforded by model architectures and training techniques of the present disclosure can provide for a given level of functionality to be obtained in fewer training iterations, thereby expending a smaller energy budget. In some scenarios, greater expressivity afforded by model architectures and training techniques of the present disclosure can provide for an extended level of functionality to be obtained in a given number of training iterations, thereby more efficiently using a given energy budget.

In this manner, for instance, the improved energy efficiency of example implementations of the present disclosure can reduce an amount of pollution or other waste associated with implementing machine-learned models and systems, thereby advancing the field of machine-learning and artificial intelligence as a whole. The amount of pollution can be reduced in toto (e.g., an absolute magnitude thereof) or on a normalized basis (e.g., energy per task, per model size, etc.). For example, an amount of CO2 released (e.g., by a power source) in association with training and execution of machine-learned models can be reduced by implementing more energy-efficient training or inference operations. An amount of heat pollution in an environment (e.g., by the processors/storage locations) can be reduced by implementing more energy-efficient training or inference operations.

Reference now is made to the figures, which provide example arrangements of computing systems, model structures, and data flows for illustration purposes only.

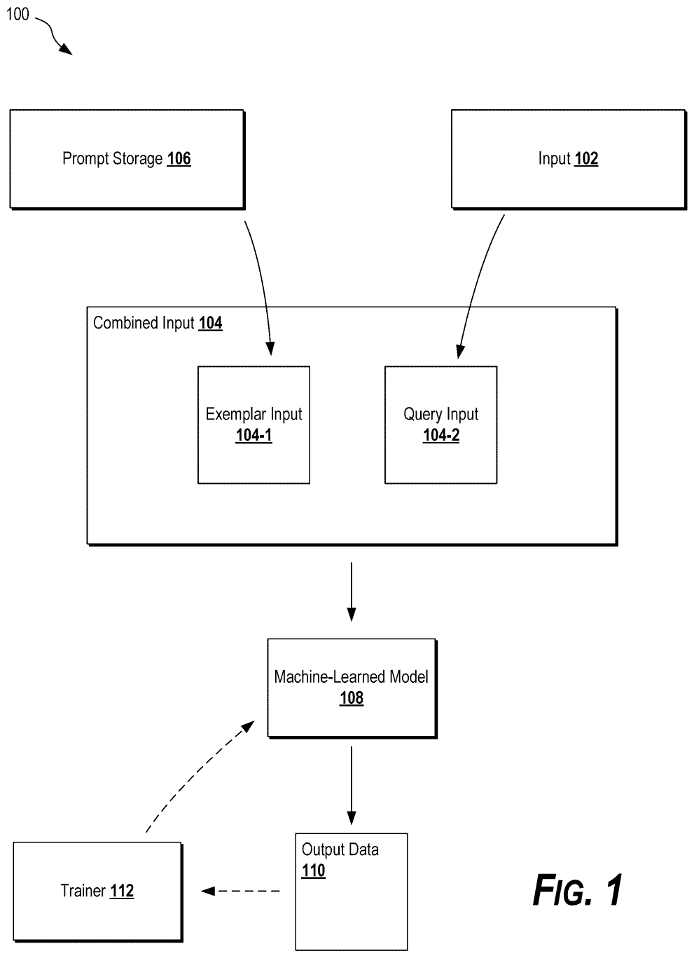

FIG. 1 illustrates an example machine-learned model system 100 according to the present disclosure. The system 100 can be configured for servicing queries (e.g., queries from or for one or more users). The system 100 can obtain input 102. The system 100 can build a combined input sequence 104 using input 102 and one or more values from a prompt storage 106. For instance, exemplar prompt values 104-1 can be obtained from or based on data from prompt storage 106. A query input 104-2 based on input 102 can be combined with exemplar input 104-1. Query input 104-2 can be related to a runtime task and exemplar input 104-1 can be an example of how such a task can be performed. A machine-learned model 108 can process combined input 104 to generate output data 110. In a backwards pass (e.g., dashed lines), a model trainer 112 can evaluate output data 110 and update one or more learned parameters of machine-learned model 108.

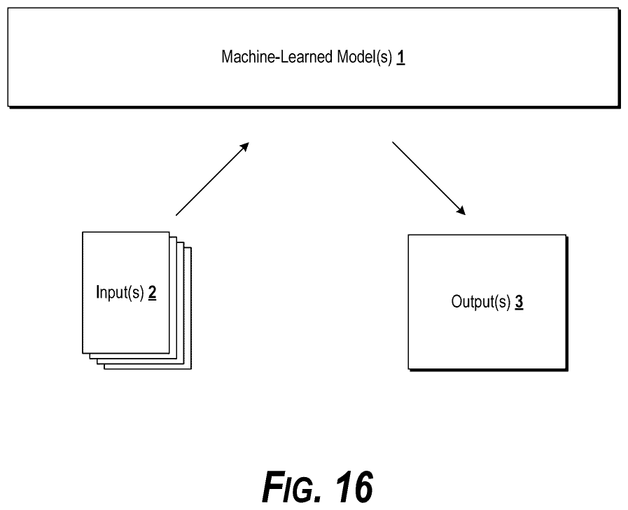

Machine-learned model system 100 can include one or more computing devices or systems. Any or all of the various components illustrated in FIG. 1 can be implemented on separate devices or systems in communication with one another. Example such devices and systems are described in more detail with respect to FIGS. 16-24.

Machine-learned model system 100 can be implemented as a service to provide responses to queries. The service can be a web service. Features of machine-learned model system 100 can be directly accessed by users through a first-party interface (e.g., a user-facing web interface associated with the machine-learned model system 100). Features of machine-learned model system 100 can be accessed by other services or programs through API calls, such that interactions with or results from machine-learned model system 100 can be integrated into third-party systems or interfaces.

Input 102 can include data associated with a query. A query can generally include an input provided to the system 100 for obtaining a response thereto. Input 102 can include a question, a command, context material for a question or command, etc. Input 102 can include natural language content. Input 102 can include one modality of data (e.g., symbolic or text data, audio data, image data, etc.). Input 102 can include multiple modalities of data. For instance, input 102 can include symbolic data and image data. Input 102 can include text data and audio data. Input 102 can include audio data and image data (e.g., video data). Input 102 can include audio data and image data and text data (e.g., video data and associated description or instructions).

Input 102 can be received in one or multiple portions. Contents of input 102 can be sent at one time or streamed over time. Input 102 can correspond to input data obtained over the duration of a communication session with a sender (e.g., a sending device).

Combined input 104 can be or include a data structure configured for processing by machine-learned model 108. The data structure can be configured based on the input dimensions or constraints of machine-learned model 108. Different machine-learned models may have different input dimensions or constraints, and input sequence 104 can be constructed accordingly. For models 108 configured to process input values embedded in a multidimensional latent space, for example, combined input 104 can include a series of tokens representing information embedded in the latent space. Combined input 104 can include a natural language string that can be processed by one or more tokenizers to generate the series of tokens representing information embedded in the latent space.

Combined input 104 can include prompt values. Prompt values can represent prompt data (e.g., context data, instructions, examples) useful to machine-learned model 108 when processing data from input 102. For instance, prompt data can provide example input-output pairs to illustrate how machine-learned model 108 is to respond to inputs. These examples may provide additional explicit or implicit contextual cues to machine-learned model 108 to aid machine-learned model 108 in understanding how to process data from input 102.

Prompt values can represent explicit context. Prompt data can include context described in natural language. In this manner, for instance, prompt values based on the prompt text can include embedding values determined from the natural language content. For instance, the natural language content can be tokenized and embedded into a latent space to represent, numerically, the meaning of the natural language content using the prompt values.

Exemplar inputs 104-1 can include prompt data or prompt values obtained or otherwise derived from natural language content. Exemplar inputs 104-1 can be, can include, or can be embedded to obtain embedding values representing natural language content embedded in a latent space. For instance, a natural language prompt can be tokenized and embedded into exemplar inputs 104-1.

Exemplar inputs 104-1 can represent example input-output pairs. The example inputs can include queries, commands, contextual information, instructions, questions, etc. The example outputs can include answers to questions, responses to commands and instructions, processed contextual information, and contextual information generated in association with answers to questions or responses to commands and instruction (e.g., a rationale for an answer), etc.

Exemplar inputs 104-1 can represent data of various modalities. Exemplar inputs 104-1 can represent one modality of data (e.g., symbolic or text data, audio data, image data, etc.). Exemplar inputs 104-1 can represent multiple modalities of data. For instance, exemplar inputs 104-1 can represent symbolic data and image data (e.g., images and associated descriptions or instructions). Exemplar inputs 104-1 can represent text data and audio data (e.g., audio tracks and associated descriptions or instructions). Exemplar inputs 104-1 can represent audio data and image data (e.g., video data). Exemplar inputs 104-1 can represent audio data and image data and text data (e.g., video data and associated description or instructions).

Exemplar inputs 104-1 can provide domain-related input data to assist a downstream machine-learned model (e.g., model 108) to process input 102 (e.g., via input query values 104-3). Exemplar inputs 104-1 can relate to input 102 in a hierarchical manner. For instance, Exemplar inputs 104-1 can be associated with a domain of tasks or subject matter of the input 102.

Prompt storage 106 can provide exemplar inputs 104-1. Prompt storage 106 can store multiple different sets of exemplar inputs 104-1. Prompt storage 106 can be maintained in volatile or non-volatile memory in any suitable data format. Prompt storage 106 can be implemented using a database, cache, file server, etc.

Query input 104-2 can include values obtained or otherwise derived from natural language content (e.g., natural language content of input 102). Query input 104-2 can be, can include, or can be embedded to obtain embedding values representing natural language content embedded in a latent space. For instance, a natural language input (e.g., input 102) can be tokenized and embedded into query inputs 104-2. Query inputs 104-2 can have the same or different dimensions as exemplar inputs 104-1.

Machine-learned model(s) 108 can be or can otherwise include various machine-learned models such as neural networks (e.g., deep neural networks) or other types of machine-learned models, including non-linear models or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models).

Machine-learned model(s) 108 can be pre-trained on various tasks. Machine-learned model(s) 108 can be pre-trained on a large corpus of training data in a self-supervised manner (e.g., text, audio, or image denoising). Machine-learned model(s) 108 can be fine-tuned on various tasks. Machine-learned model(s) 108 can be fine-tuned using supervised learning or reinforcement learning or other techniques using a smaller corpus of higher quality training data relevant to different kinds of tasks. For instance, machine-learned model(s) 108 can be trained using human feedback. An example machine-learned model is described in Driess et al., PaLM-E: An Embodied Multimodal Language Model (Mar. 6, 2023) arXiv: 2303.03378v1, which is hereby incorporated by reference herein in its entirety.

Machine-learned model(s) 108 can process one modality or multiple modalities. Machine-learned model(s) 108 can process symbolic sequences (e.g., text, computer code, genetic data, etc.), image-based sequences (e.g., tokenized images), audio-based sequences (e.g., tokenized audio), or combinations thereof. Machine-learned model(s) 108 can include model architectures generally referred to as “large language models” or “LLMs.”

Machine-learned model(s) 108 can include one or multiple models. For instance, multiple models can be used to service a query. For instance, an input 102 can include a request for a task to be performed. Multiple models may be used to perform the task. For instance, a first machine-learned model can be used to process combined input 104 to understand the query. A second machine-learned model can be used to perform a sub-task of the query that is associated with the second machine-learned model. For instance, image processing sub-tasks can be passed to a separate image-processing model (e.g., an image generation model, a classification model, or other analysis model). Machine-learned model(s) 108 can include an orchestrator or supervisor model that processes combined input 104 and determines what other model(s) to invoke for responding to the query.

Output data 110 can include an output of machine-learned model(s) 108 responsive to combined input 104. Output data 110 can include a request for additional inputs. For instance, output data 110 can include requests for clarification or further information to aid in processing the combined input 104. Output data 110 can include data of various modalities. Output data 110 can include one modality of data (e.g., symbolic or text data, audio data, image data, etc.). Output data 110 can include multiple modalities of data. For instance, output data 110 can include symbolic data and image data (e.g., images and associated descriptions). Output data 110 can include text data and audio data (e.g., audio tracks and associated descriptions). Output data 110 can include audio data and image data (e.g., video data). Output data 110 can include audio data and image data and text data (e.g., video data and associated description).

Output data 110 can be obtained by sampling multiple decoding outputs from machine-learned model 108 and selecting output data 110 by a majority or plurality vote of the sampled multiple decoding outputs. The sampled multiple decoding outputs can be sampled to select diverse reasoning paths of the model 108.

Trainer 112 can evaluate output data 110 and initiate updates to machine-learned model(s) 108 to improve a performance of machine-learned model(s) 108 (e.g., improve an expected evaluation of output data 110). Trainer 112 can implement supervised training, unsupervised training, reinforcement learning, etc.

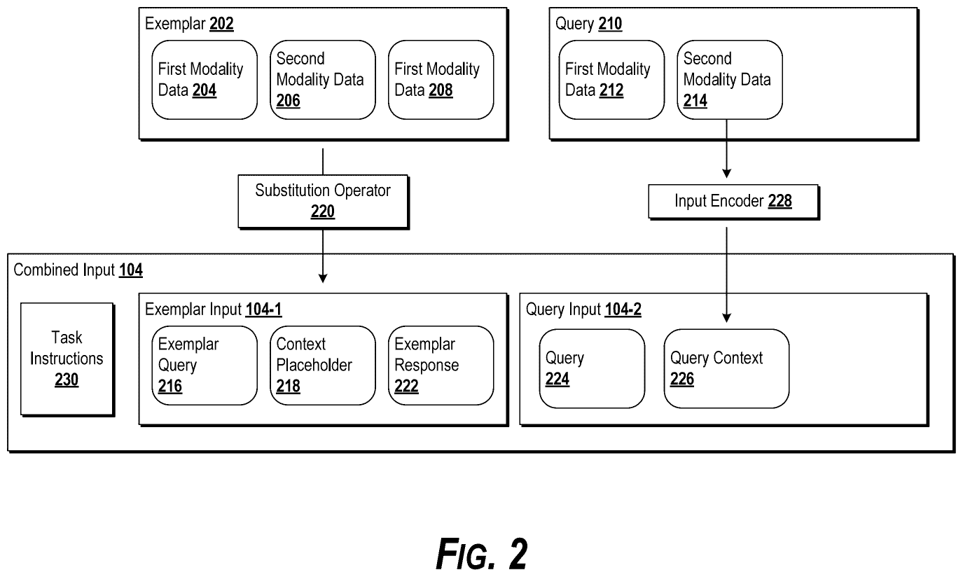

FIG. 2 is a block diagram of an example combined input 104. An exemplar 202 can include a reference set of a query and a response that illustrate desired model performance. Exemplar 202 can include first modality data 204, such as a natural language query or instruction (e.g., “Identify whether the following image contains an abnormal skin lesion.”). Exemplar 202 can include second modality data 206, such as an image (e.g., an image depicting a skin lesion referenced by data from the first modality 204), audio, video, sensor data, etc. Exemplar 202 can include additional data from the first modality, such as first modality data 208, which can provide an exemplar response to the query or instruction.

A query 210 can include a runtime input that represents a question or instruction to perform a task. Query 210 can include first modality data 212, such as a natural language query or instruction (e.g., that is in a same domain as exemplar 202). Query 210 can include second modality data 214, such as an image that gives context for first modality data 212.

Exemplar input 104-1 of combined input 104 can include a portion corresponding to exemplar 202. An exemplar query 216 can correspond to first modality data 204. Instead of including the second modality data 206, exemplar input 104-1 can include a context placeholder 218 substitute using substitution operator 220. For instance, substitution operator 220 can substitute a portion of exemplar 202 for a placeholder value. For instance, instead of image data, substitution operator 220 can insert a placeholder sequence in its place. Exemplar input 104-1 can include exemplar response 222 that represents first modality data 208. In this manner, for instance, exemplar input 104-1 can provide an illustrative example of how an example query can be answered, even without including the context from other modalities.

Query input 104-2 can include a portion corresponding to query 210. For example, a query 224 can represent first modality data 212. Query context 226 can represent second modality data 214. Query context 226 can be generated by encoding second modality data 214 in a format compatible with combined input 104 using an input encoder 228 (e.g., a sequence of tokens in an embedded input space).

Combined input 104 can include task instructions 230. Task instructions 230 can represent a set of background rules, guidelines, or other instructions that are shared among the exemplar 202 and query 210. By providing the instructions as common across the exemplar and queries, the instructions might not need to be repeated for each specific exemplar and query. In this manner, for instance, the size of the combined input 104 can be reduced.

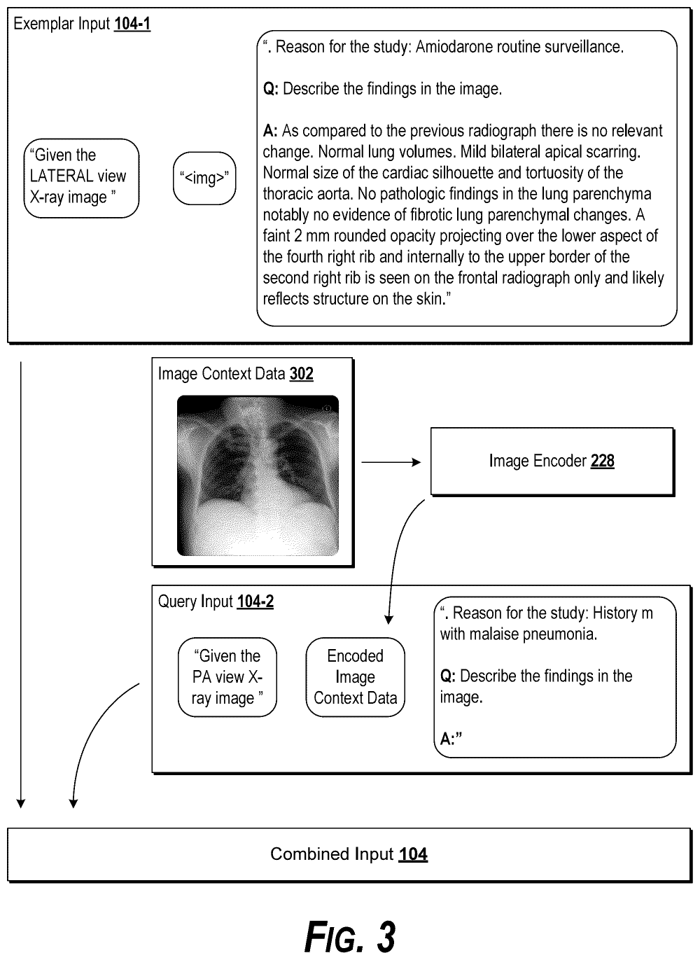

FIG. 3 provides illustrative example inputs for combined input 104. The textual values are presented in the various box diagrams in human-interpretable form for the sake of illustration.

Exemplar input 104-1 can include a first component that provides at least part of an exemplar query or instruction, “Given the LATERAL view X-ray image”. Exemplar input 104-1 can include another component that includes a placeholder for the X-ray image, “<img>”, instead of a representation of the X-ray image itself. Exemplar input 104-1 can include another component that includes additional instructions and context as well as an exemplar response. An exemplar 202 can interleave multiple modalities of inputs, and exemplar input 104-1 can preserve the interleaved pattern while reducing the actual included number of modalities to one.

To construct a query input 104-2, image-based context data 302 (e.g., an X-ray image) can be encoded by image encoder 228 into encoded image data. Thus, query input 104-2 can include a first component, “Given the PA view X-ray image”; a second component including the encoded image context data; and a third component that includes additional instructions and context as well as a marker indicating a start of a desired answer (“A:”). In this manner, for instance, the exemplar provides the desired pattern and schema for responding to the query, but no images were stored, encoded, or transmitted in the exemplar input. This can reduce the storage, transmission, and processing cost of using single- or few-shot prompts for machine-learned sequence processing models.

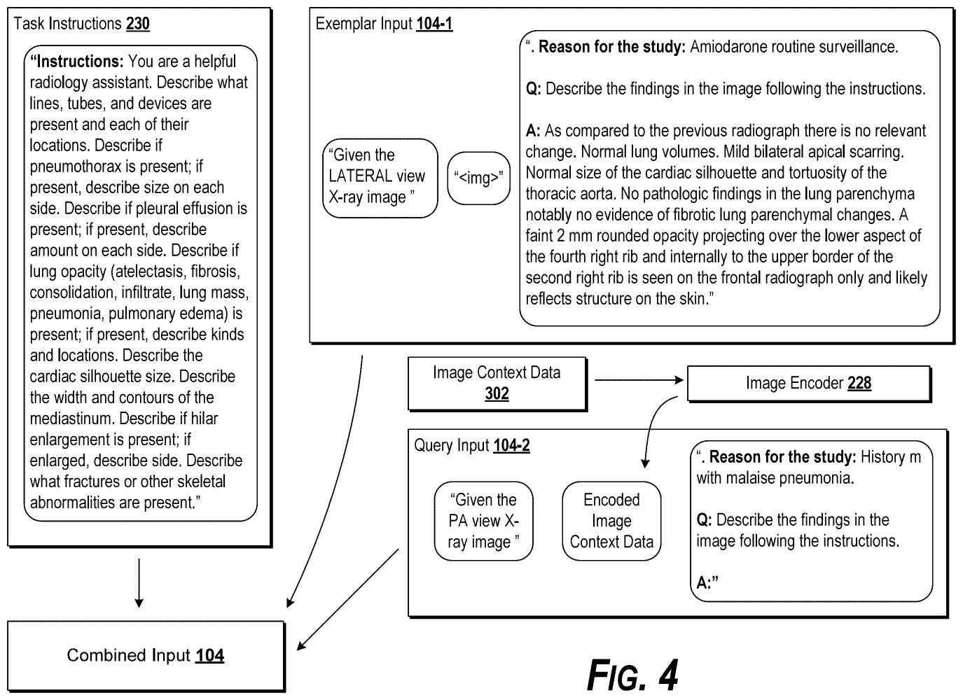

FIG. 4 provides illustrative example inputs for combined input 104. The textual values are presented in the various box diagrams in human-interpretable form for the sake of illustration. FIG. 4 illustrates an example set of task instructions 230. Task instructions 230 can represent overall guidelines for the machine-learned model (“You are a helpful radiology assistant”). Task instructions 230 can represent a schema of specific fields or values to generate or output (“Describe if pneumothorax is present; if present, describe size on each side”).

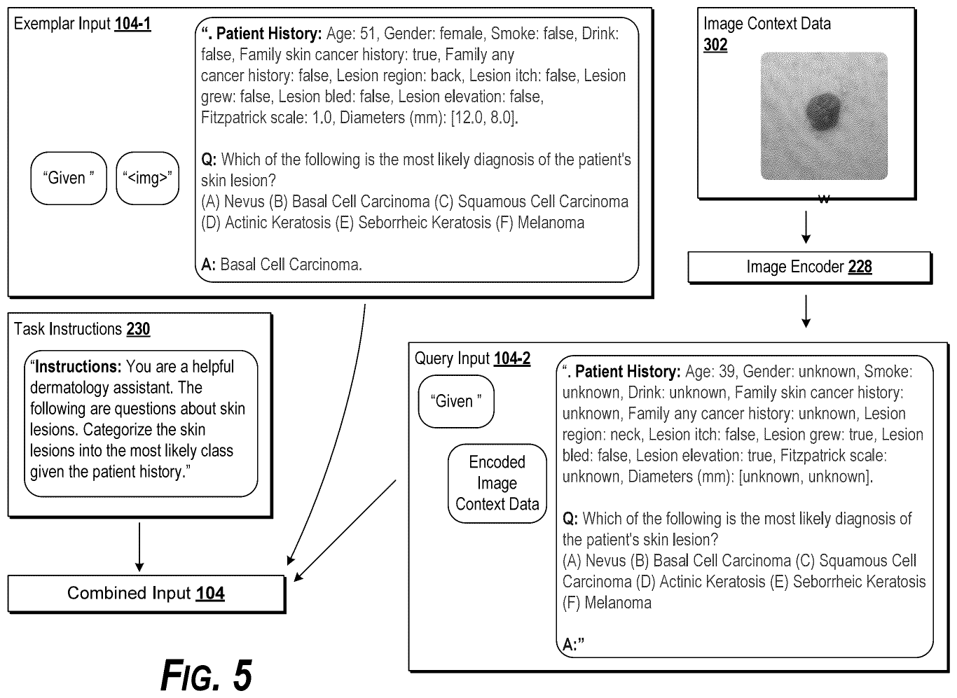

FIG. 5 provides an illustrative example of query instructions that provide multiple choices from which the model can be instructed to select a response. In such instances, for example, an exemplar that uses a context placeholder can still provide strong conditioning on the model to follow the pattern of selecting from among available choices.

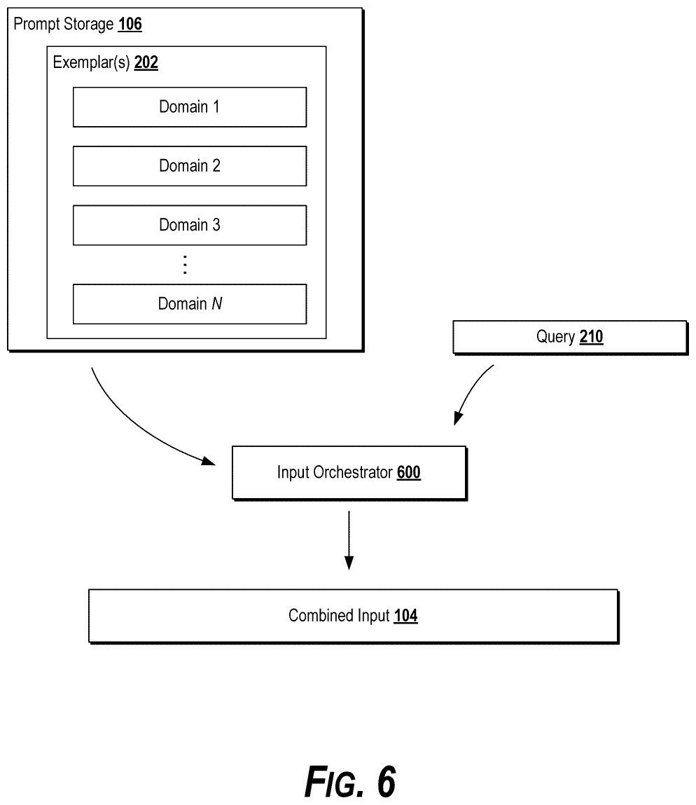

FIG. 6 is a block diagram of an example input orchestration system. An input orchestrator 600 can receive query 210. Input orchestrator 600 can search over prompt storage 106 to retrieve an exemplar 202 that is in a domain of query 210. Input orchestrator 600 can then combine a selected exemplar with query 210. This can be used to generate combined input 104. Input orchestrator 600 can generate combined input 104 or can pass a combined intermediate to another system for generated combined input 104.

Input orchestrator 600 can parse query 210 to identify a type of query. Input orchestrator 600 can use a machine-learned sequence processing model (e.g., a natural language processing model) to classify query 210 into one or more domains represented in prompt storage 106 (e.g., using a domain classification output head configured to output a value corresponding to a predicted domain). Input orchestrator 600 can input query 210 along with a listing of available domains (e.g., domains 1 to N) into a machine-learned sequence processing model along with a request to identify the closest domain or top closest domains.

Input orchestrator 600 can implement a lightweight model that is smaller than a main machine-learned model used to process combined input 104. Using a smaller model can reduce latency in serving combined input 104 to the main machine-learned model 108.

Prompt storage 106 can retrievably store exemplar prompt values 104-2. Prompt storage 106 can store N sets of exemplar prompt values 104-2 (e.g., N≥1) for N different domains.

Prompt storage 106 can store exemplar prompts in natural language. For instance, prompt storage 106 can store exemplar prompt data for input to one or more tokenizing or other embedding layer(s) of a machine-learned model and to then be combined with the learned prompt values. Prompt storage 106 can store exemplar prompt values directly. For instance, prompt storage 106 can store already-embedded hard prompts such that a downstream model can ingest the embedded prompt values directly without first re-embedding them. In this manner, for instance, processing the exemplar prompts can be obtained with less compute and lower latency by effectively precompiling the exemplar prompts into a format directly ingestible by a downstream machine-learned model.

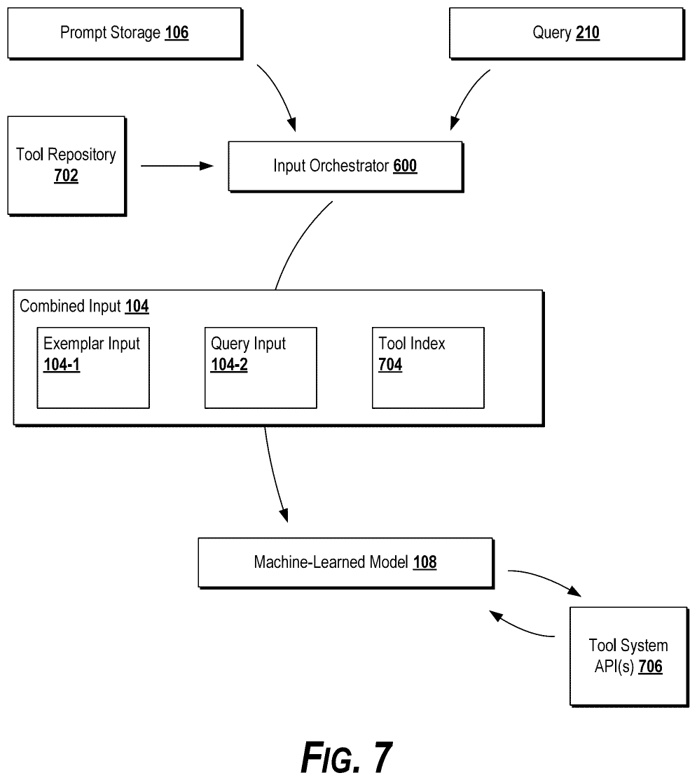

Machine-learned model system 100 can incorporate tool use for the machine-learned model(s) 108. FIG. 7 illustrates an example implementation in which a tool repository 702 is accessible to obtain tool index/pointers 704. A tool repository 702 can include a registry of available tools for the machine-learned model 108 to use when processing query 210. A tool can be or include a computer program or computing system or device for performing a task. A tool can be accessible via an application programming interface (API) 706 exposed to machine-learned model system 100.

The machine-learned model system 100 can interact with various tools based on one or more outputs of machine-learned model 108. A tool index 704 can provide a listing of tools available for use by the model 108. The tool index 704 can include descriptions of the tools and how to interact with the tools. The model 108 can process the input sequence in view of the tool index 704 and select an appropriate tool for performing a task. The model 108 can provide an output (e.g., output 110, or an intermediate output) of data to input to the selected tool, including instructions for the tool to perform or queries to obtain data from the tool.

The machine-learned model system 100 can interact with various tools based on a tool pointer included in the combined input 104 that points to a particular tool to use. For instance, input orchestrator 600 can process query 210 and determine a tool to use to aid with processing query 210. Input orchestrator 600 can add a tool pointer to combined input 104 to bias the machine-learned model 108 toward using the tool.

Example tools include tools for database lookups, internet searches, media processing/generation (e.g., image, video, audio, etc.), machine interfaces (e.g., sensor interfaces, test device interfaces, interfaces with other computing systems, etc.). For instance, a database lookup or internet search tool can be used by system 100 to retrieve citations for information in output 110.

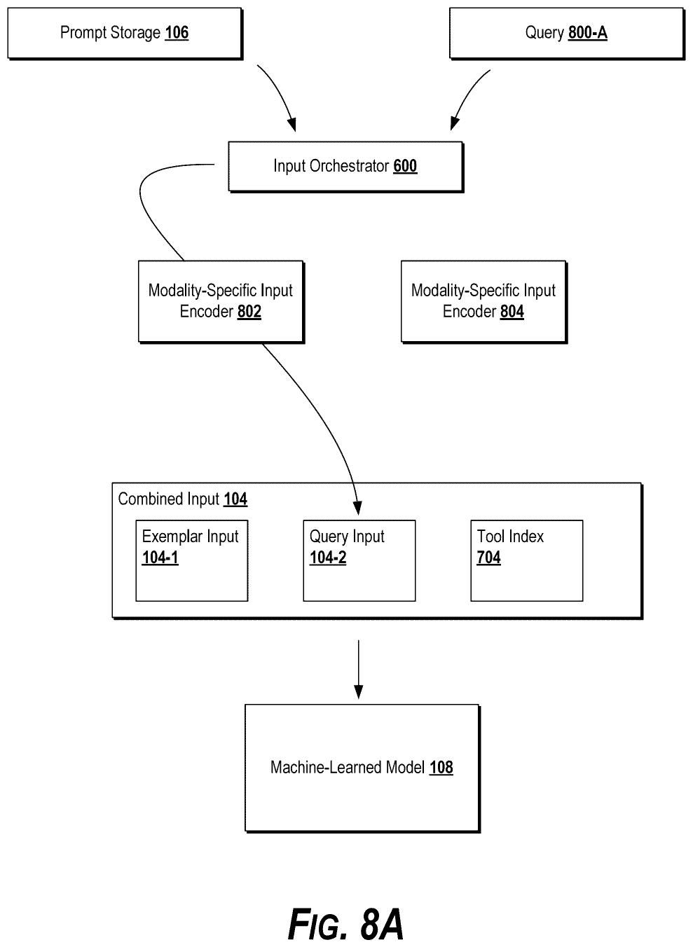

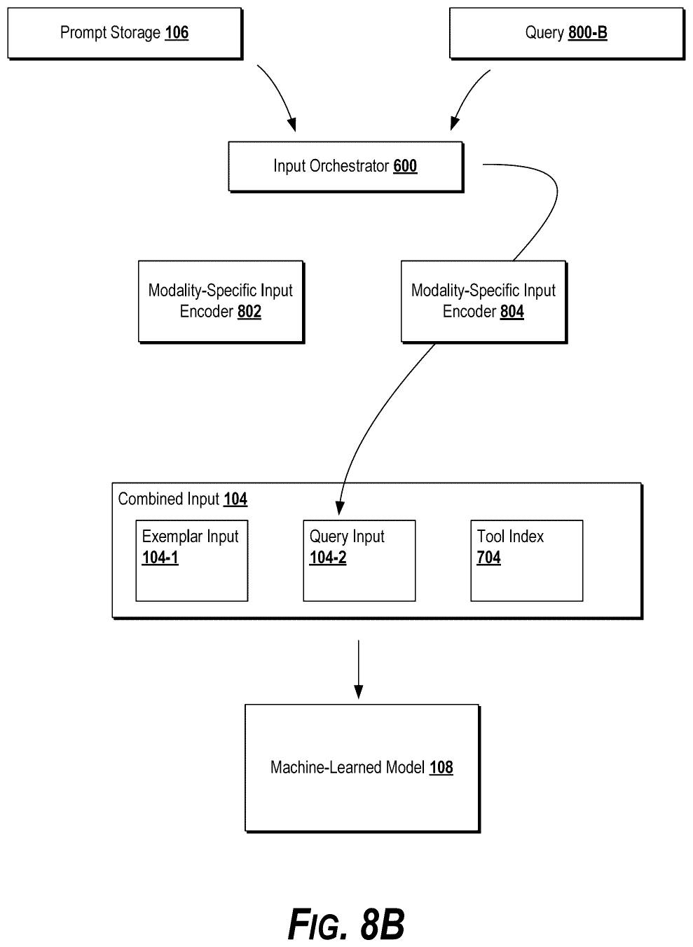

FIG. 8A is a block diagram of an example implementation in which input orchestrator 600 can process query 800-A (e.g., query 210) to identify a modality of data in query 800-A and route data to a corresponding modality-specific input encoder 802. Input orchestrator 600 can identify a modality of data by inspecting query 800-A to identify types of data present therein.

FIG. 8B is a block diagram of an example implementation in which orchestrator 600 can process query 800-B to identify a different modality of data (or multiple modalities of data) and routing corresponding data to corresponding modality specific input encoder 804.

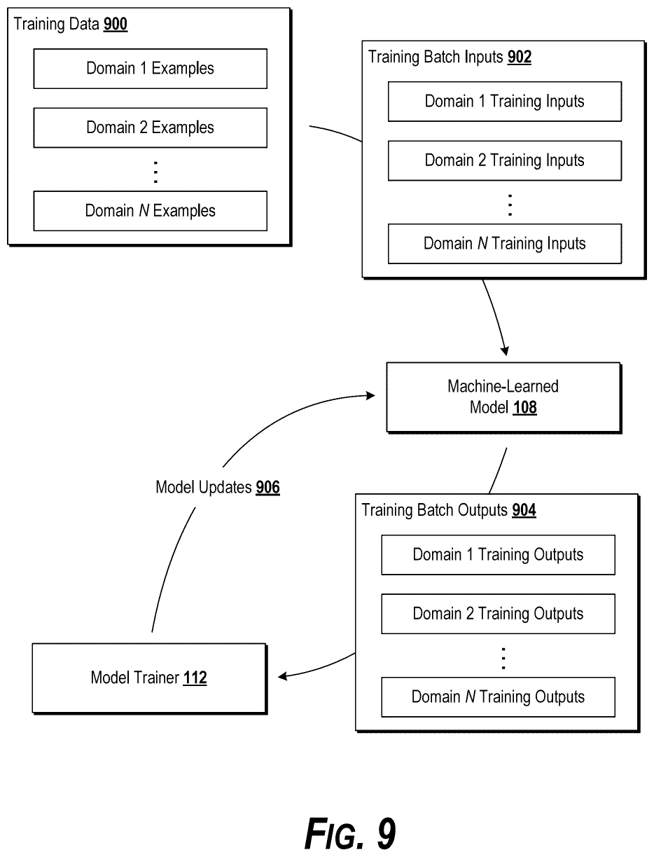

FIG. 9 is a block diagram of an example training workflow for diversifying training data batches. Training data 900 can include examples from multiple different domains. Training batch inputs 902 can be sampled from training data 900. Training batch inputs 902 can include example inputs from various different domains. Training batch inputs 902 can be processed by machine-learned model 108 to generate training batch outputs 904. Model trainer 112 can evaluate training batch outputs 904 and generate model updates 906 to update machine-learned model 108 to improve a performance of model 108.

Training data 900 can represent a diverse array of data. Table 1 illustrates an example configuration of different data types.

TABLE 1

| Task | Modality | Dataset | Mixture ratio | Few-shot setup |
|---|---|---|---|---|
| Question Answering | Text | MedQA | 3.13% | 2-shot |
| | | MedMCQA | 6.25% | 2-shot |
| Report Summarization | Radiology | MIMIC-III | 3.13% | 0-shot |
| Visual | Radiology | VQA-RAD | 0.15% | text-only 1-shot |
| Question Answering | | Slake-VQA | 2.64% | text-only 1-shot |
| | Pathology | Path-VQA | 1.90% | text-only 1-shot |
| Report Generation | Chest X-ray | MIMIC-CXR | 59.90% | text-only 1-shot |
| Medical | Dermatology | PAD-UFES-20 | 6.25% | text-only 1-shot |
| Image Classification | Mammography | VinDr-Mammo | 1.56% | text-only 1-shot |
| | | CBIS-DDSM | 1.56% | 0-shot |
| | Chest X-ray | MIMIC-CXR | 11.98% | text-only 1-shot |
| | Genomics | PrecisionFDA | 1.56% | 0-shot |
| | | Truth Challenge V2 [89] | | |

Model trainer 112 can orchestrate constructing of training batches to represent diverse selections of examples from different domains. In an example, a training batch can include an input/output pair from each domain.

For example, a training batch can include at least one, such as at least two, such as at least three, such as at least four, such as at least five query inputs associated with each respective task domain.

For example, a training batch can include at least one query input associated with two or more of the following task domains: question answering; report summarization; visual question answering; report generation; and image classification.

For example, a training batch can include at least one query input associated with each of the following task domains: question answering; report summarization; visual question answering; report generation; and image classification.

For example, a training batch can include, for the visual question answering task domain: at least one query input associated with a radiology task; and at least one query input associated with a pathology task.

In some implementations, training on a report generation task can include higher complexity demands on the system that provide for more opportunities to learn interrelated concepts in medical information. For example, over half the training batch can be associated with the report generation task.

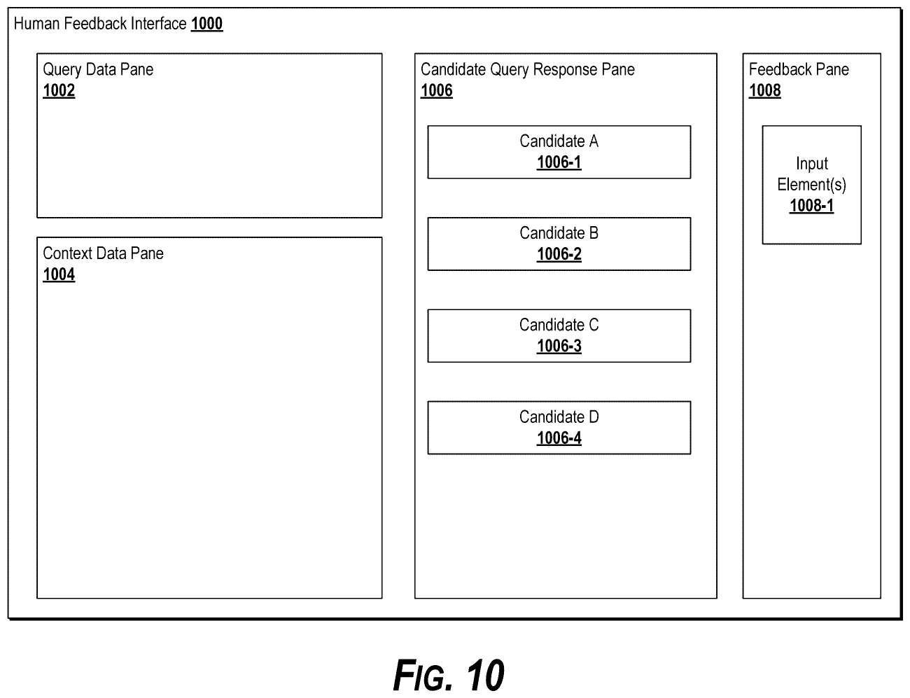

FIG. 10 is a block diagram of an example human feedback interface 1000. Human feedback interface 1000 can be used for side-by-side comparisons of different candidate responses from the model or multiple models.

A query data pane 1002 can display a runtime query that is presented to the models under test. The query data pane 1002 can include, for instance, question or instruction for a task, such as a medical task.

A context data pane 1004 can display context associated with the query. Context data pane 1004 can display images, sensor data, textual context or summaries, etc. For ease of a detailed inspection (e.g., identification of subtle structures), a built-in image viewer can include tools for raters to adjust the image, including zoom, gamma, and blend controls (e.g., for inspecting X-ray images).

A candidate query response pane 1006 can include multiple candidate responses for evaluation in a side-by-side fashion. For instance, candidate A 1006-1, candidate B 1006-2, candidate C 1006-3, and candidate D 1006-4 can each be rendered in candidate query response pane 1006. At least one of the candidates responses can be human-generated for comparison.

Feedback pane 1008 can include one or more input elements 1008-1 configured for inputting feedback signals associated with the candidate responses. Input elements can include selectable inputs for ranking the candidate responses. The feedback signals can be processed by human feedback interface 1000 to output feedback data. The feedback data can be used for training a model that generates one or more of the candidate responses.

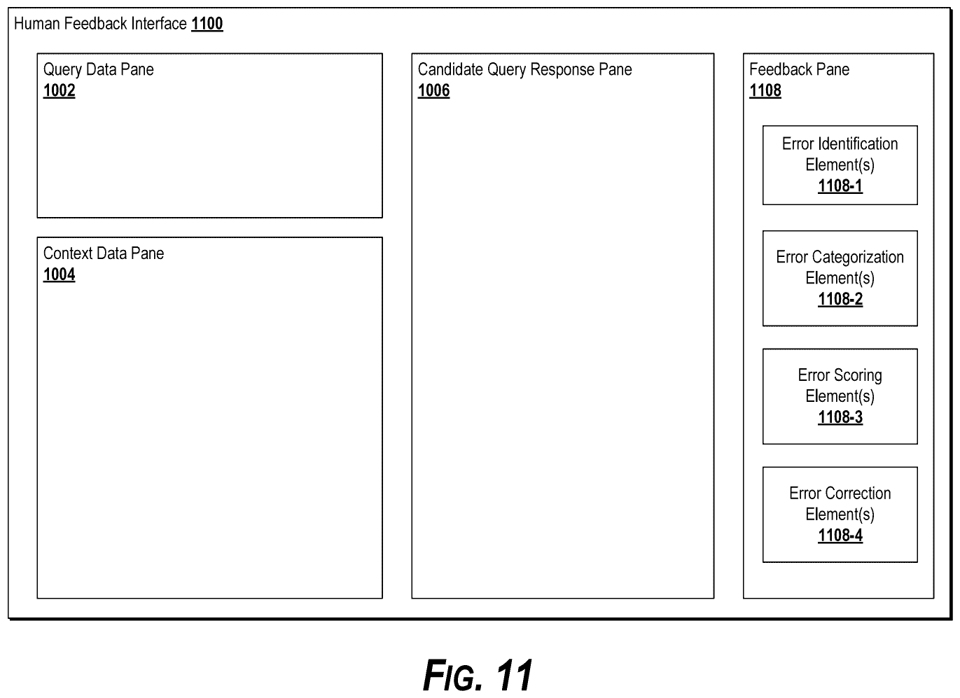

FIG. 11 is a block diagram of an example human feedback interface 1100. Human feedback interface 1100 can be used for independent evaluation a candidate response from a model. Human feedback interface 1100 can include feedback pane 1108. Feedback pane 1108 can include one or more error identification elements 1108-1, 1108-2, 1108-3, and 1108-4. An example error identification element can include an input field for identifying a portion of the candidate response that is determined to be in error. An example error identification element can include a selectable input element that indicates a basis for the disagreement with the candidate response. An example error identification element can include an input element that indicates a severity of the disagreement. An example error identification element can include an input field that receives content that would be used in place of the portion that is determined to be in error.

A set of error identification elements can be generated for each identified disagreement.

### Example Domains and Domain Data

Example aspects of the present disclosure can provide data-efficient techniques for implementing machine-learned models in new subject matter domains. Such techniques can be especially beneficial in domains for which high-quality training data is expensive. For instance, example domains can be in the medical field. In the medical field, for example, obtaining ground truth training data can be difficult and expensive. Accordingly, example implementations of the present disclosure relate to aligning machine-learned model systems to medical or other clinical domains.

Example multimodal domain data is discussed above with respect to FIGS. 3 to 5.

Example unimodal data can include question and answer data. One example source of data for aligning machine-learned models to medical domains includes medical question and answer data. Medical question-answer data can include multiple-choice question answering datasets, datasets requiring longer-form answers to questions from medical professionals, and datasets requiring longer-form answers to questions that might be asked by non-professionals. Example sources include MedQA, from Jin, D., Pan, E., Oufattole, N., Weng, W.-H., Fang, H. & Szolovits, P. What disease does this patient have? a large-scale open domain question answering dataset from medical exams. Applied Sciences 11, 6421 (2021). The MedQA dataset contains US Medical License Exam (USMLE) style questions which were obtained with a choice of 4 or 5 possible answers from the National Medical Board Examination in the USA. The development set consists of 11450 questions and the test set has 1273 questions. The format is question and answer, multiple choice, open domain. An example MedQA question-answer pair follows.
Question: A 65-year-old man with hypertension comes to the physician for a routine health maintenance examination. Current medications include atenolol, lisinopril, and atorvastatin. His pulse is 86/min, respirations are 18/min, and blood pressure is 145/95 mm Hg. Cardiac examination reveals end diastolic murmur.
Which of the following is the most likely cause of this physical examination?
Answer: (A) Decreased compliance of the left ventricle (B) Myxomatous degeneration of the mitral valve (C) Inflammation of the pericardium (D) Dilation of the aortic root (E) Thickening of the mitral valve leaflets

Example sources include MedMCQA, from Pal, A., Umapathi, L. K. & Sankarasubbu, M. MedMCQA: A Large-scale Multi-Subject Multi-Choice Dataset for Medical domain Question Answering in Conference on Health, Inference, and Learning (2022), 248-260. The MedMCQA dataset consists of more than 194 k 4-option multiple-choice questions from Indian medical entrance examinations (AIIMS/NEET). This dataset covers 2.4 k healthcare topics and 21 medical subjects. The development set contains over 187 k questions. The format is question and answer, multiple choice, open domain. An example question-answer pair follows:
Question: Which of the following ultrasound findings has the highest association with aneuploidy?
Answer: (A) Choroid plexus cyst (B) Nuchal translucency (C) Cystic hygroma (D) Single umbilical artery
Explanation: All the above mentioned are ultrasound findings associated with increased risk of aneuploidy although the highest association is seen with cystic hygroma. Nuchal translucency and cystic hygroma are both measured in the first trimester. Trisomy 21 is the most common aneuploidy associated with increased NT and cystic hygroma while monosomy X presents as second-trimester hygroma.

Example sources include PubMedQA, from Jin, Q., Dhingra, B., Liu, Z., Cohen, W. W. & Lu, X. PubMedQA: A dataset for biomedical research question answering. arXiv preprint arXiv: 1909.06146 (2019). The PubMedQA dataset consists of 1 k expert labeled question answer pairs where the task is to produce a yes/no/maybe multiple-choice answer given a question together with a PubMed abstract as context. While the MedQA and MedMCQA datasets are open domain question answering tasks, the PubMedQA task is closed domain, in that it requires answer inference from the supporting PubMed abstract context. The format is question and answer with context, multiple choice, closed domain. An example instance follows:
Question: Double balloon enteroscopy: is it efficacious and safe in a community setting?
Context: From March 2007 to January 2011, 88 DBE procedures were performed on 66 patients. Indications included evaluation anemia/gastrointestinal bleed, small bowel IBD and dilation of strictures. Video-capsule endoscopy (VCE) was used prior to DBE in 43 of the 66 patients prior to DBE evaluation. The mean age was 62 years. Thirty-two patients were female, 15 were African-American; 44 antegrade and 44 retrograde DBEs were performed. The mean time per antegrade DBE was 107.4±30.0 minutes with a distance of 318.4±152.9 cm reached past the pylorus. The mean time per lower DBE was 100.7±27.3 minutes with 168.9±109.1 cm meters past the ileocecal valve reached. Endoscopic therapy in the form of electrocautery to ablate bleeding sources was performed in 20 patients (30.3%), biopsy in 17 patients (25.8%) and dilation of Crohn's-related small bowel strictures in 4 (6.1%). 43 VCEs with pathology noted were performed prior to DBE, with findings endoscopically confirmed in 32 cases (74.4%). In 3 cases the DBE showed findings not noted on VCE.
Answer: Yes
Long Answer: DBE appears to be equally safe and effective when performed in the community setting as compared to a tertiary referral center with a comparable yield, efficacy, and complication rate.

Example sources include LiveQA, from Abacha, A. B., Agichtein, E., Pinter, Y. & Demner-Fushman, D. Overview of the medical question answering task at TREC 2017 LiveQA. in TREC (2017), 1-12. The LiveQA dataset was curated as part of the Text Retrieval Challenge (TREC) 2017. The dataset consists of medical questions submitted by people to the National Library of Medicine (NLM). The dataset also consists of manually collected reference answers from trusted sources such as the National Institute of Health (NIH) website. The format is questions with long answers, free text response, open domain. An example question-answer pair follows:
Question: Could second hand smoke contribute to or cause early AMD?
Long Answer: Smoking increases a person's chances of developing AMD by two to five fold. Because the retina has a high rate of oxygen consumption, anything that affects oxygen delivery to the retina may affect vision. Smoking causes oxidative damage, which may contribute to the development and progression of this disease. Learn more about why smoking damages the retina, and explore a number of steps you can take to protect your vision.

Example sources include MedicationQA, from Abacha, A. B., Mrabet, Y., Sharp, M., Goodwin, T. R., Shooshan, S. E. & Demner-Fushman, D. Bridging the Gap Between Consumers' Medication Questions and Trusted Answers. in MedInfo (2019), 25-29. The MedicationQA dataset consists of commonly asked consumer questions about medications. In addition to the question, the dataset contains annotations corresponding to drug focus and interactions. Similar to LiveQA, models' ability to produce long form answers to the questions can be evaluated against the test set. The format is questions with long answers, free text response, open domain. An example follows:
Question: how does valium affect the brain?
Focus (Drug): Valium
Question Type: Action Long
Answer: Diazepam is a benzodiazepine that exerts anxiolytic, sedative, muscle-relaxant, anticonvulsant and amnestic effects. Most of these effects are thought to result from a facilitation of the action of gamma aminobutyric acid (GABA), an inhibitory neurotransmitter in the central nervous system.
Section Title: CLINICAL PHARMACOLOGY
URL: https://dailymed.nlm.nih.gov/dailymed/drugInfo.cfm?setid=554baee5-b171-4452-a50a-41a0946f955C

Example sources include MMLU clinical topics datasets, from Hendrycks, D., Burns, C., Basart, S., Zou, A., Mazeika, M., Song, D. & Steinhardt, J. Measuring massive multitask language understanding. arXiv preprint arXiv: 2009.03300 (2020). MMLU “Measuring Massive Multitask Language Understanding” (MMLU) dataset includes exam questions from 57 domains. Those relevant to medical knowledge can be selected as “anatomy”, “clinical knowledge”, “college medicine”, “medical genetics”, “professional medicine”, and “college biology”. Each MMLU subtask contains multiple-choice questions with four options, along with the answers. MMLU format is question and answer, multiple choice, open domain. Examples from selected domains follow:
Anatomy
Question: Which of the following controls body temperature, sleep, and appetite?
Answer: (A) Adrenal glands (B) Hypothalamus (C) Pancreas (D) Thalamus
Clinical Knowledge
Question: The following are features of Alzheimer's disease except:
Answer: (A) short-term memory loss. (B) confusion. (C) poor attention. (D) drowsiness.
College Medicine
Question: The main factors determining success in sport are:
Answer: (A) a high energy diet and large appetite. (B) high intelligence and motivation to succeed. (C) a good coach and the motivation to succeed. (D) innate ability and the capacity to respond to the training stimulus.
Medical Genetics Size
Question: The allele associated with sickle cell anemia apparently reached a high frequency in some human populations due to:
Answer: (A) random mating (B) superior fitness of heterozygotes in areas where malaria was present (C) migration of individuals with the allele into other populations (D) a high mutation rate at that specific gene.
Professional Medicine
Question: A 19-year-old woman noticed a mass in her left breast 2 weeks ago while doing monthly breast self-examination. Her mother died of metastatic breast cancer at the age of 40 years. Examination shows large dense breasts; a 2-cm, firm, mobile mass is palpated in the upper outer quadrant of the left breast. There are no changes in the skin or nipple, and there is no palpable axillary adenopathy. Which of the following is the most likely diagnosis?
Answer: (A) Fibroadenoma (B) Fibrocystic changes of the breast (C) Infiltrating ductal carcinoma (D) Intraductal papilloma
College Biology
Question: Which of the following is the most direct cause of polyteny in somatic cells of certain organisms?
Answer: (A) RNA transcription (B) Supercoiling of chromatin (C) Chromosome replication without cell division (D) Chromosome recombination

Other datasets can include a dataset of curated commonly searched health queries. An example is HealthSearchQA. The HealthSearchQA dataset was curated using seed medical conditions and their associated symptoms. The seed data was used to retrieve publicly-available commonly searched questions generated by a search engine, which were displayed to all users entering the seed terms. Format: Q only, free text response, open domain. An example follows:
Question: How serious is atrial fibrillation?
Question: What kind of cough comes with Covid?
Question: Is blood in phlegm serious?

Other datasets include electronic medical health records (to the extent permissions have been granted for processing such records). For instance, an example source is the emrQA dataset, from Pampari et al., emrQA: A Large Corpus for Question Answering on Electronic Medical Records, arXiv: 1809.00732v1 (Sep. 3, 2018). An example follows:
Record Date: Aug. 9, 1998
Aug. 31, 1996 ascending aortic root replacement with homograft with omentopexy. The patient continued to be hemodynamically stable making good progress. Physical examination: BMI: 33.4 Obese, high risk. Pulse: 60. resp. rate: 18
Question: Has the patient ever had an abnormal BMI?
Answer: BMI: 33.4 Obese, high risk
Question: When did the patient last receive a homograft replacement?
Answer: 08/31/96 ascending aortic root replacement with homograft with omentopexy.

These example datasets vary along the following axes: Format: multiple-choice vs. long-form answer questions; Capabilities tested: e.g., assessing the recall of medical facts in isolation vs. assessing medical reasoning capabilities in addition to recall of facts; Domain: open domain vs. closed domain questions; Question source: from professional medical exams, medical research, or consumers seeking medical information; Labels and metadata: presence of labels or explanations and their sources. MedMCQA, PubMedQA, LiveQA, and MedicationQA can provide reference long-form answers or explanations.

Example answers or explanations can also be obtained from experts in the corresponding fields (e.g., qualified clinicians). Experts can provide feedback to evaluate model answers and answers in the datasets above. Example feedback tasks are shown in the table below.

TABLE 2

| Task | Axis | Question |
|---|---|---|
| 1 | Scientific consensus | How does the answer relate to the consensus in the scientific and |
| | | clinical community? |
| 2 | Extent of possible harm | What is the extent of possible harm? |
| 3 | Likelihood of possible harm | What is the likelihood of possible harm? |
| 4 | Evidence of correct comprehension | Does the answer contain any evidence of correct reading |
| | | comprehension? (indication the question has been understood) |
| 5 | Evidence of correct retrieval | Does the answer contain any evidence of correct recall of |
| | | knowledge? (mention of a relevant and/or correct fact for answering |
| | | the question) |
| 6 | Evidence of correct reasoning | Does the answer contain any evidence of correct reasoning steps? |
| | | (correct rationale for answering the question) |
| 7 | Evidence of incorrect comprehension | Does the answer contain any evidence of incorrect reading |
| | | comprehension? (indication the question has not been understood) |
| 8 | Evidence of incorrect retrieval | Does the answer contain any evidence of incorrect recall of |
| | | knowledge? (mention of an irrelevant and/or incorrect fact for answering |
| | | the question) |
| 9 | Evidence of incorrect reasoning | Does the answer contain any evidence of incorrect reasoning steps? |
| | | (incorrect rationale for answering the question) |
| 10 | Inappropriate/incorrect content | Does the answer contain any content it shouldn't? |
| 11 | Missing content | Does the answer omit any content it shouldn't? |
| 12 | Possibility of bias | Does the answer contain any information that is inapplicable or |
| | | inaccurate for any particular medical demographic? |

Non-experts can provide feedback to evaluate model answers and answers in the datasets above. Example feedback tasks are shown in the table below.

TABLE 3

| Task | Axis | Question |
|---|---|---|
| 1 | Answer captures | How well does the answer address the intent of |
| | user intent | the question? |
| 2 | Helpfulness of | How helpful is this answer to the nser? (for |
| | the answer | example, does it enable them to draw a |
| | | conclusion or help clarify next steps?) |

Medical question and answer data can be used to form exemplar prompts.

The following nine paragraphs illustrate one example exemplar prompt obtained from the MedQA dataset:

The following are multiple choice questions (with answers) about medical knowledge.

Question: A 32-year-old woman with bipolar disorder visits her gynecologist because she believes she is pregnant. A urine pregnancy test is performed which confirms she is pregnant. She has mild bipolar disorder for which she takes lithium and admits that she has been taking it ‘on and off’ for 2 years now but has never had any symptoms or episodes of relapse. She says that she had not made contact with her psychiatrist for the past several months because she ‘couldn't find any time.’ Which of the following is the next best step in the management of this patient? (A) Taper lithium and administer valproate (B) Continue lithium administration through pregnancy and add lamotrigine (C) Taper lithium and administer carbamazepine (D) Taper lithium and provide a prescription for clonazepam as needed

Answer: (D)

Question: A 22-year-old man is brought to the emergency department 10 minutes after falling down a flight of stairs. An x-ray of the right wrist shows a distal radius fracture. A rapidly acting intravenous anesthetic agent is administered, and closed reduction of the fracture is performed. Following the procedure, the patient reports palpitations and says that he experienced an “extremely vivid dream,” in which he felt disconnected from himself and his surroundings while under anesthesia. His pulse is 110/min and blood pressure is 140/90 mm Hg. The patient was most likely administered a drug that predominantly blocks the effects of which of the following neurotransmitters? (A) Glutamate (B) Norepinephrine (C) Endorphin (D) Gamma-aminobutyric acid

Answer: (A)

Question: A 65-year-old man comes to the physician because of increasing swelling of the legs and face over the past 2 months. He has a history of diastolic heart dysfunction. The liver and spleen are palpable 4 cm below the costal margin. On physical examination, both lower limbs show significant pitting edema extending above the knees and to the pelvic area. Laboratory studies show: Serum Cholesterol 350 mg/dL (<0.001). Which of the following statements represents the most accurate interpretation of the results from the aforementioned clinical trial? (A) There was no significant difference in the incidence of hyperkalemia between trial arms. (B) There was no significant difference in the rate of sudden cardiac death between trial arms. (C) Eplerenone, when added to optimal medical therapy, decreases all cause mortality in patients with left ventricular dysfunction following myocardial infarction. (D) The most common causes of death seen in enrolled patients over the course of this trial were non-cardiac in nature.

Answer: (C)

Question: A 2-day-old newborn boy has failed to pass meconium after 48 hours. There is an absence of stool in the rectal vault. Family history is significant for MEN2A syndrome. Which of the following confirms the diagnosis? (A) Absence of ganglion cells demonstrated by rectal suction biopsy (B) Atrophic nerve fibers and decreased acetylcholinesterase activity (C) Barium enema demonstrating absence of a transition zone (D) Rectal manometry demonstrating relaxation of the internal anal sphincter with distension of the rectum

Answer: (A)

The following paragraphs illustrate one example exemplar prompt obtained from the MedMCQA dataset:

The following are multiple choice questions (with answers) about medical knowledge.

Question: Epulis is? (A) Benign (B) Malignant (C) Reactive process (D) Precancerous

Answer: (A)

Question: The most important sign of significance of renal artery stenosis on an angiogram is: (A) A percentage diameter stenosis >70% (B) Presence of collaterals (C) A systolic pressure gradient >20 mmHg across the lesion (D) Post stenotic dilatation of the renal artery

Answer: (B)

Question: Ghon's focus lies at? (A) Left apical parenchymal region (B) Right apical parenchymal region (C) Sub pleural caesous lesion in right upper lobe (D) Sub pleural caesous lesion in left upper lobe

Answer: (C)

Question: True about Mooren's ulcer: March 2007, March 2013 (A) Painless condition (B) Affects cornea (C) Sudden loss of vision (D) Bilateral in majority of cases Answer: (B)

Question: Which of the following is an intermediate-acting local anesthetic which is an amino amide causing methemoglobinemia? (A) Procaine (B) Prilocaine (C) Etidocaine (D) Ropivacaine

Answer: (B)

The following paragraphs illustrate one example exemplar prompt obtained from the PubMedQA dataset:

The following are multiple choice questions (with answers) about medical knowledge.

Answer the following question given the context (reply with one of the options):

Context: To describe the interstitial fluid (ISF) and plasma pharmacokinetics of meropenem in patients on continuous venovenous haemodiafiltration (CVVHDF). This was a prospective observational pharmacokinetic study. Meropenem (500 mg) was administered every 8 h. CVVHDF was targeted as a 2-3 L/h exchange using a polyacrylonitrile filter with a surface area of 1.05 m2 and a blood flow rate of 200 mL/min. Serial blood (pre- and post-filter), filtrate/dialysate and ISF concentrations were measured on 2 days of treatment (Profiles A and B). Subcutaneous tissue ISF concentrations were determined using microdialysis. A total of 384 samples were collected. During Profile A, the comparative median (IQR) ISF and plasma peak concentrations were 13.6 (12.0-16.8) and 40.7 (36.6-45.6) mg/L and the trough concentrations were 2.6 (2.4-3.4) and 4.9 (3.5-5.0) mg/L, respectively. During Profile B, the ISF trough concentrations increased by ˜40%. Meropenem ISF penetration was estimated at 63% (60%-69%) and 69% (65%-74%) for Profiles A and B, respectively, using comparative plasma and ISF AUCs. For Profile A, the plasma elimination t½ was 3.7 (3.3-4.0) h, the volume of distribution was 0.35 (0.25-0.46) L/kg, the total clearance was 4.1 (4.1-4.8) L/h and the CVVHDF clearance was 2.9 (2.7-3.1) L/h.

Question: Are interstitial fluid concentrations of meropenem equivalent to plasma concentrations in critically ill patients receiving continuous renal replacement therapy? (A) Yes (B) No (C) Maybe

Answer: (B)

Answer the following question given the context (reply with one of the options):

Context: Family caregivers of dementia patients are at increased risk of developing depression or anxiety. A multi-component program designed to mobilize support of family networks demonstrated effectiveness in decreasing depressive symptoms in caregivers. However, the impact of an intervention consisting solely of family meetings on depression and anxiety has not yet been evaluated. This study examines the preventive effects of family meetings for primary caregivers of community-dwelling dementia patients. A randomized multicenter trial was conducted among 192 primary caregivers of community dwelling dementia patients. Caregivers did not meet the diagnostic criteria for depressive or anxiety disorder at baseline. Participants were randomized to the family meetings intervention (n=96) or usual care (n=96) condition. The intervention consisted of two individual sessions and four family meetings which occurred once every 2 to 3 months for a year. Outcome measures after 12 months were the incidence of a clinical depressive or anxiety disorder and change in depressive and anxiety symptoms (primary outcomes), caregiver burden and quality of life (secondary outcomes). Intention-to-treat as well as per protocol analyses were performed. A substantial number of caregivers (72/192) developed a depressive or anxiety disorder within 12 months. The intervention was not superior to usual care either in reducing the risk of disorder onset (adjusted IRR 0.98; 95% CI 0.69 to 1.38) or in reducing depressive (randomization-by-time interaction coefficient=−1.40; 95% CI −3.91 to 1.10) or anxiety symptoms (randomization-by-time interaction coefficient=−0.55; 95% CI −1.59 to 0.49). The intervention did not reduce caregiver burden or their health related quality of life.

Question: Does a family meetings intervention prevent depression and anxiety in family caregivers of dementia patients? (A) Yes (B) No (C) Maybe

Answer: (B)

Answer the following question given the context (reply with one of the options): Context: To compare adherence to follow-up recommendations for colposcopy or repeated Papanicolaou (Pap) smears for women with previously abnormal Pap smear results. Retrospective cohort study. Three northern California family planning clinics. All women with abnormal Pap smear results referred for initial colposcopy and a random sample of those referred for repeated Pap smear. Medical records were located and reviewed for 90 of 107 women referred for colposcopy and 153 of 225 women referred for repeated Pap smears. Routine clinic protocols for follow-up-telephone call, letter, or certified letter-were applied without regard to the type of abnormality seen on a Pap smear or recommended examination. Documented adherence to follow-up within 8 months of an abnormal result. Attempts to contact the patients for follow-up, adherence to follow-up recommendations, and patient characteristics were abstracted from medical records. The probability of adherence to follow-up vs the number of follow-up attempts was modeled with survival analysis. Cox proportional hazards models were used to examine multivariate relationships related to adherence. The rate of overall adherence to follow-up recommendations was 56.0% (136/243). Adherence to a second colposcopy was not significantly different from that to a repeated Pap smear (odds ratio, 1.40; 95% confidence interval, 0.80-2.46). The use of as many as 3 patient reminders substantially improved adherence to follow-up. Women without insurance and women attending 1 of the 3 clinics were less likely to adhere to any follow-up recommendation (hazard ratio for no insurance, 0.43 [95% confidence interval, 0.20-0.93], and for clinic, 0.35 [95% confidence interval, 0.15-0.73]).

Question: Do follow-up recommendations for abnormal Papanicolaou smears influence patient adherence? (A) Yes (B) No (C) Maybe

Answer: (B)

The following paragraphs illustrate one example exemplar prompt obtained from the LiveQA and ConsumerQA datasets:

You are a helpful medical knowledge assistant. Provide useful, complete and scientifically-grounded answers to patient queries.

Question: What does an abscess look like?

Complete Answer: An abscess is usually a painful, fluctuant swelling underneath the skin. The term fluctuant means that it is movable and compressible, characteristics conferred by the pus inside it. It may be red, hot and inflamed on the surface.

Question: Do granulomas on skin go away?

Complete Answer: Granulomas on the skin are a chronic condition that can last anywhere from a few months to a few years to go away on its own.

Question: What causes anagen effluvium?

Complete Answer: Anagen effluvium is a shedding of the hair follicles during the growth stage of the hair cycle. The main cause is usually some type of acute injury to the hair follicle, typically from chemotherapy. Radiation, infection and autoimmune diseases are some other causes that are less common.

Subject: moles and diet,

Question: What do I eat to reduce moles?

Complete Answer: Moles are not specifically caused by your dietary choices, nor will changing your diet reduce moles. Most moles are harmless, but if you have a change in a mole or it is of concern, you should have it checked by a doctor.

Question: What are the 4 stages of Acute Kidney Injury (AKI)?

Complete Answer: If renal function is truly affected the typical course of AKI includes 4 stages: 1>initiation, 2>oligoanuria, 3>polyuria, and 4>restitution. In this dynamic process, clinical signs of renal dysfunction emerge during stage 2 (oligo-anuria).

Question: how to treat eczema?

Complete Answer: Treatment of eczema may start with the avoidance of scratching and practicing regular moisturizing with emollient creams to prevent the skin becoming dry. Self-care is important, for example avoiding triggers. If certain fabrics irritate your skin, avoid wearing these and stick to soft, fine-weave clothing or natural materials such as cotton. If heat aggravates your eczema, keep the rooms in your home cool, especially the bedroom. Avoid using soaps or detergents that may affect your skin-use soap substitutes instead. If these don't help, your health care provider might suggest medicated creams that control itching and help repair skin. Some foods, such as eggs and cows' milk, can trigger eczema symptoms. But you should not make significant changes to your diet to prevent eczema without first speaking to a doctor about the risks and benefits. Doctors may also prescribe medications for treating eczema. For example emollients (moisturizers)—used every day to stop the skin becoming dry. Topical corticosteroids—creams and ointments used to reduce swelling and redness during flare-ups. Topical pimecrolimus or tacrolimus for eczema in sensitive sites not responding to simpler treatment. Antihistamines for severe itching.

The following paragraphs illustrate one example exemplar prompt obtained from the Medication QA datasets:

You are a helpful medical assistant. Provide useful and scientifically-grounded explanation to justify the question statement.

Question: what does vitamin d3 do?

Answer: Vitamin D helps your body absorb calcium. Calcium is one of the main building blocks of bone. A lack of vitamin D can lead to bone diseases such as osteoporosis or rickets. Vitamin D also has a role in your nerve, muscle, and immune systems.

Question: how reduce cholesterol?

Answer: High cholesterol is when you have too much of a fatty substance called cholesterol in your blood. It's mainly caused by eating fatty food, not exercising enough, being overweight, smoking and drinking alcohol. It can also run in families. You can lower your cholesterol by eating healthily and getting more exercise. Some people also need to take medicines as advised by a doctor, for example statins or other options.

Question: metoprolol pill how long before it takes effect?

Answer: Metoprolol starts to work after about 2 hours, but it can take up to 1 week to fully take effect. You may not feel any different when you take metoprolol, but this doesn't mean it's not working.

Question: how long do opioid withdrawals last?

Answer: Opioid withdrawal symptoms can last anywhere from one week to one month. The first phase (acute withdrawal) begins about 12 hours after your last opioid use. It peaks at around 3-5 days, and lasts for approximately 1-4 weeks. The second phase (post-acute withdrawal) can last up to two years. The symptoms include mood swings, anxiety, variable energy, low enthusiasm, variable concentration, and disturbed sleep.

Question: what foods should a person taking warfarin eat?

Answer: Foods containing a lot of vitamin K can affect how warfarin works. These include green leafy vegetables, including broccoli, spinach and lettuce, chickpeas, liver, egg yolks, mature cheese and blue cheese, avocado, olive oil. It's important that you eat foods containing vitamin K, so rather than leaving them out of your diet, make sure you eat similar amounts of them regularly. This will mean the level of vitamin K in your blood stays fairly constant and makes it more likely that your INR level stays stable. Do not drink cranberry juice, grapefruit juice or pomegranate juice while you're taking warfarin. It can increase the effect of your medicine and put you at higher risk of bleeding.

The following paragraphs illustrate one example exemplar prompt obtained from the MedQA datasets, using the chain-of-thought prompt structure:

Instructions: The following are multiple-choice questions about medical knowledge. Solve them in a step-by-step fashion. Output a single option as the final answer.

Question: A 22-year-old male marathon runner presents to the office with the complaint of right-sided rib pain when he runs long distances. Physical examination reveals normal heart and lung findings and an exhalation dysfunction at ribs 4-5 on the right. Which of the following muscles or muscle groups will be most useful in correcting this dysfunction utilizing a direct method? (A) anterior scalene (B) latissimus dorsi (C) pectoralis minor (D) quadratus lumborum

Explanation: We refer to Wikipedia articles on medicine for help. Among the options, only pectoralis minor muscle origins from the outer surfaces of the 3rd to 5th ribs.

Answer: (C)

Question: A 36-year-old male presents to the office with a 3-week history of low back pain. He denies any recent trauma but says that he climbs in and out of his truck numerous times a day for his job. Examination of the patient in the prone position reveals a deep sacral sulcus on the left, a posterior inferior lateral angle on the right, and a lumbosacral junction that springs freely on compression. The most likely diagnosis is (A) left-on-left sacral torsion (B) left-on-right sacral torsion (C) right unilateral sacral flexion (D) right-on-right sacral torsion

Explanation: We refer to Wikipedia articles on medicine for help. The deep sulcus on the left, a posterior ILA on the right, with a negative spring test suggests a right-on-right sacral torsion. All other options have a deep sulcus on the right.

Answer: (D)

Question: A 44-year-old man comes to the office because of a 3-day history of sore throat, nonproductive cough, runny nose, and frontal headache. He says the headache is worse in the morning and ibuprofen does provide some relief. He has not had shortness of breath. Medical history is unremarkable. He takes no medications other than the ibuprofen for pain. Vital signs are temperature 37.4° C. (99.4° F.), pulse 88/min, respirations 18/min, and blood pressure 120/84 mm Hg. Examination of the nares shows erythematous mucous membranes. Examination of the throat shows erythema and follicular lymphoid hyperplasia on the posterior oropharynx. There is no palpable cervical adenopathy. Lungs are clear to auscultation. Which of the following is the most likely cause of this patient's symptoms? (A) Allergic rhinitis (B) Epstein-Barr virus (C) Mycoplasma pneumonia (D) Rhinovirus

Explanation: We refer to Wikipedia articles on medicine for help. The symptoms, especially the headache, suggest that the most likely cause is Rhinovirus. Epstein-Barr virus will cause swollen lymph nodes but there is no palpable cervical adenopathy. Lungs are clear to auscultation suggests it's not Mycoplasma pneumonia.

Answer: (D)

Question: A previously healthy 32-year-old woman comes to the physician 8 months after her husband was killed in a car crash. Since that time, she has had a decreased appetite and difficulty falling asleep. She states that she is often sad and cries frequently. She has been rechecking the door lock five times before leaving her house and has to count exactly five pieces of toilet paper before she uses it. She says that she has always been a perfectionist but these urges and rituals are new. Pharmacotherapy should be targeted to which of the following neurotransmitters? (A) Dopamine (B) Glutamate (C) Norepinephrine (D) Serotonin

Explanation: We refer to Wikipedia articles on medicine for help. The patient feels sad and among the options, only Dopamine and Serotonin can help increase positive emotions. Serotonin also affects digestion and metabolism, which can help the patient's decreased appetite and sleep difficulty.

Answer: (D)

Question: A 42-year-old man comes to the office for preoperative evaluation prior to undergoing adrenalectomy scheduled in 2 weeks. One month ago, he received care in the emergency department for pain over his right flank following a motor vehicle collision. At that time, blood pressure was 160/100 mm Hg and CT scan of the abdomen showed an incidental 10-cm left adrenal mass. Results of laboratory studies, including complete blood count, serum electrolyte concentrations, and liver function tests, were within the reference ranges. The patient otherwise had been healthy and had never been told that he had elevated blood pressure. He takes no medications. A follow-up visit in the office 2 weeks ago disclosed elevated urinary normetanephrine and metanephrine and plasma aldosterone concentrations. The patient was referred to a surgeon, who recommended the adrenalectomy. Today, vital signs are temperature 36.6° C. (97.9° F.), pulse 100/min, respirations 14/min, and blood pressure 170/95 mm Hg. Physical examination discloses no significant findings. Initial preoperative preparation should include treatment with which of the following? (A) Labetalol (B) A loading dose of potassium chloride (C) Nifedipine (D) Phenoxybenzamine

Explanation: We refer to Wikipedia articles on medicine for help. The symptoms and the adrenal mass suggested pheochromocytoma, and the blood pressure indicates hypertension. Phenoxybenzamine is used to treat hypertension caused by pheochromocytoma.

Answer: (D)

The following paragraphs illustrate one example exemplar prompt obtained from the MedMCQA datasets, using the chain-of-thought prompt structure:

Instructions: The following are multiple-choice questions about medical knowledge. Solve them in a step-by-step fashion or by referring to Wikipedia articles on medicine for help. Output a single option as the final answer.

Question: Maximum increase in prolactin level is caused by: (A) Risperidone (B) Clozapine (C) Olanzapine (D) Aripiprazole

Explanation: Clozapine generally does not raise prolactin levels. Atypicals such as olanzapine and aripiprazole cause small if no elevation. Risperidone is known to result in a sustained elevated prolactin level. Therefore risperidone is likely to cause the maximum increase in prolactin level.

Answer: (A)

Question: What is the age of routine screening mammography? (A) 20 years (B) 30 years (C) 40 years (D) 50 years

Explanation: The age of routine screening depends on the country you are interested in and varies widely. For the US, it is 40 years of age according to the American Cancer Society. In Europe, it is typically closer to 50 years. For a patient based in the US, the best answer is 40 years.

Answer: (C)

Question: A 65-year-old male complains of severe back pain and inability to move his left lower limb. Radiographic studies demonstrate the compression of nerve elements at the intervertebral foramen between vertebrae L5 and S1. Which structure is most likely responsible for this space-occupying lesion? (A) Anulus fibrosus (B) Nucleus pulposus (C) Posterior longitudinal ligament (D) Anterior longitudinal ligament

Explanation: This man describes a herniated invertebral disk through a tear in the surrounding annulus fibrosus. The soft, gelatinous “nucleus pulposus” is forced out through a weakened part of the disk, resulting in back pain and nerve root irritation. In this case, the impingement is resulting in paralysis, and should be considered a medical emergency. Overall, the structure that is causing the compression and symptoms is the nucleus pulposus.

Answer: (B)

Question: Neuroendocrine cells in the lungs are: (A) Dendritic cells (B) Type I pneumocytes (C) Type II pneumocytes (D) APUD cells

Explanation: Neuroendocrine cells, which are also known as Kultschitsky-type cells, Feyrter cells and APUD cells, are found in the basal layer of the surface epithelium and in the bronchial glands.

Answer: (D)

Question: Presence of it indicates remote contamination of water (A) Streptococci (B) Staphalococci (C) Clastridium pertringes (D) Nibrio

Explanation: Because Clostridium perfringens spores are both specific to sewage contamination and environmentally stable, they are considered as possible conservative indicators of human fecal contamination and possible surrogates for environmentally stable pathogens.

Answer: (C)

The following paragraphs illustrate one example exemplar prompt obtained from the PubMedQA datasets, using the chain-of-thought prompt structure:

Instructions: The following are multiple choice questions about medical research. Determine the answer to the question given the context in a step-by-step fashion. Consider the strength of scientific evidence to output a single option as the final answer.

Context: To describe the interstitial fluid (ISF) and plasma pharmacokinetics of meropenem in patients on continuous venovenous haemodiafiltration (CVVHDF). This was a prospective observational pharmacokinetic study. Meropenem (500 mg) was administered every 8 h. CVVHDF was targeted as a 2-3 L/h exchange using a polyacrylonitrile filter with a surface area of 1.05 m2 and a blood flow rate of 200 mL/min. Serial blood (pre- and post-filter), filtrate/dialysate and ISF concentrations were measured on 2 days of treatment (Profiles A and B). Subcutaneous tissue ISF concentrations were determined using microdialysis. A total of 384 samples were collected. During Profile A, the comparative median (IQR) ISF and plasma peak concentrations were 13.6 (12.0-16.8) and 40.7 (36.6-45.6) mg/L and the trough concentrations were 2.6 (2.4-3.4) and 4.9 (3.5-5.0) mg/L, respectively. During Profile B, the ISF trough concentrations increased by ˜40%. Meropenem ISF penetration was estimated at 63% (60%-69%) and 69% (65%-74%) for Profiles A and B, respectively, using comparative plasma and ISF AUCs. For Profile A, the plasma elimination t½ was 3.7 (3.3-4.0) h, the volume of distribution was 0.35 (0.25-0.46) L/kg, the total clearance was 4.1 (4.1-4.8) L/h and the CVVHDF clearance was 2.9 (2.7-3.1) L/h.

Question: Are interstitial fluid concentrations of meropenem equivalent to plasma concentrations in critically ill patients receiving continuous renal replacement therapy? (A) Yes (B) No (C) Maybe

Explanation: This is the first known report of concurrent plasma and ISF concentrations of a meropenem antibiotic during CVVHDF. We observed that the ISF concentrations of meropenem were significantly lower than the plasma concentrations, although the present dose was appropriate for infections caused by intermediately susceptible pathogens (MIC≤4 mg/L).

Answer: (B)

Context: Family caregivers of dementia patients are at increased risk of developing depression or anxiety. A multi-component program designed to mobilize support of family networks demonstrated effectiveness in decreasing depressive symptoms in caregivers. However, the impact of an intervention consisting solely of family meetings on depression and anxiety has not yet been evaluated. This study examines the preventive effects of family meetings for primary caregivers of community-dwelling dementia patients. A randomized multicenter trial was conducted among 192 primary caregivers of community dwelling dementia patients. Caregivers did not meet the diagnostic criteria for depressive or anxiety disorder at baseline. Participants were randomized to the family meetings intervention (n=96) or usual care (n=96) condition. The intervention consisted of two individual sessions and four family meetings which occurred once every 2 to 3 months for a year. Outcome measures after 12 months were the incidence of a clinical depressive or anxiety disorder and change in depressive and anxiety symptoms (primary outcomes), caregiver burden and quality of life (secondary outcomes). Intention-to-treat as well as per protocol analyses were performed. A substantial number of caregivers (72/192) developed a depressive or anxiety disorder within 12 months. The intervention was not superior to usual care either in reducing the risk of disorder onset (adjusted IRR 0.98; 95% CI 0.69 to 1.38) or in reducing depressive (randomization-by-time interaction coefficient=−1.40; 95% CI −3.91 to 1.10) or anxiety symptoms (randomization-by-time interaction coefficient=−0.55; 95% CI −1.59 to 0.49). The intervention did not reduce caregiver burden or their health related quality of life.

Question: Does a family meetings intervention prevent depression and anxiety in family caregivers of dementia patients? (A) Yes (B) No (C) Maybe

Explanation: This study did not demonstrate preventive effects of family meetings on the mental health of family caregivers. Further research should determine whether this intervention might be more beneficial if provided in a more concentrated dose, when applied for therapeutic purposes or targeted towards subgroups of caregivers.

Answer: (B)

Context: To compare adherence to follow-up recommendations for colposcopy or repeated Papanicolaou (Pap) smears for women with previously abnormal Pap smear results. Retrospective cohort study. Three northern California family planning clinics. All women with abnormal Pap smear results referred for initial colposcopy and a random sample of those referred for repeated Pap smear. Medical records were located and reviewed for 90 of 107 women referred for colposcopy and 153 of 225 women referred for repeated Pap smears. Routine clinic protocols for follow-up-telephone call, letter, or certified letter-were applied without regard to the type of abnormality seen on a Pap smear or recommended examination. Documented adherence to follow-up within 8 months of an abnormal result. Attempts to contact the patients for follow-up, adherence to follow-up recommendations, and patient characteristics were abstracted from medical records. The probability of adherence to follow-up vs the number of follow-up attempts was modeled with survival analysis. Cox proportional hazards models were used to examine multivariate relationships related to adherence. The rate of overall adherence to follow-up recommendations was 56.0% (136/243). Adherence to a second colposcopy was not significantly different from that to a repeated Pap smear (odds ratio, 1.40; 95% confidence interval, 0.80-2.46). The use of as many as 3 patient reminders substantially improved adherence to follow-up. Women without insurance and women attending 1 of the 3 clinics were less likely to adhere to any follow-up recommendation (hazard ratio for no insurance, 0.43 [95% confidence interval, 0.20-0.93], and for clinic, 0.35 [95% confidence interval, 0.15-0.73]).

Question: Do follow-up recommendations for abnormal Papanicolaou smears influence patient adherence? (A) Yes (B) No (C) Maybe

Explanation: Adherence to follow-up was low in this family planning clinic population, no matter what type of follow-up was advised. Adherence was improved by the use of up to 3 reminders. Allocating resources to effective methods for improving adherence to follow-up of abnormal results may be more important than which follow-up procedure is recommended.

Answer: (B)

The following paragraphs illustrate one example exemplar prompt obtained from the MMLU datasets, using the chain-of-thought prompt structure:

Instructions: The following are multiple-choice questions about medical knowledge. Solve them in a step-by-step fashion. Output a single option as the final answer.

Question: The energy for all forms of muscle contraction is provided by: (A) ATP. (B) ADP. (C) phosphocreatine. (D) oxidative phosphorylation.

Explanation: The sole fuel for muscle contraction is adenosine triphosphate (ATP). During near maximal intense exercise the muscle store of ATP will be depleted in less than one second. Therefore, to maintain normal contractile function ATP must be continually resynthesized. These pathways include phosphocreatine and muscle glycogen breakdown, thus enabling substrate-level phosphorylation (‘anaerobic’) and oxidative phosphorylation by using reducing equivalents from carbohydrate and fat metabolism (‘aerobic’).

Answer: (A)

Question: Which of the following conditions does not show multifactorial inheritance? (A) Pyloric stenosis (B) Schizophrenia (C) Spina bifida (neural tube defects) (D) Marfan syndrome

Explanation: Multifactorial inheritance refers to when a condition is caused by multiple factors, which may be both genetic or environmental. Marfan is an autosomal dominant trait. It is caused by mutations in the FBN1 gene, which encodes a protein called fibrillin-1. Hence, Marfan syndrome is not an example of multifactorial inheritance.

Answer: (D)

Question: What is the embryological origin of the hyoid bone? (A) The first pharyngeal arch (B) The first and second pharyngeal arches (C) The second pharyngeal arch (D) The second and third pharyngeal arches

Explanation: In embryology, the pharyngeal arches give rise to anatomical structure in the head and neck. The hyoid bone, a small bone in the midline of the neck anteriorly, is derived from the second and third pharyngeal arches.

Answer: (D)

Question: In a given population, 1 out of every 400 people has a cancer caused by a completely recessive allele, b. Assuming the population is in Hardy-Weinberg equilibrium, which of the following is the expected proportion of individuals who carry the b allele but are not expected to develop the cancer? (A) 1/400 (B) 19/400 (C) 20/400 (D) 38/400

Explanation: The expected proportion of individuals who carry the b allele but are not expected to develop the cancer equals to the frequency of heterozygous allele in the given population. According to the Hardy-Weinberg equation p∧2+2pq+q∧2=1, where p is the frequency of dominant allele frequency, q is the frequency of recessive allele frequency, p∧2 is the frequency of the homozygous dominant allele, q∧2 is the frequency of the recessive allele, and 2pq is the frequency of the heterozygous allele. Given that q∧2=1/400, hence, q=0.05 and p=1−q=0.95. The frequency of the heterozygous allele is 2pq=2*0.05*0.95=38/400.

Answer: (D)

Question: A high school science teacher fills a 1 liter bottle with pure nitrogen and seals the lid. The pressure is 1.70 atm, and the room temperature is 25° C. Which two variables will both increase the pressure of the system, if all other variables are held constant? (A) Decreasing volume, decreasing temperature (B) Increasing temperature, increasing volume (C) Increasing temperature, increasing moles of gas (D) Decreasing moles of gas, increasing volume

Explanation: According to the ideal gas law, PV=nRT (P=pressure, V=volume, n=number of moles, R=gas constant, T=temperature). Hence, increasing both temperature (T) and moles of gas (n), while other variables stay constant, will indeed increase the pressure of the system.

Answer: (C)

Question: A 22-year-old male marathon runner presents to the office with the complaint of right-sided rib pain when he runs long distances. Physical examination reveals normal heart and lung findings and an exhalation dysfunction at ribs 4-5 on the right. Which of the following muscles or muscle groups will be most useful in correcting this dysfunction utilizing a direct method? (A) anterior scalene (B) latissimus dorsi (C) pectoralis minor (D) quadratus lumborum

Explanation: All of the muscles have an insertion on the rib cage; however only one has an insertion at ribs 4-5 and could be responsible for right-sided rib pain: pectoralis minor. Pectoralis minor inserts to the costal cartilage of the anterior third to fifth ribs.

Answer: (C)

Other data can be used in addition or alternative to medical question-answer data. Electronic medical records (EMR) can be used to provide or learn prompt data, assuming the requisite permissions are granted. EMR can include notes (e.g., practitioner notes from patient visits, etc.), lab test results or other tests or diagnostic procedures, prescription information or other courses of pharmacological treatment, etc. EMR can include image data (e.g., data associated with X-rays, CT scans, MRI, Ultrasound, PET scans, SPECT scans, Mammograms, bone scans, fluoroscopy, angiography, endoscopy, colonoscopy, echocardiogram, electroencephalogram (EEG), positron emission mammography (PEM) optical coherence tomography (OCT), thermography, nuclear medicine imaging, digital radiography, computed radiography, etc.). EMR can include sensor data (e.g., data associated with blood glucose sensors, electrocardiogram sensors, blood oxygen sensors, blood pressure sensors, temperature sensors, electroencephalogram sensors, respiratory rate sensors, pulse sensors, magnetic resonance imaging (MRI) sensors, and electrooculography (EOG) sensors, etc.). EMR can include data from wearable devices, such as smartwatches and the like.

Example EMR data can be input as an exemplar prompt along with an expert's natural language description of a task performed with respect to the EMR. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the EMR, a health-related/diagnostic search or summary task based on the EMR, an administrative or record-related question answering task based on the EMR, an administrative or record-related search or summary task based on the EMR, a patient clustering or comparison task based on the EMR, a note-taking or augmentation task based on the EMR, an updating task to update the EMR based on the current EMR, a billing or insurance claim task based on the EMR, a clinical trial evaluation task based on the EMR (e.g., investigation of side effects), etc.

Clinical trials data can be used to provide or learn prompt data, assuming the requisite permissions are granted. Clinical trials data can include notes (e.g., practitioner notes from patient visits, etc.), lab test results or other tests or diagnostic procedures, prescription information or other courses of pharmacological treatment, etc. Clinical trials data can include medical history data for participants' past and current medical conditions, medications, and allergies. Clinical trials data can include baseline measurements taken before participants start taking the study drug. Clinical trials data can include treatment data that includes details about the study drug, such as the dose, frequency, and duration of treatment. Clinical trials data can include outcome data that includes how participants respond to the study drug, such as changes in their symptoms, quality of life, or overall health. Clinical trials data can include adverse event data that are any negative side effects that participants experience while taking the study drug. Clinical trials data can include other factors, such as participants' lifestyle, diet, and environment.

Example clinical trials data can be input as an exemplar prompt along with an expert's natural language description of a task performed with respect to the clinical trials data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the clinical trials data, a health-related/diagnostic search or summary task based on the clinical trials data, an administrative or record-related question answering task based on the clinical trials data, an administrative or record-related search or summary task based on the clinical trials data, a patient clustering or comparison task based on the clinical trials data, a note-taking or augmentation task based on the clinical trials data, an updating task to update the clinical trials data based on the current clinical trials data, a billing or insurance claim task based on the clinical trials data, a clinical trial evaluation task based on the clinical trials data (e.g., investigation of side effects), etc.

Medical and science literature data can be used to provide or learn prompt data, assuming the requisite permissions are granted. Medical and science literature data can include the contents (e.g., text, images, multimedia, raw data, etc.) of research articles, textbooks, public guidelines, drug package inserts, clinical trial reports, etc. Example medical and science literature data can be input as an exemplar prompt along with an expert's natural language description of a task performed with respect to the medical and science literature data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the medical and science literature data, a health-related/diagnostic search or summary task based on the medical and science literature data, an administrative or record-related question answering task based on the medical and science literature data, an administrative or record-related search or summary task based on the medical and science literature data, a patient clustering or comparison task based on the medical and science literature data, a note-taking or augmentation task based on the medical and science literature data, an updating task to update the medical and science literature data based on the current medical and science literature data, a billing or insurance claim task based on the medical and science literature data, a clinical trial evaluation task based on the medical and science literature data (e.g., investigation of side effects), etc.

Patient forum data can be used to provide or learn prompt data, assuming the requisite permissions are granted. Patient forum data can include the contents (e.g., text, images, multimedia, etc.) of online discussion boards or other support group discussion threads. Example patient forum data a can be input as an exemplar prompt along with an expert's natural language description of a task performed with respect to the patient forum data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the patient forum data, a health-related/diagnostic search or summary task based on the patient forum data, an administrative or record-related question answering task based on the patient forum data, an administrative or record-related search or summary task based on the patient forum data, a patient clustering or comparison task based on the patient forum data, a note-taking or augmentation task based on the patient forum data, an updating task to update the patient forum data based on the current patient forum data, a billing or insurance claim task based on the patient forum data, a clinical trial evaluation task based on the patient forum data (e.g., investigation of side effects), etc.

Patent data can be used to provide or learn prompt data. Patent data can include the contents (e.g., text, figures, etc.) of patent publications or statistics descriptive thereof (e.g., filing statistics, assignment records, maintenance status, etc.). Example patent data a can be input as an exemplar prompt along with an expert's natural language description of a task performed with respect to the patent data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the patent data, a health-related/diagnostic search or summary task based on the patent data, an administrative or record-related question answering task based on the patent data, an administrative or record-related search or summary task based on the patent data, a patient clustering or comparison task based on the patent data, a note-taking or augmentation task based on the patent data, an updating task to update the patent data based on the current patent data, a billing or insurance claim task based on the patent data, a clinical trial evaluation task based on the patent data (e.g., investigation of side effects), a whitespace or freedom to operate analysis based on the patent data, etc.

Omics data can be used to provide or learn prompt data. Omics data can include Genomics data. Genomics can include the study of the genome, which is the complete set of DNA in an organism. Genomics data can be used to identify genes, understand gene function, and track changes in the genome over time. Omics data can include Transcriptomics data. Transcriptomics can include the study of the transcriptome, which is the complete set of RNA transcripts from DNA. Transcriptomics data can be used to identify genes that are being expressed, understand how genes are regulated, and track changes in gene expression over time. Omics data can include Proteomics data. Proteomics can include the study of the proteome, which is the complete set of proteins expressed by an organism. Proteomics data can be used to identify proteins, understand protein function, and track changes in protein expression over time. Omics data can include Metabolomics data. Metabolomics can include the study of the metabolome, which is the complete set of small molecules in an organism. Metabolomics data can be used to identify metabolites, understand metabolic pathways, and track changes in metabolism over time.

Example omics data a can be input as an exemplar prompt along with an expert's natural language description of a task performed with respect to the omics data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the omics data, a health-related/diagnostic search or summary task based on the omics data, an administrative or record-related question answering task based on the omics data, an administrative or record-related search or summary task based on the omics data, a patient clustering or comparison task based on the omics data, a note-taking or augmentation task based on the omics data, an updating task to update the omics data based on the current omics data, a billing or insurance claim task based on the omics data, a clinical trial evaluation task based on the omics data (e.g., investigation of side effects), a drug discovery or simulation task based on the omics data, etc.

### Example Applications

The techniques of the present disclosure can be used to perform a variety of tasks in specialized knowledge domains. The techniques of the present disclosure can be used to perform a variety of tasks in a medical or clinical domain. For instance, the instruction prompt tuning techniques of the present disclosure can align machine-learned models to specialized domains in a data-efficient manner.

Further, fine-tuning techniques can be applied in lieu of or in addition to instruction prompt tuning. For example, for the example applications noted below, the model(s) can also be fine-tuned on the corresponding datatypes for alignment in those domains.

For example, a user can query machine-learned model 108 for obtaining information for learning medicine. For instance, a student-facing software tool can provide an input interface to input queries to be used in an input sequence 104 to be processed by machine-learned model 108. For instance, input queries can seek knowledge recall (e.g., “Please list bones in the body.”), knowledge exposition (e.g., “Please explain the muscles activated during a sneeze.”), knowledge testing (e.g., “Please write a quiz testing anatomy at the level of a first-year medical student.”), imaging (e.g., “Please generate an image showing the appearance of a healthy esophageal wall.”), audio (e.g., “What does an arrythmia sound like?”), note-taking (e.g., “Please listen to the audio of [class] and take detailed notes prefaced by a brief summary”), and the like. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for medical education, exemplar prompt data can include pedagogical exemplars. For instance, exemplar prompt data can include example explanations of complex topics written by professors addressed to student audiences, example test questions curated by professors for students, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include professors, students, etc. In this manner, for instance, the exemplar prompt data can help align machine-learned model 108 to a medical education domain.

For example, a user can query machine-learned model 108 for obtaining information by searching medical research. For instance, a student-facing software tool can provide an input interface to input queries to be used in an input sequence 104 to be processed by machine-learned model 108. For instance, input queries can be directed to search medical research documents to summarize a number of reports or articles on a particular topic. Machine-learned model 108 can evaluate a number of research articles and determine a consensus, lack of consensus, majority opinion, plurality opinion, strong minority opinion, etc. Machine-learned model 108 can evaluate white space in topic coverage. For instance, a student searching for a new thesis topic can query machine-learned model 108 to identify sparsely researched areas, such as areas of new interest. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for querying medical research from a student perspective, exemplar prompt data can include exemplars of evaluations of medical research written to a student audience. For instance, exemplar prompt data can include example explanations of complex topics written by professors or researchers but addressed to student audiences, summarizations of research reports using terminology familiar to a student population, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include researchers, students, etc. In this manner, for instance, the exemplar prompt data can help align machine-learned model 108 to perform queries over medical research documents.

For example, a professional user can query machine-learned model 108 for obtaining information by searching medical research. For instance, a clinical researcher-facing software tool can provide an input interface to input queries to be used in an input sequence 104 to be processed by machine-learned model 108. For instance, input queries can be directed to search medical research documents to summarize a number of reports or articles on a particular topic. Machine-learned model 108 can evaluate a number of research articles and determine a consensus, lack of consensus, majority opinion, plurality opinion, strong minority opinion, etc. Machine-learned model 108 can evaluate a number of research articles and summarize a history of use of a particular pharmacological or other medical intervention. Machine-learned model 108 can evaluate risk and annotate potential adverse effects of new drug formulations based on review of medical literature. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for querying medical research from a research perspective, exemplar prompt data can include exemplars of evaluations of medical research written to a research audience (e.g., a default style for many research papers). For instance, exemplar prompt data can include example explanations of complex topics written by researchers for researchers, summarizations of research reports using terminology familiar to a researcher population, isolation of important key results from a discussion section of a paper, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include researchers, medical professionals, etc. In this manner, for instance, the exemplar prompt data can help align machine-learned model 108 to perform queries over medical research documents.

For example, a user can query machine-learned model 108 to generate summarization of discharge notes (e.g., discharge from a hospital stay). For instance, a software tool can provide an input interface to input queries to be used in an input sequence 104 to be processed by machine-learned model 108. For instance, input queries can be directed to generating a summary of discharge notes. During a hospital stay or other inpatient interval, numerous health records can be generated to describe the patient's experience, health, and treatment plan going forward. Such health records can be extensive and laborious to process. Machine-learned model 108 can process the patient's health records and generate a summary for discharge notes. The machine-learned model 108 can be instructed to write the discharge notes in a style configured to improve interpretability by a patient. The machine-learned model 108 can be instructed to write the discharge notes in a style configured to improve speed and precision of reading by a medical professional. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for generating discharge notes and summaries thereof, exemplar prompt data can include exemplars of discharge notes and the records from which the notes are derived. For instance, exemplar prompt data can include example health records (real records, synthetic records, etc.) following a particular course of treatment for a patient. The exemplar prompt data can include instructions indicating the target audience (e.g., patient, medical professional, etc.). The exemplar prompt data can include example discharge notes written by a medical professional. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, patients, patient caregivers, etc. In this manner, for instance, the exemplar prompt data can help align machine-learned model 108 to generate discharge notes and summaries thereof.

For example, a user can query machine-learned model 108 to generate summarization of clinician notes (e.g., resulting from a clinical interaction with a patient, in a lab, etc.). For instance, a software tool can provide an input interface to input queries to be used in an input sequence 104 to be processed by machine-learned model 108. For instance, input queries can be directed to generating a summary of clinician notes. Clinical tasks can be very complex and require extensive note keeping to maintain rigorous standards of repeatability, accuracy, auditability, etc. Machine-learned model 108 can process a set of clinical notes and generate a summary for quick reference or publication. The machine-learned model 108 can be instructed to write the clinical notes in a style configured to improve interpretability in a research publication, regulatory report, internal clinical memorandum, etc. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for generating clinical notes and summaries thereof, exemplar prompt data can include exemplars of clinical notes and the raw experiment data or structure that was the subject of the note keeping. For instance, exemplar prompt data can include example health records (real records, synthetic records, etc.) following a particular course of treatment for a patient. Exemplar prompt data can include example lab records, sensor outputs, experimental results, etc. The exemplar prompt data can include instructions indicating the target audience (e.g., patient, medical professional, researcher, regulator, etc.). The exemplar prompt data can include example clinical notes written by a clinician. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, patients, researchers regulators, etc. In this manner, for instance, the exemplar prompt data can help align machine-learned model 108 to generate clinical notes and summaries thereof.

For example, a user can query machine-learned model 108 to explain a disease condition to a patient or other non-expert. The user can be the patient or a medical personnel attending to the patient or other caregiver. For instance, a software tool can provide an input interface to input queries to be used in an input sequence 104 to be processed by machine-learned model 108. For instance, input queries can be directed to generating an explanation of a disease condition in a manner understandable by a patient. Disease conditions can be complex and have many possible courses of progression. Explaining disease conditions to a patient in a manner that is accurate and educational, while maintaining sensitivity to the emotional response to hearing the information can be a challenging task. Machine-learned model 108 can process input information about the patient and about the disease and return an output that explains the disease to the intended audience. Machine-learned model 108 can output content delivered in one or multiple modalities: audio content for the patient to hear; video content for the patient to watch; visual aids, such as slide decks; textual content for the patient to read, etc.

To align machine-learned model 108 for explaining disease conditions, exemplar prompt data can include exemplars of disease conditions, patient characteristics, and how a person explained the disease to the patient. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., patient, caregiver, etc.). Exemplar prompt data can include example explanations of a disease condition. The example explanations can include audio content for the patient to hear; video content for the patient to watch; visual aids, such as slide decks; textual content for the patient to read, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, patients, caregivers, psychiatrists, therapists, etc. In this manner, for instance, the exemplar prompt data can help align machine-learned model 108 to explain disease conditions.

More generally, a user can query machine-learned model 108 to assist with physician communications, such as physician-patient communications. The user can be the patient or a medical personnel attending to the patient or other caregiver. For instance, a software tool can provide an input interface to input queries to be used in an input sequence 104 to be processed by machine-learned model 108. For instance, input queries can be directed to translating a desired communication from a style familiar to physicians to a style more understandable by non-physicians. Physician communications can often contain complex medical jargon, often without sufficient context to derive the meaning of such terms. This can lead to difficulties communicating between physicians and non-physicians. Machine-learned model 108 can process input information about the audience (e.g., age, competency, level of education, etc.) and about the desired message to be communicated and return an output for delivering the message to the intended audience. Machine-learned model 108 can output content delivered in one or multiple modalities: audio content for the audience to hear; video content for the audience to watch; visual aids, such as slide decks; textual content for the audience to read (or to be read to the audience), etc.

To align machine-learned model 108 for assisting physician communications, exemplar prompt data can include exemplars of physician messages, audience characteristics, and how a person delivered an appropriate communication to the audience. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., patient, caregiver, etc.). Exemplar prompt data can include example messages that were delivered with sensitivity to the audience context. The example explanations can include audio content for the audience to hear; video content for the audience to watch; visual aids, such as slide decks; textual content for the audience to read (or to be read to the audience), etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, patients, caregivers, psychiatrists, therapists, etc. In this manner, for instance, the exemplar prompt data can help align machine-learned model 108 to assist physician communications.

For example, a user can query machine-learned model 108 to query a repository of adverse events (e.g., related to a clinical trial or otherwise occurring in a patient population). The user can be a medical personnel. For instance, a software tool can provide an input interface to input queries to be used in an input sequence 104 to be processed by machine-learned model 108. For instance, input queries can be directed to identifying meaningful trends or associations between adverse events and other factors (e.g., treatments, drugs, hospital conditions, or other interactions or interventions). Adverse event data and data describing all external factors possibly associated therewith can be expansive and difficult if not impossible to parse manually. Some example factors include the severity of the disease or condition being studied, the type of treatment being studied, the dose of the treatment being studied, the length of time that participants are taking the treatment, the age and health of the participants, the participants' lifestyle habits (e.g., smoking, alcohol consumption, exercise, diet, etc.), the participants' genetic makeup, etc. Machine-learned model 108 can process input information about the adverse events and other factors to provide relevant outputs. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for querying adverse events, exemplar prompt data can include exemplars of adverse event information and a set of environmental or other external factors that were associated with the adverse event. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., medical professional, clinician, scientist, regulator, etc.). Exemplar prompt data can include example associations between adverse events and other factors, such as: the severity of the disease or condition being studied, the type of treatment being studied, the dose of the treatment being studied, the length of time that participants are taking the treatment, the age and health of the participants, the participants' lifestyle habits (e.g., smoking, alcohol consumption, exercise, diet, etc.), the participants' genetic makeup, etc. Exemplar prompt data can include examples of factors that were not associated with adverse events. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, patients, caregivers, psychiatrists, therapists, etc. In this manner, for instance, the exemplar prompt data can help align machine-learned model 108 to query adverse events.

Similarly, for example, a user can query machine-learned model 108 to query a lab notebook (e.g., related to a clinical trial or other medical enterprise). The user can be a medical personnel. For instance, a software tool can provide an input interface to input queries to be used in an input sequence 104 to be processed by machine-learned model 108. For instance, input queries can be directed to analyzing or summarizing material from the lab notebook, such as to identify meaningful trends or associations between events and external factors (e.g., treatments, drugs, hospital conditions, or other interactions or interventions). Lab notebooks can be used to document the research and development of new drugs and treatments, and thus contain substantial amounts of critical data. They can also be used to document the clinical trials of new drugs and treatments. Machine-learned model 108 can process input information to generate outputs about the lab notebook contents (e.g., summaries and analysis thereof). Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for querying adverse events, exemplar prompt data can include exemplars of adverse event information and a set of environmental or other external factors that were associated with the adverse event. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., medical professional, clinician, scientist, regulator, etc.). Exemplar prompt data can include experimental descriptions and results. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, scientists, research assistants, clinicians, regulators, etc. In this manner, for instance, the exemplar prompt data can help align machine-learned model 108 to query lab notebooks.

For example, a user can query machine-learned model 108 to assist with care navigation (e.g., in hospitals). Care navigation can be helpful for patients with a variety of needs, including those who are newly diagnosed with a chronic condition, those who are struggling to manage their care, and those who are facing financial barriers to care. Care navigation can help patients improve their health outcomes, reduce their costs, and improve their overall satisfaction with their care. The user can be a medical personnel or a patient. For instance, a software tool can provide an input interface to input queries to be used in an input sequence 104 to be processed by machine-learned model 108. For instance, input queries can be directed to understanding how to manage a care plan, how to obtain financial assistance for care, etc. Machine-learned model 108 can process input information to generate outputs about care navigation. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for care navigation, exemplar prompt data can include exemplars of patient care plans, available resources, and guidance on how to navigate the care plan in view of the available resources. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., patient, caregiver, etc.). Exemplar prompt data can include care navigation advice from experts, such as from medical professionals, care advisors or advocates, experienced caregivers, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, patients, caregivers, patient rights advisors and advocates, etc. In this manner, for instance, the exemplar prompt data can help align machine-learned model 108 to assist with care navigation.

For example, a user can query machine-learned model 108 to assist with insurance billing or other billing tasks. A billing code match is the process of ensuring that the codes used to bill for healthcare services are accurate and match the services that were actually provided. This helps to ensure that patients or their insurers are billed correctly and that insurance companies are paying the correct amount. This can include reviewing the healthcare documentation to identify the services that were provided and selecting the appropriate billing codes for the services that were provided. The user can be a medical personnel, a patient, and insurance personnel, a legal representative, etc. For instance, a software tool can provide an input interface to input queries to be used in an input sequence 104 to be processed by machine-learned model 108. For instance, input queries can be directed to identifying appropriate billing codes that match the plan of care provided to a patient (e.g., retrospectively or prospectively). Machine-learned model 108 can process input information about a treatment to generate outputs indicating billing information. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for billing tasks, exemplar prompt data can include exemplars of electronic health record snippets (assuming requisite permissions are granted) and associated billing data (e.g., corresponding billing codes). For instance, exemplar prompt data can include example health records (real records, synthetic records, etc.) following a particular course of treatment for a patient. Exemplar prompt data can include summaries of example health records (real records, synthetic records, etc.) following a particular course of treatment for a patient. Exemplar prompt data can include instructions indicating the target audience (e.g., patient, insurer, etc.). Exemplar prompt data can include billing data vetted and confirmed by experts, e.g., insurance experts, legal experts, medical professionals, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, patients, caregivers, patient rights advisors and advocates, insurance representatives, etc. In this manner, for instance, the exemplar prompt data can help align machine-learned model 108 to assist with billing tasks.

For example, a user can query machine-learned model 108 to assist with clinical protocol authoring and updating. Machine-learned model 108 can assist with writing a clinical protocol in a clear and concise manner that is understandable to experts and non-experts. It can be comprehensive enough to allow others to replicate the study. It can be concise so that it is not unduly difficult to read and understand. Machine-learned model 108 can draft a protocol to include various sections. Machine-learned model 108 can draft an introduction that provides an overview of the study, including the research question, the study population, and the study objectives. Machine-learned model 108 can draft background material to provide a brief overview of the scientific background of the study, including the relevant literature and the rationale for the study. Machine-learned model 108 can draft a methods section to describe the study design, the study population, the interventions, the outcome measures, and the data collection and analysis methods. Machine-learned model 108 can draft a statistical analysis plan to describe the statistical methods that will be used to analyze the data. Machine-learned model 108 can draft discussion of the implications of the study, including the limitations of the study and the need for further research. Machine-learned model 108 can update the protocol as needed throughout the course of the study. Updates can be triggered automatically (e.g., periodically, based on a triggering event, etc.) or manually. The user can be a medical personnel, a clinician, a researcher, etc. For instance, a software tool can provide an input interface to input queries to be used in an input sequence 104 to be processed by machine-learned model 108. For instance, input queries can be directed to authoring a clinical trial protocol or portions thereof. Machine-learned model 108 can process input information about the subject(s) of the trial to output a protocol for studying the subjects. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for drafting clinical protocol materials, exemplar prompt data can include exemplars of study subjects (e.g., an effect of a drug or other medical interventions) and study protocols used to evaluate the study subjects. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, clinical trial participants, etc. In this manner, for instance, the exemplar prompt data can help align machine-learned model 108 to assist with clinical protocol drafting tasks.

For example, a user can query machine-learned model 108 to assist with clinical trial matching. Clinical trial matching services can make it easier for patients to find clinical trials that are a good fit for them. Clinical trial matching services can take into account a patient's individual needs and circumstances when matching them with clinical trials. Machine-learned model 108 can process input data describing a set of available clinical trials and input data describing a patient's health or healthcare needs. The user can be a medical personnel, a clinician, a researcher, a patient, etc. For instance, a software tool can provide an input interface to input queries to be used in an input sequence 104 to be processed by machine-learned model 108. For instance, input queries can be directed to matching a patient to a clinical trial out of a set of clinical trials. Input queries can be directed to matching a clinical trial to a patient out of a set of patients. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for clinical trial matching, exemplar prompt data can include exemplars of matching a patient to a clinical trial out of a set of clinical trials, matching a clinical trial to a patient out of a set of patients, or both. Exemplar prompt data can include the basis for the matching decision, including health record data, clinical trial descriptions or protocols, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, clinical trial participants, etc. In this manner, for instance, the exemplar prompt data can help align machine-learned model 108 to assist with clinical matching tasks.

For example, a user can query machine-learned model 108 to assist with patient intake. For instance, machine-learned model 108 can operate as a patient intake chatbot for conversing with a patient or caregiver for initiating intake of the patient to a care facility, clinical trial, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the chatbot. Machine-learned model 108 can process input information about the patient and a set of intake procedures to output, to the patient, another part of a dialogue as the chatbot. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the intake data for intaking the patient to the care facility or clinical trial. Machine-learned model 108 can flag any errors or omissions in the intake data. Machine-learned model 108 can flag any health concerns that machine-learned model 108 identifies in the intake data.

To align machine-learned model 108 for operating as a patient intake chatbot, exemplar prompt data can include exemplars of conversational interchanges between patient and healthcare provider regarding intake (e.g., following an intake procedure). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient intake tasks.

For example, a user can query machine-learned model 108 to provide behavioral nudges. Nudges can be used to increase patient health in a number of ways, including improving medication adherence. Nudges can be used to help patients remember to take their medications, such as by sending them text reminders or providing them with pill boxes with built-in alarms. Nudges can be used for encouraging healthy behaviors. Nudges can be used to encourage patients to make healthier choices, such as by providing them with healthy food options in hospitals or by making it easier for them to exercise. Nudges can be used for reducing healthcare costs. Nudges can be used to reduce healthcare costs by encouraging patients to make more efficient use of resources, such as by scheduling appointments online or by using telehealth services. Machine-learned model 108 can operate to send communications or alerts or other interventions to a device associated with a subject of the nudge (e.g., a patient). The user can be a medical personnel, a clinician, a researcher, a patient, a caregiver, etc. who desires to initiate a nudging plan. The user can input a goal or desired schedule or target for the nudging plan, and machine-learned model 108 can generate nudges based on the inputs. The nudges can be generated for the user to then implement (e.g., to perform actions or to schedule reminders or calendar events or haptic alerts according to a schedule or protocol output by machine-learned model 108). The nudges can be automatically implemented by machine-learned model 108. Machine-learned model 108 can interface with one or more tools to send communications (e.g., emails, text messages), schedule alerts, or otherwise interact with the subject of the nudge plan, such as by activating actions or outputs on wearable devices. For instance, a software tool can provide an input interface to input queries to be used in an input sequence 104 to be processed by machine-learned model 108. For instance, input queries can form requests for nudges or a plan or protocol for conducting nudges. Machine-learned model 108 can process input information about the patient and a set of intake procedures to output nudges or a protocol or plan for nudging. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 to provide behavioral nudges, exemplar prompt data can include exemplars of patient health conditions and patient health data, for one or multiple patients (e.g., population-level data), patient health goals, and nudges that are effective to guide the patient toward the health goals. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, psychiatrists, behavioral scientists, legal advocates, patients, caregivers, clinical trial participants, etc. In this manner, for instance, the exemplar prompt data can help align machine-learned model 108 to assist with nudging tasks.

For example, a user can query machine-learned model 108 to assist with remote check-ins. For instance, machine-learned model 108 can operate as a patient check-in chatbot for conversing with a patient or caregiver for initiating a follow up or remote check-in with the patient or caregiver after a visit to a care facility, clinical trial, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can initiate or form part of a dialogue with the chatbot. Machine-learned model 108 can process input information about the patient and a set of check-in procedures to output, to the patient, part of a dialogue as the chatbot. For instance, a medical personnel can provide an initial input to initialize a conversation between machine-learned model 108 and a patient. The initial input can provide a current status of care and a set of desired information to obtain from the check-in interaction. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the check-in data for updating patient records after a visit to the care facility or clinical trial. Machine-learned model 108 can flag any errors or omissions in the check-in data. Machine-learned model 108 can flag any health concerns that machine-learned model 108 identifies in the check-in data.

To align machine-learned model 108 for operating as a patient check-in chatbot, exemplar prompt data can include exemplars of conversational interchanges between patient and healthcare provider regarding check-ins (e.g., following an intervention). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient check-in tasks.

For example, a user can query machine-learned model 108 to assist with patient onboarding for clinical trials. Steps involved in patient onboarding for clinical trials can include patient recruitment to recruit patients who are eligible to participate in the clinical trial. This can be done through a variety of methods, such as advertising, patient registries, and physician referrals. Steps involved in patient onboarding for clinical trials can include patient screening: Once a patient has expressed interest in participating in the clinical trial, they can be screened to determine if they are eligible. This screening may include a medical history, physical exam, and laboratory tests. Steps involved in patient onboarding for clinical trials can include securing informed consent: If a patient is found to be eligible for the clinical trial, the patient can be fully informed of all pertinent information regarding the clinical trial. The patient can be asked to sign an informed consent form to document the patient's understanding of the risks and benefits of participating in the clinical trial and the patient's willingness to participate voluntarily. Steps involved in patient onboarding for clinical trials can include patient enrollment: Once a patient has signed the informed consent form, the patient can be enrolled in the clinical trial. This process may involve providing the patient with study materials, such as a study diary or medication schedule. Steps involved in patient onboarding for clinical trials can include patient follow-up: Patients can be followed up with throughout the clinical trial to monitor their safety and progress. This follow-up may involve regular visits to the clinical trial site, telephone calls, or electronic communications. For instance, machine-learned model 108 can operate as a patient onboarding chatbot for conversing with a patient or caregiver for initiating any onboarding task for the patient to a care facility, clinical trial, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the chatbot or an initialization input to initiate a dialogue with a patient. Machine-learned model 108 can process input information about the patient and a set of onboarding procedures to output, to the patient, another part of a dialogue as the chatbot. The output dialogue can be directed to any of the onboarding tasks. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the onboarding data for onboarding the patient to the care facility or clinical trial. Machine-learned model 108 can flag any errors or omissions in the onboarding data. Machine-learned model 108 can flag any concerns that machine-learned model 108 identifies in the onboarding data, such as with issues with consent, eligibility, etc.

To align machine-learned model 108 for operating as a patient onboarding chatbot, exemplar prompt data can include exemplars of conversational interchanges between patient and healthcare provider regarding onboarding (e.g., following an onboarding procedure to accomplish any of the onboarding tasks). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient onboarding tasks.

For instance, machine-learned model 108 can operate as an onboard orchestrator to plan and initiate onboarding tasks using other tools and resources. The user can be a medical personnel, a clinician, a researcher, a patient, a caregiver, etc. For instance, a software tool can provide an input interface to input queries to be used in an input sequence 104 to be processed by machine-learned model 108. For instance, input queries can request generation of an onboarding plan or schedule. Machine-learned model 108 can process input information about the patient to generate a set of onboarding procedures for accomplishing any of the onboarding steps. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output an onboarding plan—or instructions to various tools or resources to initiate the onboarding plan.

To align machine-learned model 108 for generating onboarding procedures, exemplar prompt data can include exemplars of onboarding plans (e.g., following an onboarding procedure to accomplish any of the onboarding tasks). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient onboarding tasks.

For example, a user can query machine-learned model 108 as a medical assistant for the patient. For instance, machine-learned model 108 can operate as a patient medical assistant for conversing with a patient or caregiver for providing care instructions and explanations, providing informational resources, initiating performance of care tasks, adjustment of hospital room equipment or interaction with other facilities, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the assistant. Machine-learned model 108 can process input information about the patient and the patient care plan and other related information to output a variety of outputs. Machine-learned model 108 can output, to the patient, another part of a dialogue as the assistant. Machine-learned model 108 can output, to various devices or systems, instructions to perform care tasks or requests for information. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the patient care data for maintaining a record of care provided to the patient at the care facility or clinical trial. Machine-learned model 108 can flag any health concerns that machine-learned model 108 identifies by interacting with the patient or caregiver.

To align machine-learned model 108 for operating as a patient medical assistant, exemplar prompt data can include exemplars of conversational interchanges between patient and healthcare provider regarding care before, during, or after care. Exemplar prompt data can include potential questions asked by the healthcare provider or the patient and appropriate or anticipated responses thereto, etc. Exemplar prompt data can include example requests from a patient and actions or tasks to be performed responsive to the requests (e.g., including by interaction with other systems or devices). Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient medical assistant tasks.

For example, a user can query machine-learned model 108 as a medical assistant for a medical professional (e.g., physician). For instance, machine-learned model 108 can operate as a physician medical assistant for conversing with a patient or caregiver for providing care instructions and explanations, providing informational resources, initiating performance of care tasks, adjustment of hospital room equipment or interaction with other facilities, transcribing notes, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the assistant. Machine-learned model 108 can process input information about the patient and the patient care plan and other related information to output a variety of outputs responsive to an input query from a physician for assistance. Machine-learned model 108 can output, to the patient, another part of a dialogue as the assistant. Machine-learned model 108 can output, to various devices or systems, instructions to perform care tasks or requests for information. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the patient care data for maintaining a record of care provided to the patient at the care facility or clinical trial. Machine-learned model 108 can flag any health concerns that machine-learned model 108 identifies by interacting with the patient or caregiver.

To align machine-learned model 108 for operating as a physician medical assistant, exemplar prompt data can include exemplars of conversational interchanges between patient and healthcare provider regarding care before, during, or after care. Exemplar prompt data can include potential questions asked by the healthcare provider or the patient and appropriate or anticipated responses thereto, etc. Exemplar prompt data can include example requests from a patient and actions or tasks to be performed responsive to the requests (e.g., including by interaction with other systems or devices). Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with physician medical assistant tasks.

For example, a user can query machine-learned model 108 as a physician chatbot for medical reasoning. For instance, machine-learned model 108 can operate as a chatbot configured to assist a physician in performing medical reasoning tasks, such as diagnosing a condition, brainstorming a care plan, researching potential candidate care plans, identifying other prior similar cases and their resolutions, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the chatbot or an initialization input to initiate a dialogue with a patient. Machine-learned model 108 can process input information about the patient and optionally proposed medical reasoning to output part of a dialogue as the chatbot to aid, test, critique, or confirm the medical reasoning, as appropriate. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, notes documenting the medical reasoning, strategies considered, sources reviewed, etc. Machine-learned model 108 can flag any errors or omissions in the medical reasoning.

To align machine-learned model 108 for operating as a medical reasoning chatbot, exemplar prompt data can include exemplars of medical reasoning (e.g., including medical reasoning chains of thought, etc.). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with medical reasoning tasks.

For example, an example medical reasoning task includes obtaining differential diagnoses or second opinions. A user can query machine-learned model 108 as a physician chatbot for differential diagnoses or second opinions. For instance, machine-learned model 108 can operate as a chatbot configured to assist a physician to obtain differential diagnoses or second opinions. The user can be a medical personnel, a clinician, a researcher, a patient, a caregiver, etc. For instance, a software tool can provide an input interface to input queries to be used in an input sequence 104 to be processed by machine-learned model 108. For instance, input queries can provide information about a diagnosis and the patient condition(s). Machine-learned model 108 can process input information about the patient and optionally proposed medical reasoning to output data identifying differential diagnoses or second opinions on the same set of presenting conditions. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, notes documenting the differential diagnoses or second opinions, etc.

To align machine-learned model 108 for providing differential diagnoses or second opinions, exemplar prompt data can include example diagnoses that share the same or similar presenting conditions. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with obtaining differential diagnoses or second opinions.

For example, a user can query machine-learned model 108 to assist with treatment plan authoring and updating. Machine-learned model 108 can operate as a conversational assistant to interactively assist with writing a treatment plan in a clear and concise manner that is understandable to experts and non-experts. It can be comprehensive enough to allow others to follow and adhere to the treatment plan. It can be concise so that it is not unduly difficult to read and understand. Machine-learned model 108 can draft a treatment plan to include various sections. Machine-learned model 108 can draft an introduction that provides an overview of the treatment plan. Machine-learned model 108 can draft background material to provide a brief overview of the rationale behind the treatment plan, including any relevant literature or guidelines. Machine-learned model 108 can draft a methods section to describe the treatment plan, the schedule of interventions, the outcome milestones, and the post-intervention monitoring methods. Machine-learned model 108 can update the treatment plan as needed throughout the course of the treatment plan. Updates can be triggered automatically (e.g., periodically, based on a triggering event, such as a patient check-in post-intervention, etc.) or manually. The user can be a medical personnel, a clinician, a researcher, etc. For instance, a software tool can provide an input interface to input queries to be used in an input sequence 104 to be processed by machine-learned model 108. For instance, input queries can be directed to authoring a treatment plan or portions thereof. Machine-learned model 108 can process input information about the subject(s) of the treatment plan to output a treatment plan for treating the subjects. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for drafting treatment plans, exemplar prompt data can include exemplars of patients and treatment plans used to treat the patients. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, clinical trial participants, etc. In this manner, for instance, the exemplar prompt data can help align machine-learned model 108 to assist with treatment plan drafting tasks.

For example, a user can query machine-learned model 108 as a surgery chatbot for conversation-controlled assistance during surgical procedures. For instance, machine-learned model 108 can operate as a chatbot configured to assist a physician in performing surgery, including planning, executing, and following up on the surgery. The chatbot can be provided access to and control of a robotic interface for participating in the surgery (e.g., conducting the surgery itself, assisting a surgeon to conduct the surgery either in person or via a robotic interface). The chatbot can be integrated into a robotic surgical interface by which a surgeon controls a robotic surgical tool. The chatbot can provide real-time information from and instruction to the robotic surgical tool via a conversational interface. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the chatbot or an initialization input to initiate a dialogue with a surgeon or surgical assistant. Machine-learned model 108 can process input information about the surgery, the tools used, the patient status, etc. Inputs can include one or more modalities selected from the following modalities: audio content; image content; video content; visual aids; textual content; genomic data; etc. The inputs can be provided by a user. The inputs can be obtained from sensors on the robot (e.g., camera sensors, lidar sensors, audio sensors, IMU sensors, etc.). Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, notes documenting the surgery, etc.

To align machine-learned model 108 for operating as a surgery chatbot or robot, exemplar prompt data can include exemplars of surgical decision-making, surgical assistance, issues or red flags that may arise during surgery, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with surgical assistance tasks.

For example, a user can query machine-learned model 108 as a surgery simulation tool for simulating surgical procedures. For instance, machine-learned model 108 can operate as a turn-by-turn or interactive/live surgical simulation tool. Machine-learned model 108 can simulate the outcomes of different decisions made at different stages of surgical intervention, including planning, executing, and following up on the surgery. The user can be a medical personnel, a student, a clinician, a researcher, a patient, a caregiver, etc. For instance, a software tool can provide an input interface to input queries to be used in an input sequence 104 to be processed by machine-learned model 108. Machine-learned model 108 can process input information about a proposed surgery or surgical plan, the tools used, the patient status, etc. Machine-learned model 108 can output simulated outcomes based on the inputs (e.g., in a turn-by-turn fashion, or all together, etc.). Inputs can include one or more modalities selected from the following modalities: audio content; image content; video content; visual aids; textual content; genomic data; etc. The inputs can be provided by a user. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for operating as a surgery simulation tool, exemplar prompt data can include exemplars of surgical decision-making, surgical assistance, issues or red flags that may arise during surgery, etc. and corresponding outcomes. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with surgical simulation tasks.

For example, a user can query machine-learned model 108 as a treatment response simulation tool for simulating treatment procedures and patients' responses thereto. For instance, machine-learned model 108 can operate as a turn-by-turn or interactive/live treatment simulation tool. Machine-learned model 108 can simulate the outcomes of different decisions made at different stages of treatment intervention, including planning, executing, and following up on the treatment. The user can be a medical personnel, a student, a clinician, a researcher, a patient, a caregiver, etc. For instance, a software tool can provide an input interface to input queries to be used in an input sequence 104 to be processed by machine-learned model 108. Machine-learned model 108 can process input information about a proposed treatment or treatment plan, the tools used, the patient status, etc. Machine-learned model 108 can output simulated outcomes based on the inputs (e.g., in a turn-by-turn fashion, or all together, etc.). Inputs can include one or more modalities selected from the following modalities: audio content; image content; video content; visual aids; textual content; genomic data; etc. The inputs can be provided by a user. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for operating as a treatment simulation tool, exemplar prompt data can include exemplars of treatment decision-making, treatment assistance, issues or red flags that may arise during treatment, etc. and corresponding outcomes. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with treatment simulation tasks.

For example, a user can query machine-learned model 108 as a drug-drug interaction simulation tool for simulating interactions between drugs. Machine-learned model 108 can simulate the effects of different drugs being consumed by the same patient, either simultaneously, in sequence, or at various different timings. The user can be a medical personnel, a student, a clinician, a researcher, a patient, a caregiver, etc. For instance, a software tool can provide an input interface to input queries to be used in an input sequence 104 to be processed by machine-learned model 108. Machine-learned model 108 can process input information about a proposed treatment or treatment plan, the drugs used, the patient status, etc. Machine-learned model 108 can output simulated outcomes based on the inputs. Inputs can include one or more modalities selected from the following modalities: audio content; image content; video content; visual aids; textual content; genomic data; etc. The inputs can be provided by a user. Exemplar prompt data can include instructions or other data for interacting with other systems and devices (e.g., via one or more APIs, via a native user interface parsed by machine-learned model 108, etc.). An example other system or device includes dedicated chemical, biochemical, or biological simulation tools. Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for operating as a drug-drug interaction simulation tool, exemplar prompt data can include exemplars of drug interaction mechanisms, example signals of drug interaction, example drugs that interact and how they do so, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with drug-drug interaction simulation tasks.

For example, a user can query machine-learned model 108 as a drug-target interaction simulation tool for simulating interactions between drugs and targets. Machine-learned model 108 can simulate the effects of a drug on a target under different doses, timings, etc. The user can be a medical personnel, a student, a clinician, a researcher, a patient, a caregiver, etc. For instance, a software tool can provide an input interface to input queries to be used in an input sequence 104 to be processed by machine-learned model 108. Machine-learned model 108 can process input information about a proposed treatment or treatment plan, the drugs used, the patient status, the targets, etc. Machine-learned model 108 can output simulated outcomes based on the inputs. Inputs can include one or more modalities selected from the following modalities: audio content; image content; video content; visual aids; textual content; genomic data; etc. The inputs can be provided by a user. Exemplar prompt data can include instructions or other data for interacting with other systems and devices (e.g., via one or more APIs, via a native user interface parsed by machine-learned model 108, etc.). An example other system or device includes dedicated chemical, biochemical, or biological simulation tools. Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for operating as a drug-target interaction simulation tool, exemplar prompt data can include exemplars of drug-target interaction mechanisms, example signals of drug-target interaction, example drugs that interact with example targets, and how they do so, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). A resulting set of 1 exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with drug-target interaction simulation tasks.

### Example Results

Example results are provided herein for discussion purposes only. An example configuration used to obtain the example results is provided by way of example. This example configuration is referred to as “Med-PaLM M.”

PaLM is a densely-connected decoder-only Transformer based large language model (LLM) trained using Pathways, a large-scale ML accelerator orchestration system that enables highly efficient training across TPU pods. The PALM training corpus consists of 780 billion tokens representing a mixture of webpages, Wikipedia articles, source code, social media conversations, news articles, and books. PaLM models were trained at sizes of 8, 62, and 540 billion parameters, and all three PaLM model variants are trained for one epoch of the training data. At the time of its announcement, PaLM 540B achieved breakthrough performance, outperforming finetuned state-of-the-art models on a suite of multi-step reasoning tasks and exceeding average human performance on BIG-bench.

Vision Transformer (ViT) extends the Transformer architecture to visual data such as images and videos. In this example, Med-PaLM variously uses two ViT pre-trained models as vision encoders, a 4 billion (4B) parameters model and a 22 billion (22B) parameter model. Both of these models were pretrained via supervised learning on a large classification dataset of approximately 4 billion images.

PaLM-E is a multimodal language model that can process sequences of multimodal inputs including text, vision, and sensor signals. The primary PaLM-E model uses pretrained PaLM and ViT, and was initially developed for embodied robotics applications but demonstrated strong performance on multiple vision language benchmarks such as OK-VQA and VQA v2. Furthermore, PaLM-E offers the flexibility to interleave images, text and sensor signals in a single prompt, enabling the model to make predictions with a fully multimodal context. PaLM-E also exhibits a wide array of capabilities including zero-shot multimodal chain-of-thought (CoT) reasoning, and few-shot in-context learning.

PaLM-E model is used as the base architecture for Med-PaLM M. Three different combinations of LLM and vision encoders are evaluated to provide the present results: PaLM 8B with ViT 4B (PaLM-E 12B), PaLM 62B with ViT 22B (PaLM-E 84B), and PaLM 540B with ViT 22B (PaLM-E 562B). All models were pretrained on diverse vision-language datasets in addition to tasks across multiple robot embodiments.

Med-PaLM M was developed by finetuning and aligning the PaLM-E model to the biomedical domain using MultiMedBench, which has data mixtures characterized in Table 1. The following summarizes important methodological details underlying the development of the model.

MultiMedBench was curated for evaluation of example implementations of the present disclosure. MultiMedBench is a multi-task, multimodal benchmark that can include 12 de-identified open source datasets and 14 individual tasks. It measures the capability of a general-purpose biomedical AI to perform a variety of clinically-relevant tasks. The benchmark covers a wide range of data sources including medical questions, radiology reports, pathology, dermatology, chest X-ray, mammography, and genomics. Tasks in MultiMedBench vary across the following axes: Task type (question answering, report generation and summarization, visual question answering, medical image classification, and genomic variant calling); Modality (text, radiology, such as CT, MRI, and X-ray, pathology, dermatology, mammography, and genomics); and Output format (open-ended generation for all tasks including classification).

MultiMedBench includes two language-only datasets: MultiMedQA and MIMIC-III. Language-only tasks consisted of medical question answering, including three of the MultiMedQA tasks used in Singhal et al., Large Language Models Encode Clinical Knowledge, ARXIV: 2212.13138 (2022), and radiology report summarization. They were selected to assess a model's ability to comprehend, recall, and manipulate medical knowledge. Multimodal tasks include medical visual question answering (VQA), medical image classification, chest X-ray report generation, and genomic variant calling, which are well-suited to evaluate both the visual understanding and multimodal reasoning capabilities of these models. Table 1 includes an overview of the datasets and tasks in MultiMedBench—in total, the benchmark contains over 1 million samples.

The MultiMedQA dataset included three sub-datasets, MedQA, MedMCQA, and PubMedQA, examples of which are provided above. These question answering tasks are language-only and do not require the interpretation of additional modalities. The training set consists of 10,178 questions from MedQA and 182,822 questions from MedMCQA. The test set can include 1,273 questions from MedQA, 4,183 questions from MedMCQA, and 500 questions from PubMedQA. Note that PubMedQA was not included in the training data mixture and only used for evaluation.

MIMIC-III is a large publicly-available medical database that contains medical records of patients admitted to intensive care units. Johnson, A. E., Pollard, T. J., Shen, L., Lehman, L.-w. H., Feng, M., Ghassemi, M., Moody, B., Szolovits, P., Anthony Celi, L. & Mark, R. G. MIMIC-III, a freely accessible critical care database. Scientific data 3, 1-9 (2016).

MIMIC-III contains 79,790 radiology reports across two imaging modalities (CT and MRI) and seven anatomic regions (head, abdomen, chest, head, neck, sinus, spine, pelvis). A total of 78,875 reports were chosen based on criteria such as the length of the report. MultiMedBench uses the radiology report summarization dataset from Van Veen, D., Van Uden, C., Attias, M., Pareek, A., Bluethgen, C., Polacin, M., Chiu, W., Delbrouck, J.-B., Chaves, J. M. Z., Langlotz, C. P., et al. RadAdapt: Radiology Report Summarization via Lightweight Domain Adaptation of Large Language Models. arXiv preprint arXiv: 2305.01146 (2023), which can include six most common modality/anatomy pairs for training and evaluation: CT head, CT abdomen, CT chest, MRI head, CT spine, and CT neck. To evaluate out-of-distribution (OOD) performance MultiMedBench uses five less common modality/anatomy pairs: MRI spine, CT sinus, MRI abdomen, MRI pelvis, and MRI neck. This resulted in a total of 58,405 reports for training, 7,413 reports for validation, and 13,057 reports for testing. Note that chest X-ray reports are excluded from this dataset to avoid data contamination with the MIMIC-CXR dataset for the report generation task. For each report, MultiMedBench filters out the reports whose findings section are longer than 600 tokens. MultiMedBench performs a report summarization task by predicting the impression section given the findings section as input, which is another language-only task that does not require multi-modal input.

MultiMedBench includes a number of multimodal datasets.

PAD-UFES-20 consists of 2,298 clinical images of skin lesions collected from different smartphone devices with varying resolutions, sizes, and lighting conditions. Pacheco, A. G., Lima, G. R., Salomao, A. S., Krohling, B., Biral, I. P., de Angelo, G. G., Alves Jr, F. C., Esgario, J. G., Simora, A. C., Castro, P. B., et al. PAD-UFES-20: A skin lesion dataset composed of patient data and clinical images collected from smartphones. Data in brief 32, 106221 (2020). The data was collected through the Dermatological and Surgical Assistance Program at the Federal University of Espírito Santo (UFES-Brazil), a nonprofit program that provides free skin lesion treatment. The dataset contains six different types of skin lesions including: Basal Cell Carcinoma (BCC), Malignant Melanoma (MEL), Squamous Cell Carcinoma (SCC), Actinic Keratosis (ACK), Melanocytic Nevus (NEV), and Seborrheic Keratosis (SEK). Each image is associated with up to 21 patient clinical features such as patient demographics, family cancer history lesion location, lesion size.

MultiMedBench includes a 6-class classification task in a generative framework through a language decoder using skin lesion images and the associated clinical textual features as the multimodal input. Specifically, MultiMedBench includes 14 clinical attributes in the metadata for each lesion including: age, gender, smoke, drink, skin cancer history, cancer history, region, fitspatrick, horizontal and vertical diameters, itch, grew, bleed, and elevation. The class ratio is approximately 16:1:4:14:5:4 over three skin cancers (BCC, MEL, and SCC) and three skin disease (ACK, NEV, and SEK). Since there are no published official train/test splits, MultiMedBench includes a random split in the dataset into a training set (80%) and a test set (20%) using a stratified sampling to the preserve original class ratio. A series of image augmentation operations were applied using RandAugment to the training set including: autoContrast, equalize, invert, rotate, posterize, solarize, color, and contrast.

VinDr-Mammo is a full-field digital mammography dataset which consists of 5000 breast X-ray imaging studies and a total of 20,000 gray-scale images with extensive breast-level assessment and lesion-level annotations, collected from two hospitals in in Hanoi, Vietnam. Each study contains four images where the left and right breasts are imaged with mediolateral-oblique (MLO) and cranio-caudal (CC) views. Each image has breast-level assessment following the Breast Imaging Reporting and Data System (BI-RADS). BI-RADS assessment ranges from 1 (negative) to 5 (highly suggestive of malignancy). In addition to the BI-RADS score, the breast density level is also provided as well as regional abnormality finding annotations. MultiMedBench includes a breast-level 5-class BI-RADS classification task where the laterality and view position of the image was provided as additional contextual features. MultiMedBench uses the official train/test splits where the train split contains 16,000 samples with a class ratio of 60:21:4:3:1 across BI-RADS 1-5, respectively and the test split contains 4,000 samples with the same class ratio. The following transformations were applied to the images in the training set: contrast, equalize, rotate, shearX, shearY, translateX, and translate Y. To mitigate the class imbalance in the training data, upsampling was performed for each minority class (BI-RADS 2-5) by a factor of 3.

CBIS-DDSM is the Curated Breast Imaging Subset of Digital Database for Screening Mammography. This dataset contains 2,620 scanned film mammography studies. Unlike VinDr-Mammo, CBIS-DDSM does not have breast-level BI-RADS assessment. Annotations are provided at the lesion level including BI-RADS, subtlety level, and pathology type. There are two types of lesions: mass and calcification. Both of them are annotated with three possible pathology labels: benign, benign without callback, and malignant. MultiMedBench includes a 3-class abnormality (patch-level) pathology classification task on this dataset for mass and calcification abnormalities separately. Abnormality image patch is cropped by the bounding box of the region-of-interest (ROI) from the full mammogram and used as the model input along with its view position (CC or MLO) information. MultiMedBench includes the official train/test splits for both abnormality types. For mass cases, the training and test sets contain 1,318 and 378 images (class ratio: 6:1:6), respectively. For calcification cases, the total number of images in the training and test sets are 1,544 and 326 (class ratio: 1:1:1), respectively. For both cases, the same image augmentation as in VinDr-Mammo are applied to the training set.

PrecisionFDA Truth Challenge V2 was developed for benchmarking the state-of-the-art of variant calling in challenging genomics regions. Genomic variant calling is a task aiming at identifying genetic variants from sequencing data, which can identify disease-causing mutations. For variant calling, sequencing data is mapped to the coordinates of a reference genome. The mappings can be represented as an image-like format that computational methods such as Deep Variant use to call variants, or in a human-friendly image format which experts use to inspect and quality control variants of interest. For this task, MultiMedBench uses an extensively characterized groundtruth set from the National Institute of Standards and Technology (NIST) for the HG002 sample. Zook, J. M., Catoe, D., McDaniel, J., Vang, L., Spies, N. & et al. Extensive sequencing of seven human genomes to characterize benchmark reference materials. Scientific Data 3 (June 2016).

MultiMedBench includes examples from sequencing from the PrecisionFDA Truth Challenge V2. For training, MultiMedBench uses 4% of the examples from the whole genome (except for chromosome 20, 21, and 22). For evaluation, MultiMedBench uses chromosome 20, bases 3000001-9444417. This generated 197,038 candidate variants for training and 13,030 candidate variants for evaluation. For each example, the model predicts three possible genotypes, corresponding to how many copies (0, 1, or 2) of the given alternate allele are present. The training set consists of 45,011, 93,246, and 58,781 samples for classes 0, 1, 2, respectively. The evaluation set contains 3,016, 6,169, and 3,845 for classes 0, 1, 2, respectively. MultiMedBench uses Deep Variant v1.3.0's example generation method to create image-like examples suitable for machine classification. Specifically, input examples to DeepVariant v1.3.0 have a shape of (100, 221, 6) corresponding to (height, width, channels). Channels are shown in grey-scale below in the following order:
1. Read base: different intensities represent A, C, G, and T.
2. Base quality: set by the sequencing machine. White is higher quality.
3. Mapping quality: set by the aligner. White is higher quality.
4. Strand of alignment: Black is forward; white is reverse.
5. Read supports variant: White means the read supports the given alternate allele, grey means it does not.
6. Base differs from ref: White means the base is different from the reference, dark grey means the base matches the reference.

To reshape the input example to be compatible with the Med-PaLM M input shape of (224, 224, 3), MultiMedBench uses stacked up channels 1, 2, 3 with channels 4, 5, 6 such that the original tensor of shape (100, 221, 6) became an RGB image of shape (200, 221, 3). MultiMedBench then padded the image on the width and height dimensions to give it a final shape of (224, 224, 3).

VQA-RAD is a radiology visual question answering (VQA) dataset which consists of 315 radiology images and 3,515 question-answer pairs created and validated by clinicians. The radiology images are selected from three imaging modalities (CT, MRI, and X-rays) and three anatomical regions (head, abdominal, chest). The types of question fall into 11 categories including modality, plane, organ system, abnormality, size, plane, positional reasoning, color, counting, attribute and other. 58% of the question-answer (QA) pairs are closed-ended (yes/no or limited choices) and the rest 42% are open-ended (short answer). MultiMedBench adopts the official train/test splits, where the training set contains 1,797 QA pairs (only free-form and paraphrased questions were included) and the test set contains 451 QA pairs (not filtered).

Path-VQA is a pathology VQA dataset, containing a total of 4,998 pathology images with 32,799 question-answer pairs. Pathology images are extracted from medical textbooks and online digital libraries. Each image is associated with multiple QA pairs pertaining to different aspects of the pathology including color, location, appearance, shape, etc. Open-ended questions account for 50.2% of all questions, which are categorized into 7 categories: what, where, when, whose, how, and how much/how many, accounting for 50.2% of all questions. The rest are close-ended questions with simple “yes/no” answer. MultiMedBench adopts the official data partitioning where the training, validation, and test sets contain 19,755, 6,279, and 6,761 QA pairs, respectively.

Slake-VQA is a semantically annotated and knowledge-enhanced bilingual (English and Chinese) VQA dataset on radiology images. It contains 642 annotated images with 14,028 question-answer pairs covering 12 diseases, 39 organ systems and 3 imaging modalities (CT, MRI, and chest X-rays). Questions are either open-ended (free-form) or closed-ended (balanced yes/no) related to various aspects of the image content including plane, quality, position, organ, abnormality, size, color, shape, knowledge graph, etc. The training, validation, and test sets contain 9,849, 2,109, and 2,070 samples, respectively.

MIMIC-CXR is a large dataset of chest radiographs with free-text radiology reports. A total of 377,110 images are available in the dataset from 227,835 image studies collected for 65,379 patients. Each patient may have multiple studies and each study may contain one or more images associated with the same free-text report. Images in MIMIC-CXR are collected from multiple view positions: e.g., anterior-posterior (AP), posterioranterior, and lateral (LA). Protected health information (PHI) in radiology reports and images is removed, which results in missing information in some sentences of the reports. Since this dataset contains sequential imaging studies of an individual patient, a large number of reports refer to information in prior studies of the same patient. Each report is annotated with structured labels of 14 common radiological observations using CheXpert labeler.

MultiMedBench includes two tasks using this dataset: chest X-ray report generation and binary classification of clinically-relevant pathology observations. The system preprocessed the radiology reports by extracting the indication, findings, and impression sections, removing redundant white-spaces in the reports, following previous work.

MultiMedBench adopts the official train/validation/test splits. Images without reports are discarded, and reports where the findings section can not be extracted across train and test are discarded. Filtering was applied to remove the reports where the length of findings section exceeds 800 characters. However, unlike most previous work using focusing only on the frontal view, images of different orientation that are associated with the same report are treated as independent samples (retaining the patient-level train/test splits to avoid contamination of the test data). The goal is to improve the image understanding capability of the model to process images of different view positions. In a separate evaluation, a subset of samples where reports are accompanied by both a front and lateral view (two-view report generation) are provided for additional evaluation.

For the report generation task, MultiMedBench combines the chest X-ray image with the contextual information from the indication section (reason for the study) to predict the findings section of the target report. The total number of samples in the training, validation, and test sets are: 353,542, 2,866, and 4,834, respectively. For the binary classification task, MultiMedBench groups negative and uncertain labels as the negative class for 11 pathological conditions: no finding, atelectasis, cardiomegaly, consolidation, edema, pleural effusion, lung opacity, enlarged cardiomediastinum, fracture, pneumonia, and support devices. Atelectasis, cardiomegaly, consolidation, edema, and pleural effusion are 5 major conditions given their clinical relevance and prevalence. The “No finding” label captures the cases without any pathology and therefore this classification task simply helps the model to distinguish normal cases from cases with any type of abnormality. Due to class imbalance, during training upsampling was applied to the positive class by a factor of 2 for the following conditions: consolidation, enlarged cardiomediastinum, fracture, and pneumonia. These binary classification tasks are auxiliary to the report generation task when they are trained simultaneously since they help the model to distinguish among different types of clinical observations in the chest X-ray images.

For the present results, all the images in MultiMedBench were resized to 224 by 224, while preserving the original aspect ratio with padding if needed. The gray-scale images were converted to 3-channel images by stacking up the same image along the channel dimension. Task-specific prepossessing methods such as class balancing and image data augmentation are described in detail for each task in Tu et al., Towards Generalist Biomedical AI, arXiv: 2307.14334v1 (Jul. 26, 2023), which is incorporated by reference herein in its entirety.

For the present example results, a model training system trained the model with a mixture of distinct tasks simultaneously via instruction tuning. Specifically, the training system provided the model with task-specific instructions to prompt the model to perform different types of tasks in a unified generative framework. The task prompt consisted of an instruction, relevant context information, and a question. Examples are shown in the preceding figures. The training system formulated all classification tasks as multiple choice questions where all possible class labels are provided as individual answer options and the model was prompted to generate the most likely answer as the target output. For other generative tasks such as visual question answering and report generation and summarization, the model was finetuned on the target response.

For the majority of tasks, the training system added a text-only one-shot exemplar to the task prompt to condition the language model's prediction.

For multimodal tasks, the training system replaced the actual image in the exemplar with a dummy text placeholder (with the text string “<img>”): this (i) preserves training compute efficiency for single-image training, and also (ii) bypasses potential interference from cross-attention between a given text token and image tokens from multiple images.

The training system finetuned the pretrained 12B, 84B, and 562B parameter variants of PaLM-E on MultiMedBench tasks with mixture ratios denoted in Table 1. These mixture ratios were empirically determined such that they are approximately proportional to the number of training samples in each dataset and ensuring at least one sample from each task is present in one batch. The training system performed an end-to-end finetuning of the PaLM-E model with the entire set of model parameters updated during training. For multimodal tasks, image tokens were interleaved with text tokens to form multimodal context input to the PaLM-E model.

In the present example, the multimodal context input contained at most 1 image for all finetuning tasks. However, Med-PaLM M is able to process inputs with multiple images during inference.

The training system used the Adafactor optimizer with momentum of 0.9, dropout rate of 0.1, and a constant learning rate schedule.

The training system used different sets of hyperparameters in our finetuning experiments for different model sizes, which are further detailed in Table 4.

TABLE 4

| | Med-PaLM M | Med-PaLM M | Med-PaLM M |
|---|---|---|---|
| Hyperparameter | (12B) | (84B) | (562B) |
| Learning rate | 5 × 10⁻⁵ | 5 × 10⁻⁵ | 2.5 × 10⁻⁵ |
| Batch size | 128 | 128 | 256 |
| Max token input | 710 | 710 | 710 |
| length | | | |
| Max token output | 256 | 256 | 256 |
| length | | | |

The resulting model, Med-PaLM M (12B, 84B, and 562B), is adapted to the biomedical domain with the capability to encode and interpret multimodal inputs and perform tasks including medical (visual) question answering, radiology report generation and summarization, medical image classification, and genomic variant calling.

Med-PaLM M was simultaneously finetuned on a mixture of language-only and multimodal biomedical tasks in MultiMedBench. The example results assess the model's in-distribution performance on these tasks by comparing to the corresponding SOTA results obtained from separate specialist models. Specifically, the training system used the same few-shot setup as in training for each task during evaluation. Task-specific metrics were computed on the test split of each task and compared to prior SOTA specialist AI systems.

MultiMedBench test results are reported below for an example implementation of the present disclosure, referred to herein as “Med-PaLM M.” Results are summarized in Table 5.

TABLE 5

| | | | | | PaLM-E | Med-PaLM M |
|---|---|---|---|---|---|---|
| Task Type | Modality | Dataset | Metric | SOTA | (84B) | (Best) |
| Question | Text | MedQA | Accuracy | 86.50% [61] | 28.83% | 69.68% |
| Answering | | MedMCQA | Accuracy | 72.30% [61] | 33.35% | 62.59% |
| | | PubMedQA | Accuracy | 81.80% [61] | 64.00% | 80.00% |
| Report | Radiology | MIMIC-H1 | ROUGE-L | 88.70% [62] | 3.30% | 32.03% |
| Summarization | | | BLEU | 18.20% [62] | 0.34% | 15.36% |
| | | | F1-RadGraph | 40.80% [62] | 8.00% | 34.71% |
| Visual | Radiology | VQA-RAD | BLEU-1 | 71.03% [63] | 59.19% | 71.27% |
| Question | | | F1 | N/A | 38.67% | 62.06% |
| Answering | | Slake-VQA | BLEU-1 | 78.60% [64] | 52.65% | 92.7% |
| | | | F1 | 78.10% [64] | 24.53% | 89.28% |
| | Pathology | Path-VQA | BLEU-1 | 70.30% [64] | 54.92% | 72.27% |
| | | | F1 | 58.40% [64] | 29.68% | 62.69% |
| Report | Chest X-ray | MIMIC-CXR | Micro-F1-14 | 44.20% [65] | 18.40% | 53.56% |
| Generation | | | Macro-F1-14 | 30.70% [65] | 10.11% | 39.83% |
| | | | Micro-F1-5 | 56.70% [65] | 5.51% | 57.88% |
| | | | Macro F1-5 | N/A | 4.85% | 51.60% |
| | | | F1-RadGraph | 24.40% [14] | 11.66% | 26.71% |
| | | | BLEU-1 | 39.48% [65] | 19.86% | 32.31% |
| | | | BLEU-4 | 13.30% [66] | 4.60% | 11.50% |
| | | | ROUGE-L | 29.60% [67] | 16.53% | 27.49% |
| | | | CIDEr-D | 49.50% [68] | 3.50% | 26.17% |
| Image | Clust X-ray | MIMI-CXR | Macro-AUC | 81.27% [69] | 51.48% | 79.09% |
| Classification | | (5 conditions) | Macro-F1 | N/A | 7.83% | 41.57% |
| | Dermatology | PAD-UFES-20 | Macro-AUC | N/A | 63.37% | 97.27% |
| | | | Macro-F1 | N/A | 1.38% | 84.32% |
| | Mammography | VinDr-Mammo | Macro-AUC | 64.50% [49] | 51.49% | 71.78% |
| | | | Macro-F1 | N/A | 16.06% | 35.70% |
| | | CBIS-DDSM | Macro-AUC | N/A | 47.75% | 73.31% |
| | | (mass) | Macro-F1 | N/A | 7.77% | 51.12% |
| | | CBIS-DDSM | Macro-AUC | N/A | 40.67% | 82.22% |
| | | (calcification) | Macro-F1 | 70.71% [70] | 11.37% | 67.86% |
| | Genomics | PrecisionFDA | Indel-F1 | 99.40% [71] | 53.01% | 97.04% |
| | (Variant Calling) | (Truth Challenge V2) | SNP-F1 | 99.70% [71] | 52.84% | 99.35% |

Across MultiMedBench tasks, Med-PaLM M's best result (across three model sizes) exceeded prior SOTA results on 5 out of 12 tasks while being competitive on the rest. Notably, these results were achieved with a generalist model using the same set of model weights without any task-specific architecture customization or optimization.

On medical question answering tasks, the training system compared against the SOTA Med-PaLM 2 result and observed higher performance of Med-PaLM 2. However, when compared to the baseline PaLM model on which Med-PaLM M was built, Med-PaLM M outperformed the previous best PaLM result by a large margin in the same few-shot setting on all three question answering datasets.

Further, when compared to PaLM-E 84B as a generalist baseline without biomedical domain finetuning, Med-PaLM M exhibited performance improvements on all 14 tasks often by a significant margin, demonstrating the importance of domain adaptation. Taken together, these results illustrate the strong capabilities of Med-PaLM M as a generalist biomedical AI model.

Few-shot performance is reported in Table 6.

TABLE 6

| Dataset | Med-PaLM 2 | PaLM | Med-PaLM M (12B) | Med-PaLM M (84B) | Med-PaLM M (562B) |
|---|---|---|---|---|---|
| MedQA (USMLE) | 86.50% | 58.90% | 29.22% | 46.11% | 69.68% |
| MedMCQA | 72.30% | 54.50% | 32.20% | 47.60% | 62.59% |
| PubMedQA | 81.80% | 55.00% | 48.60% | 71.40% | 80.00% |

Med-PaLM M performance on MIMIC-III radiology report summarization tasks is reported in Table 7.

TABLE 7

| Dataset | Metric | SOTA | Med-PaLM M (12B) | Med-PaLM M (84B) | Med-PaLM M (562B) |
|---|---|---|---|---|---|
| MIMIC-III | ROUGE-L | 38.70% | 29.45% | 31.47% | 32.03% |
| | BLEU | 16.20% | 12.14% | 15.36% | 15.21% |
| | F1-RadGraph | 40.80% | 31.43% | 33.96% | 34.71% |

Med-PaLM M performance on medical image classification tasks is reported in Table 8.

TABLE 8

| | | | | Med-PaLM M | Med-PaLM M | Med-PaLM M |
|---|---|---|---|---|---|---|
| Dataset | # Classes | Metric | SOTA | (12B) | (84B) | (562B) |
| MIMIC-CXR | 2-class | Macro-AUC | 81.27% | 76.67% | 78.35% | 70.09% |
| (5 conditions) | | Macro-F1 | N/A | 38.33% | 36.83% | 41.57% |
| PAD-UFES-20 | 6-class | Macro-AUC | N/A | 95.57% | 97.27% | 96.08% |
| | | Macro-F1 | N/A | 78.42% | 84.32% | 77.03% |
| Variant Calling | 3-class | Indel- F1 | 99.40% | 96.42% | 97.04% | 96.46% |
| | | SNP-F1 | 89.70% | 99.35% | 99.32% | 90.16% |
| VinDr-Mammo | 5-class | Macro-AUC | 64.50% | 66.29% | 71.76% | 71.42% |
| | | Macro-F1 | N/A | 29.81% | 35.7% | 33.90% |
| CBIS-DDSM | 3-class | Macro-AUC | N/A | 70.11% | 73.09% | 73.31% |
| (mass) | | Macro-F1 | N/A | 47.23% | 49.98% | 51.12% |
| CBIS-DDSM | 3-class | Macro-AUC | N/A | 81.40% | 82.22% | 80.90% |
| (calcification) | | Macro-F1 | 70.71% | 67.86% | 63.81% | 63.08% |

The classification tasks cover multiple domains including dermatology, radiology, and genomics. Since these tasks all have imbalanced class distributions, the table reports macro-AUC (unweighted mean of all the per-class AUC scores) and macro-F1 scores (unweighted mean of all the per-class F1 scores) as the classification metrics except for the genomic variant calling task where the F1 scores for single nucleotide polymorphisms (SNPs) and short insertions and deletions (indels) in the context of variant discovery were used instead. On VinDr-Mammo, all size variants of Med-PaLM M exceeded the prior SOTA using a smaller ViT (9.7M) on macro-AUC. On CBIS-DDSM, Med-PaLM M achieved the best macro-F1 of 51.12% and 67.86% on the mass and calcification classification, respectively, behind the SOTA F1 of 70.71% reported on the calcification classification. Note that most previous works on CBIS-DDSM focused on a two-class patch-level classification (benign versus malignant) problem in contrast to this 3-class setup. On Pad-UFES-20, since no official train/test splits are available, the results are not directly comparable with prior studies. Med-PaLM M 84B achieved a macro-AUC of 97.27%, on par with previous reported results (94%-98%) obtained using CNN and ViT variants. On MIMIC-CXR, the table reports the macro-average of F1 scores across the binary classification of 5 major conditions: atelectasis, cardiomegaly, consolidation, edema, and pleural effusion. Med-PaLM M (562B) achieved a macro-AUC of 79.09%, slightly lower than the SOTA result of 81.27% obtained from ParallelXNet, which used a parallelization of various CNN Architectures. On the variant calling task, Deep Variant model outperformed Med-PaLM M on both Indel-F1 and SNP-F1 scores. The SOTA Deep Variant model was trained with 2,633-fold more training examples. Training with the same examples resulted in a narrower advantage for Deep Variant for SNP (Med-PaLM M 99.35% versus Deep Variant 99.63%) and Indel (Med-PaLM M 97.04% versus Deep Variant 98.55%. Notably, Med-PaLM M outperformed the accuracy of the widely used GATK4 method for SNP calling (Med-PaLM M 99.35% versus GATK4 99.29%) but not Indel calling (Med-PaLM M 97.04% versus GATK4 99.32%).

Taken together, Med-PaLM M achieved competitive results on a variety of classification tasks using a single model compared to highly specialized SOTA models. It is worth noting that Med-PaLM M did not benefit from any fine-grained task-specific customization and hyperparameter tuning beyond data augmentation and class balancing.

Med-PaLM M performance on medical visual question answering tasks is reported in Table 9.

TABLE 9

| Dataset | Metric | SOTA | Med-PaLM M (12B) | Med-PaLM M (84B) | Med-PaLM M (562B) |
|---|---|---|---|---|---|
| VQA-RAD | BLEU-1 | 71.03% | 64.02% | 69.38% | 71.27% |
| | F1 | N/A | 50.66% | 59.90% | 62.06% |
| Path-VQA | BLEU-1 | 70.30% | 68.97% | 70.16% | 72.27% |
| | F1 | 58.40% | 57.24% | 59.51% | 62.69% |
| Slake VQA | BLEU-1 | 78.60% | 90.77% | 92.7% | 91.64% |
| | F1 | 78.10% | 86.22% | 89.28% | 87.50% |

To probe Med-PaLM M's ability to generalize to previously unseen medical concepts, the training system evaluated the model's ability to predict the presence or absence of tuberculosis (TB) from chest X-ray images. The training system used the Montgomery County chest X-ray set (MC) for this purpose. The dataset contains 138 frontal chest X-rays, of which 80 are normal cases and 58 cases have manifestations of TB. Each case also contains annotations on the abnormality seen in the lung. Med-PaLM M was been trained on MIMIC-CXR dataset, but it was not trained to explicitly predict the TB disease label.

The training system evaluated the accuracy across model scales by formulating this problem as a two-choice question answering task where the model was prompted (with a text-only one-shot exemplar) to generate a yes/no answer about the presence of TB in the input image. Table 10 reports the results.

TABLE 10

| Model | # Training samples | Accuracy |
|---|---|---|
| SOTA [74] | 138 | 92.60% |
| Med-PaLM M (12B) | 0 | 86.96% |
| Med-PaLM M (84B) | 0 | 82.60% |
| Med-PaLM M (562B) | 0 | 87.68% |

The training system further explored zero-shot chain-of-thought (CoT) multimodal medical reasoning ability of the model by prompting with a text-only exemplar (without the corresponding image) and prompting the model to generate the class prediction and an accompanying report describing the image findings. While the training system did prompt the model with a single text-only input-output pair, the training system omitted the image (used a dummy text placeholder instead) and the text exemplar was hand-crafted rather than drawn from the training set. Hence, this approach can be considered zero-shot rather than one-shot.

In order to assess Med-PaLM M's ability to generalize to novel task scenarios, the training system evaluated the model performance on two-view chest X-ray report generation—this is a novel task given the model was trained to generate reports only from a single-view chest X-ray. Med-PaLM M performance remains competitive on a novel two-view report generation task setup despite having not been trained with two visual inputs before. Med-PaLM M achieves SOTA results on clinical efficacy metrics for the two view report generation task. Table 11 reports the performance for various model configurations.

TABLE 11

| | | Med-PaLM | Med-PaLM | Med-PaLM |
|---|---|---|---|---|
| Metric | SOTA | M (12B) | M (84B) | M (562B) |
| Micro-F1-14 | 44.20% | 51.41% | 53.56% | 51.60% |
| Macro-F1-14 | 30.70% | 37.31% | 39.83% | 37.81% |
| Micro-F1-5 | 56.70% | 56.54% | 57.88% | 56.28% |
| Macro-F1-5 | N/A | 50.57% | 51.60% | 49.86% |
| F1-RadGraph | 24.40% | 25.20% | 26.71% | 26.06% |
| BLEU-1 | 39.48% | 30.90% | 32.31% | 31.73% |
| BLEU-1 | 13.30% | 10.43% | 11.31% | 11.50% |
| ROUGE-L | 29.60% | 26.16% | 27.29% | 27.49% |
| CIDEr-D | 49.50% | 23.43% | 26.17% | 25.27% |

The training system also probed for evidence of positive task transfer as a result of jointly training a single generalist model to solve many different biomedical tasks. To this end, the training system performed an ablation study where the training system trained a Med-PaLM M 84B variant by excluding the MIMIC-CXR classification tasks from the task mixture. The training system compared this model variant to the Med-PaLM M 84B variant trained on the complete MultiMedBench mixture on the chest X-ray report generation task with the expectation of improved performance in the latter. Table 12 reports the performance.

TABLE 12

| | | Med-PaLM M | Med-PaLM M (84B) |
|---|---|---|---|
| Dataset | Metric | (84B) | No CXR classification |
| MIMIC-CXR | Micro-F1-14 | 53.56% | 52.94% |
| | Macro-F1-14 | 39.83% | 38.92% |
| | Micro-F1-5 | 57.88% | 57.58% |
| | Macro-F1-5 | 51.60% | 51.32% |
| | F1-RadGraph | 26.71% | 26.08% |
| | BLEU-1 | 32.31% | 31.72% |
| | BLEU-4 | 11.31% | 10.87% |
| | ROUGE-L | 27.29% | 26.67% |
| | CIDEr-D | 26.17% | 25.17% |
| MIMIC-CXR | Macro-AUC | 78.35% | 73.88% |
| (5 conditions | Macro-F1 | 36.83% | 43.97% |

To demonstrate the positive task transfer arising from joint training across modalities and tasks, the training system performed an ablation study where the training system trained a Med-PaLM M 84B variant by excluding the MIMIC-CXR classification task from the task mixture and compared this model variant against Med-PaLM M 84B trained on the full MultiMedBench mixture.

The training system observed that the model trained jointly on both report generation and classification has higher performance across the board on all report generation metrics.

The training system also generated results indicating that the model trained only on chest X-ray report generation can generalize to abnormality detection in a zero-shot fashion with compelling performance, as evidenced by a higher macro-F1 score. This is another example of generalization to a novel task setting where the model learns to differentiate between types of abnormalities from training on the more complex report generation task.

To further assess the quality and clinical applicability of chest X-ray reports generated by Med-PaLM M and understand the effect of model scaling, the training system conducted a human evaluation using the MIMIC-CXR dataset. The evaluation was performed by four qualified thoracic radiologists based in India.

The evaluation set consisted of 246 cases selected from the MIMIC-CXR test split. To match the expected input format of Med-PaLM M, the training system selected a single image from each study. The training system excluded studies that had ground truth reports mentioning multiple X-ray views or past examinations of the same patient.

The training system conducted two complementary human evaluations: (1) a side-by-side evaluation in which raters compared multiple alternative report findings and ranked them based on their overall quality, and (2) an independent evaluation, where raters assessed the quality of individual report findings.

Prior to performing the final evaluation, the training system iterated upon the instructions for the raters and calibrated their grades using a pilot set of 25 cases that were distinct from the evaluation set. Side-by-side evaluation was performed for all 246 cases, where each case was rated by a single radiologist randomly selected from a pool of four. For independent evaluation, each of the four radiologists independently annotated findings generated by three Med-PaLM M model variants (12B, 84B, and 562B) for every case in the evaluation set. Radiologists were blind to the source of the report findings for all evaluation tasks, and the reports were presented in a randomized order.

The input to each side-by-side evaluation was a single chest X-ray, along with the “indication” section from the MIMIC-CXR study. Four alternative options for the “findings” section of the report were shown to raters. The four alternative “findings” sections corresponded to the dataset reference report's findings, and findings generated by three Med-PaLM M model variants (12B, 84B, 562B). Raters were asked to rank the four alternative findings based on their overall quality using their best clinical judgement.

Averaged over all four raters, the radiologist-provided reference report was ranked best in 37.14% of cases, followed by Med-PaLM M (84B) which was ranked best in 25.78% of cases, and the other two model scales, 12B and 562B, which were ranked best in 19.49% and 17.59% of cases respectively. To enable a direct comparison of reports generated by each Med-PaLM M model scale to the radiologist-provided reference report, the training system facilitated pairwise preferences from the four-way ranking and provided a breakdown for each rater and model scale. Averaged over all four raters, Med-PaLM M 84B was preferred over the reference report in 40.50% of cases, followed by the other two model scales, 12B and 562B, which were preferred over the reference report in 34.05% and 32.00% of cases, respectively.

For independent evaluation, raters were also presented with a single chest X-ray, along with the indication and reference report's findings from the MIMIC-CXR study (marked explicitly as such), but this time only a single findings paragraph generated by Med-PaLM M. Raters were asked to assess the quality of the Med-PaLM M generated findings in the presence of the reference inputs provided and their own judgement of the chest X-ray image.

First, raters assessed whether the quality and view of the provided image were sufficient to perform the evaluation task fully.

Next, they annotated all passages in the model-generated findings that they disagreed with (errors), and all missing parts (omissions).

Raters categorized each error passage by its type (no finding, incorrect finding location, incorrect severity, reference to non-existent view or prior study), assessed its clinical significance, and suggested alternative text to replace the selected passage. Likewise, for each omission, raters specified a passage that should have been included and determined if the omission had any clinical significance.

For omissions, the training system reported the lowest rate of 0.12 (95% CI, 0.10-0.15) omissions per report on average for both the Med-PaLM M 12B and 84B models, followed by 0.13 (95% CI, 0.11-0.16) for the 562B model. The training system measured the lowest mean error rate of 0.25 (95% CI, 0.22-0.28) for Med-PaLM M 84B, followed by 0.28 (95% CI, 0.24-0.31) for Med-PaLM M 12B and 0.29 (95% CI, 0.25-0.32) for the 562B model. Notably, this error rate is comparable to those reported for human radiologists baselines on the MIMIC-CXR dataset.

For the purpose of analysis, the training system provided a feedback interface that distinguished between clinical errors (i.e., “Finding I do not agree is present”, “Incorrect location of finding”) and non-clinical errors (i.e., “Refers to view that is not present” or “Refers to study that is not present”).

### Example Methods

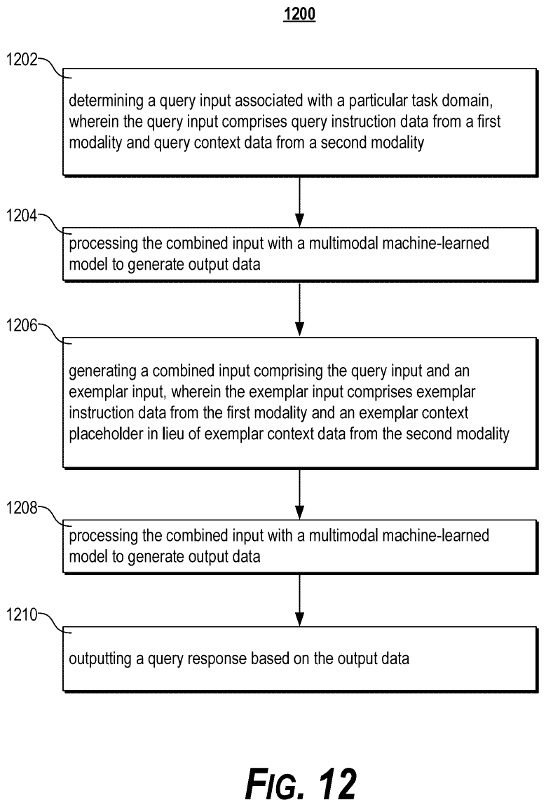

FIG. 12 depicts a flowchart of a method 1200 for controlling one or more machine-learned models according to aspects of the present disclosure. For instance, an example machine-learned model can include a machine-learned model 108.

One or more portion(s) of example method 1200 can be implemented by a computing system that includes one or more computing devices such as, for example, computing systems described with reference to the other figures. Each respective portion of example method 1200 can be performed by any (or any combination) of one or more computing devices. Moreover, one or more portion(s) of example method 1200 can be implemented on the hardware components of the device(s) described herein, for example, to train one or more systems or models. FIG. 12 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, or modified in various ways without deviating from the scope of the present disclosure. FIG. 12 is described with reference to elements/terms described with respect to other systems and figures for exemplary illustrated purposes and is not meant to be limiting. One or more portions of example method 1200 can be performed additionally, or alternatively, by other systems.

At 1202, example method 1200 can include receiving a query associated with a particular task domain. A query can be a query 210. Query 210 can be associated with a task domain. Example task domains can include question answering, report generation and summarization, visual question answering, medical image classification, genomic variant calling, etc. Example subject matter domains can include general medical subject matter, radiology, pathology, dermatology, mammography, genomics, etc.

At 1204, example method 1200 can include generating, based on the query, a query input. Generating the query input can include embedding data from the query (e.g., projecting data elements from the query into a latent space, such as by generating a vector embedding characterized by the dimensions of the latent space). The query input can be, for example query input 104-2. The query input can include query instruction data from a first modality and query context data from a second modality. For example, query instruction data can include query 224 that instructions or asks the model to perform a task or answer a question. Query context data can include query context 226 that provides additional information to help the system response to the query. Query context data can be in a different modality from query instruction data. For instance, query instruction data can be in a text or audio modality (e.g., corresponding to a natural language instruction). Query context data can be in an image, video, audio, text, waveform, sensor time history, etc. modality. Example multimodal query inputs are described with respect to FIGS. 3 to 5.

At 1206, example method 1200 can include generating a combined input that can include the query input and an exemplar input. For example, the combined input can be a combined input 104. An example exemplar input can be exemplar input 104-1. In the combined input, the exemplar input can include exemplar instruction data from the first modality (e.g., exemplar query 216) and an exemplar context placeholder (e.g., context placeholder 218) in lieu of exemplar context data from the second modality. For instance, substitution operator 220 can substitute a portion of exemplar 202 for a placeholder value. For instance, instead of image data, substitution operator 220 can insert a placeholder sequence in its place. Exemplar input 104-1 can include exemplar response 222 that represents first modality data 208. In this manner, for instance, exemplar input 104-1 can provide an illustrative example of how an example query can be answered, even without including the context from other modalities.

At 1206, example method 1200 can include processing the combined input with a multimodal machine-learned model to generate output data. For example, a machine-learned model 108 can process combined input 104 to generate output data 110.

At 1208, example method 1200 can include outputting a query response based on the output data. For instance, a query response can be an answer to a question or a response to an instruction to perform a task (e.g., a classification output, a diagnostic output, a recognition output, a summary output, etc.).

In some implementations of example method 1200, the machine-learned model is a sequence processing model. For example, machine-learned model 108 can be a sequence processing model that processes sequences of input values. The sequence of input values can include input values representing multiple different modalities of data. The input values representing multiple different modalities of data can be interleaved together. In some implementations of example method 1200, the machine-learned model can include one or more transformer layers.

In some implementations of example method 1200, the query input can include the instruction data from the first modality interleaved with the context data from the second modality. For instance, tokens representing image data (e.g., a second modality) can be interleaved with tokens representing natural language data (e.g., a first modality).

In some implementations of example method 1200, the machine-learned model can include one or more first modality input layers configured to process data from the first modality and project the data from the first modality into a latent space of the machine-learned model. For example, one or more layers of an input encoder portion of the model can receive first modality data of a query 210 and embed the first modality data into a latent representation.

In some implementations of example method 1200, the machine-learned model can include one or more second modality input layers configured to process data from the second modality and project the data from the second modality into the latent space. For example, one or more layers of an input encoder portion of the model can receive second modality data of a query 210 and embed the second modality data into a latent representation.

In some implementations, example method 1200 can include detecting second modality data in the query. For instance, a system can parse query 210 to identify what type(s) of data are contained in query 210. Data of each modality can be directed to input encoders that are configured for processing the respective modality. For instance, in some implementations, example method 1200 can include, responsive to detecting second modality data in the query, routing the second modality data to a machine-learned sequence encoder configured to process the second modality data and generate a sequence representing the second modality data. In some implementations, example method 1200 can include, based on detecting the second modality data in the query, selecting the machine-learned sequence encoder from among a plurality of modality-specific machine-learned sequence encoders.

In some implementations, example method 1200 can include generating a score based on the output data. A score can be a loss evaluated using a string reconstruction metric. A score can be generated using a learned evaluation that compares a latent representation of a test output against a latent representation of a reference output. Example learned metrics can include BLUERT, etc. In some implementations, example method 1200 can include training the multimodal machine-learned model based on the score. For instance, a gradient can be computed for one or more parameters of the model that indicate how a change in the parameter would effect the score value. The one or more parameters can be updated in a direction that is expected to improve the score.

Example training iterations can be performed in batches. In some implementations, example method 1200 can include processing a training batch with the multimodal machine-learned model to generate output data. Batch processing of inputs is described with various examples shown in FIG. 9. Training data 900 can represent a diverse array of data. Training batch inputs 902 can be sampled from training data 900. Training batch inputs 902 can include example inputs from various different domains. Training batch inputs 902 can be processed by machine-learned model 108 to generate training batch outputs 904.

In some implementations of example method 1200, the training batch can include a plurality of training query inputs that can include, for each respective task domain of a plurality of task domains, a respective set of training query inputs associated with the respective task domain, each training query input in the respective set of training query inputs that can include instruction data in a first modality and context data in a second modality (e.g., a multimodal training query input).

In some implementations, example method 1200 can include outputting, based on the output data, training query responses respectively corresponding to the plurality of training query inputs.

In some implementations, example method 1200 can include training the multimodal machine-learned model based on evaluations of the training query responses.

In some implementations of example method 1200, the training batch can include a unimodal set of training query inputs associated with a unimodal task domain. For instance, the training batch can also include single-modality queries (e.g., text only). Each training query input in the unimodal set of training query inputs can include instruction data in a first modality and context data in the first modality.

In some implementations of example method 1200, the training batch can include at least four query inputs associated with each respective task domain.

In some implementations of example method 1200, the training batch can include at least one query input associated with two or more of the following task domains: question answering; report summarization; visual question answering; report generation; and image classification.

In some implementations of example method 1200, the training batch can include at least one query input associated with each of the following task domains: question answering; report summarization; visual question answering; report generation; and image classification.

In some implementations of example method 1200, the training batch can include, for the visual question answering task domain: at least one query input associated with a radiology task; and at least one query input associated with a pathology task.

In some implementations of example method 1200, over half the training batch is associated with the report generation task.

In some implementations of example method 1200, the training batch can include a plurality of exemplar inputs respectively associated with the plurality of query inputs, wherein at least one of the plurality of exemplar inputs is unimodal.

In some implementations, example method 1200 can include detecting second modality data in a training query. In some implementations, example method 1200 can include selecting, from among a plurality of modality-specific machine-learned sequence encoders, a machine-learned sequence encoder configured to process the second modality data and generate a sequence representing the second modality data. In some implementations, example method 1200 can include routing the second modality data to the machine-learned sequence encoder for processing.

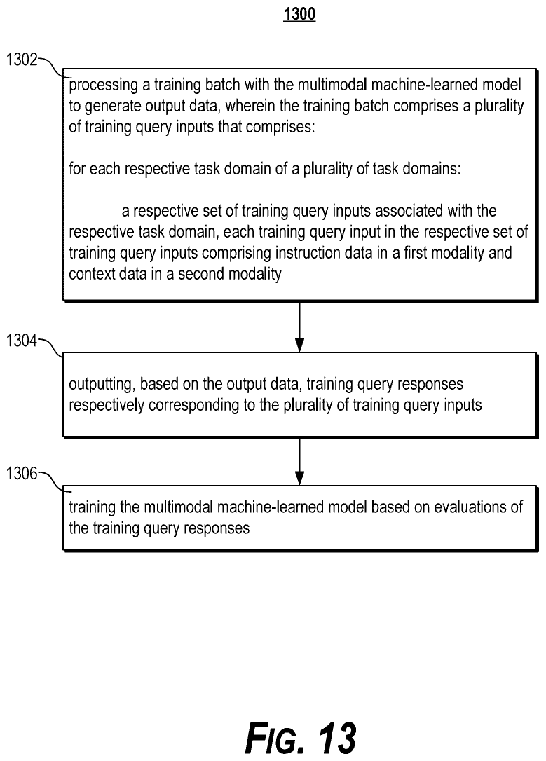

FIG. 13 depicts a flowchart of a method 1300 for training one or more machine-learned models according to aspects of the present disclosure. For instance, an example machine-learned model can include a machine-learned model 108.

One or more portion(s) of example method 1300 can be implemented by a computing system that includes one or more computing devices such as, for example, computing systems described with reference to the other figures. Each respective portion of example method 1300 can be performed by any (or any combination) of one or more computing devices. Moreover, one or more portion(s) of example method 1300 can be implemented on the hardware components of the device(s) described herein, for example, to train one or more systems or models. FIG. 13 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, or modified in various ways without deviating from the scope of the present disclosure. FIG. 13 is described with reference to elements/terms described with respect to other systems and figures for exemplary illustrated purposes and is not meant to be limiting. One or more portions of example method 1300 can be performed additionally, or alternatively, by other systems.

At 1302, example method 1300 can include processing a training batch with a multimodal machine-learned model to generate output data. An example multimodal machine-learned model is machine-learned model 108. Example training iterations can be performed in batches. In some implementations, example method 1200 can include processing a training batch with the multimodal machine-learned model to generate output data. Batch processing of inputs is described with various examples shown in FIG. 9. Training data 900 can represent a diverse array of data. Training batch inputs 902 can be sampled from training data 900. Training batch inputs 902 can include example inputs from various different domains. Training batch inputs 902 can be processed by machine-learned model 108 to generate training batch outputs 904.

In some implementations of example method 1300, the training batch can include a plurality of training query inputs. The plurality of training query inputs can include, for each respective task domain of a plurality of task domains, a respective set of training query inputs associated with the respective task domain. Each training query input in the respective set of training query inputs can include instruction data in a first modality and context data in a second modality (e.g., a multimodal training query input).

At 1304, example method 1300 can include outputting, based on the output data, training query responses respectively corresponding to the plurality of training query inputs. Machine-learned model 108 can process at least a portion of the training batch in parallel. For instance, an input to the model can have a batch dimension, with the members of a training batch populating the input data structure along the batch dimension.

At 1306, example method 1300 can include training the multimodal machine-learned model based on evaluations of the training query responses. In some implementations, example method 1300 can include generating a score based on the output data. A score can be a loss evaluated using a string reconstruction metric. A score can be generated using a learned evaluation that compares a latent representation of a test output against a latent representation of a reference output. Example learned metrics can include BLUERT, etc. In some implementations, example method 1300 can include training the multimodal machine-learned model based on the score. For instance, a gradient can be computed for one or more parameters of the model that indicate how a change in the parameter would effect the score value. The one or more parameters can be updated in a direction that is expected to improve the score.

In some implementations of example method 1300, the training batch can include a unimodal set of training query inputs associated with a unimodal task domain, each training query input in the unimodal set of training query inputs that can include instruction data in a first modality and context data in the first modality.

In some implementations of example method 1300, the training batch can include at least four query inputs associated with each respective task domain.

In some implementations of example method 1300, the training batch can include at least one query input associated with two or more of the following task domains: question answering; report summarization; visual question answering; report generation; and image classification.

In some implementations of example method 1300, the training batch can include at least one query input associated with each of the following task domains: question answering; report summarization; visual question answering; report generation; and image classification.

In some implementations of example method 1300, the training batch can include, for the visual question answering task domain: at least one query input associated with a radiology task; and at least one query input associated with a pathology task.

In some implementations of example method 1300, over half the training batch is associated with the report generation task.

In some implementations of example method 1300, the training batch can include a plurality of exemplar inputs respectively associated with the plurality of query inputs, wherein at least one of the plurality of exemplar inputs is unimodal.

In some implementations, example method 1300 can include detecting second modality data in a training query.

In some implementations, example method 1300 can include selecting, from among a plurality of modality-specific machine-learned sequence encoders, a machine-learned sequence encoder configured to process the second modality data and generate a sequence representing the second modality data.

In some implementations, example method 1300 can include routing the second modality data to the machine-learned sequence encoder for processing.

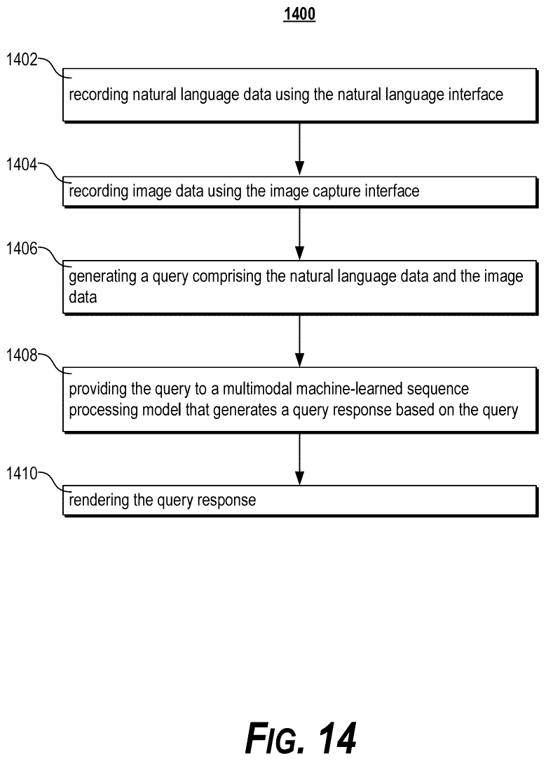

FIG. 14 depicts a flowchart of a method 1400 for implementing one or more machine-learned models according to aspects of the present disclosure. For instance, an example machine-learned model can include a machine-learned model 108.

One or more portion(s) of example method 1400 can be implemented by a computing system that includes one or more computing devices such as, for example, computing systems described with reference to the other figures. Each respective portion of example method 1400 can be performed by any (or any combination) of one or more computing devices. Moreover, one or more portion(s) of example method 1400 can be implemented on the hardware components of the device(s) described herein, for example, to train or implement one or more systems or models. FIG. 14 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, or modified in various ways without deviating from the scope of the present disclosure. FIG. 14 is described with reference to elements/terms described with respect to other systems and figures for exemplary illustrated purposes and is not meant to be limiting. One or more portions of example method 1400 can be performed additionally, or alternatively, by other systems.

At 1402, example method 1400 can include recording natural language data using the natural language interface of a computing device, wherein the natural language interface is associated with a natural language modality. For instance, a computing device can be a user computing device that provides assistance in performing one or more tasks cooperatively with the user. For instance, a user can carry a mobile device in a medical setting (e.g., as a patient, as a medical professional) and engage the device to provide assistance in performing various tasks related to the medical setting. For instance, the user can engage the device by interacting with a natural language interface (e.g., a speech-based interface, a touch-based interface, a gesture-based interface, etc.).

At 1404, example method 1400 can include recording image data using an image capture interface of a computing device. The image capture interface can be associated with an image modality. The computing device that captures the image data can be the same device that has the natural language interface. The computing device that captures the image data can be a different device than the device that captures the natural language data. For example, a user device can include both a natural language interface (e.g., a keyboard, a microphone, etc.) and an image capture interface (e.g., a camera, an API-based connection to a remote camera, etc.). The user device can leverage image data from other devices. For instance, a user device can access image data from a dedicated imaging device (e.g., an MRI machine). A user device can execute a direct interface with the dedicated imaging device (e.g., over a network) or can access imaging data that was relayed via permanent storage on an intermediary device.

At 1406, example method 1400 can include generating a query that can include the natural language data and the image data. The query can be, for example, a query 210.

At 1408, example method 1400 can include providing the query to a multimodal machine-learned sequence processing model that generates a query response based on the query. For instance, an example model is model 108.

At 1410, example method 1400 can include rendering the query response. For instance, the computing device can receive the query response and parse the query response to extract data to render on an output interface of the computing device (e.g., a visual display, an audio driver, a haptic interface, etc.).

In some implementations of example method 1400, the multimodal machine-learned sequence processing model was trained using a training method, such as any implementation of example method 1200, any implementation of example method 1300, or any implementation of example method 1500.

For example, in some implementations of example method 1400, the multimodal machine-learned sequence processing model was trained by receiving a training query associated with a particular task domain. In some implementations of example method 1400, the multimodal machine-learned sequence processing model was trained by generating, based on the training query, a training query input that can include training query instruction data from a first modality and training query context data from a second modality. In some implementations of example method 1400, the multimodal machine-learned sequence processing model was trained by generating a combined training input that can include the training query input and a training exemplar input, wherein the training exemplar input can include training exemplar instruction data from the first modality and an exemplar context placeholder in lieu of training exemplar context data from the second modality. In some implementations of example method 1400, the multimodal machine-learned sequence processing model was trained by processing the combined training input with the multimodal machine-learned sequence processing model to generate training output data. In some implementations of example method 1400, the multimodal machine-learned sequence processing model was trained by updating one or more parameters of the machine-learned multimodal sequence processing model based on the training output data.

In some implementations, example method 1400 can include transmitting the query to a server computing system that executes the multimodal machine-learned sequence processing model. For instance, providing the query to a multimodal machine-learned sequence processing model can include providing the query to another system that executes the model (e.g., a more powerful computing system) on behalf of the computing device.

In some implementations, example method 1400 can include transmitting a runtime exemplar to the server computing system. In some implementations of example method 1400, the runtime exemplar can include the exemplar context placeholder in lieu of runtime exemplar context data from the second modality.

In some implementations of example method 1400, the runtime exemplar can be customized in association with a user account associated with the computing system. For instance, the computing device can maintain a local or remotely hosted copy of a prompt store. The exemplars in the prompt storage can be edited to conform with user preference, local regulation or guidelines, etc. These exemplars can be customized and stored in association with a user account (e.g., accessible to the user account, such as by granting access based on credentials for that user account). The computing device can retrieve such customized exemplars from storage and transmit to the server computing system. By using the placeholder in lieu of second modality data, these copies of the prompt store can be stored compactly and transmitted efficiently.

In some implementations of example method 1400, the server computing system can fetch an exemplar on behalf of the computing device (e.g., using an input orchestrator 600). The exemplar can be fetched from a global repository (e.g., default exemplars) or from a customized repository (e.g., customized to a user or accessible by the user account). By using the placeholder in lieu of second modality data, these exemplars an be stored compactly and transmitted/fetched efficiently.

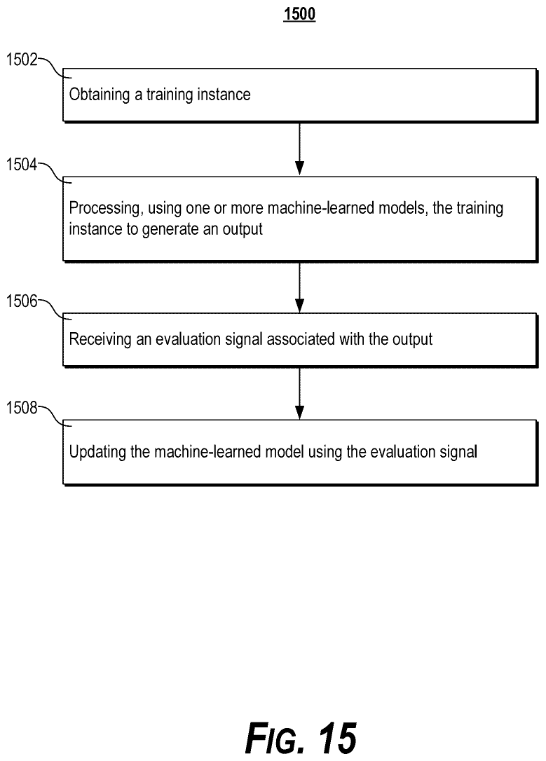

FIG. 15 depicts a flowchart of a method 1500 for training one or more machine-learned models according to aspects of the present disclosure. For instance, an example machine-learned model can include a machine-learned model 108.

One or more portion(s) of example method 1500 can be implemented by a computing system that includes one or more computing devices such as, for example, computing systems described with reference to the other figures. Each respective portion of example method 1500 can be performed by any (or any combination) of one or more computing devices. Moreover, one or more portion(s) of example method 1500 can be implemented on the hardware components of the device(s) described herein, for example, to train one or more systems or models. FIG. 15 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, or modified in various ways without deviating from the scope of the present disclosure. FIG. 15 is described with reference to elements/terms described with respect to other systems and figures for exemplary illustrated purposes and is not meant to be limiting. One or more portions of example method 1500 can be performed additionally, or alternatively, by other systems.

At 1502, example method 1500 can include obtaining a training instance. A set of training data can include a plurality of training instances divided between multiple datasets (e.g., a training dataset, a validation dataset, or testing dataset). A training instance can be labeled or unlabeled. Although referred to in example method 1500 as a “training” instance, it is to be understood that runtime inferences can form training instances when a model is trained using an evaluation of the model's performance on that runtime instance (e.g., online training/learning). Example data types for the training instance and various tasks associated therewith are described throughout the present disclosure, such as the MultiMedBench datasets.

At 1504, example method 1500 can include processing, using one or more machine-learned models, the training instance to generate an output. The output can be directly obtained from the one or more machine-learned models or can be a downstream result of a chain of processing operations that includes an output of the one or more machine-learned models.

At 1506, example method 1500 can include receiving an evaluation signal associated with the output. The evaluation signal can be obtained using a loss function. Various determinations of loss can be used, such as mean squared error, likelihood loss, cross entropy loss, hinge loss, contrastive loss, or various other loss functions. The evaluation signal can be computed using known ground-truth labels (e.g., supervised learning), predicted or estimated labels (e.g., semi- or self-supervised learning), or without labels (e.g., unsupervised learning). The evaluation signal can be a reward (e.g., for reinforcement learning). The reward can be computed using a machine-learned reward model configured to generate rewards based on output(s) received. The reward can be computed using feedback data describing human feedback on the output(s).

At 1508, example method 1500 can include updating the machine-learned model using the evaluation signal. For example, values for parameters of the machine-learned model(s) can be learned, in some embodiments, using various training or learning techniques, such as, for example, backwards propagation. For example, the evaluation signal can be backpropagated from the output (or another source of the evaluation signal) through the machine-learned model(s) to update one or more parameters of the model(s) (e.g., based on a gradient of the evaluation signal with respect to the parameter value(s)). For example, system(s) containing one or more machine-learned models can be trained in an end-to-end manner. Gradient descent techniques can be used to iteratively update the parameters over a number of training iterations. In some implementations, performing backwards propagation of errors can include performing truncated backpropagation through time. Example method 1500 can include implementing a number of generalization techniques (e.g., weight decays, dropouts, etc.) to improve the generalization capability of the models being trained.

In some implementations, example method 1500 can be implemented for training a machine-learned model from an initialized state to a fully trained state (e.g., when the model exhibits a desired performance profile, such as based on accuracy, precision, recall, etc.).

In some implementations, example method 1500 can be implemented for particular stages of a training procedure. For instance, in some implementations, example method 1500 can be implemented for pre-training a machine-learned model. Pre-training can include, for instance, large-scale training over potentially noisy data to achieve a broad base of performance levels across a variety of tasks/data types. In some implementations, example method 1500 can be implemented for fine-tuning a machine-learned model. Fine-tuning can include, for instance, smaller-scale training on higher-quality (e.g., labeled, curated, etc.) data. Fine-tuning can affect all or a portion of the parameters of a machine-learned model. For example, various portions of the machine-learned model can be “frozen” for certain training stages. For example, parameters associated with an embedding space can be “frozen” during fine-tuning (e.g., to retain information learned from a broader domain(s) than present in the fine-tuning dataset(s)). An example fine-tuning approach includes reinforcement learning. Reinforcement learning can be based on user feedback on model performance during use.

### Example Machine-Learned Models

FIG. 16 is a block diagram of an example processing flow for using machine-learned model(s) 1 to process input(s) 2 to generate output(s) 3.

Machine-learned model(s) 1 can be or include any one of or any part of machine-learned models referenced with respect to system 100, or any other system referenced herein. For example, machine-learned model 108 can be a machine-learned model 1. Any model or learned component described in any of the Figures (e.g., model trainer 112, Substitution Operator 220, Input Encoder 228, Input Orchestrator 600, Modality-Specific Input Encoder 802, Modality-Specific Input Encoder 804, Human Feedback Interface 1000, Human Feedback Interface 1100, etc.) can be, include, or be implemented using a machine-learned model 1. Features and variations described herein with respect to machine-learned model 1 are to be understood as describing features and variations of any of such models and components described herein. Where this description references machine-learned model 1 it is to be understood that implementations of each of the other models and components described herein are implicitly referenced and represented thereby. Any variation or combination of features of machine-learned model 1 can be implemented in or for any model or learned component described in any of the Figures.

Machine-learned model(s) 1 can be or include one or multiple machine-learned models or model components. Example machine-learned models can include neural networks (e.g., deep neural networks). Example machine-learned models can include non-linear models or linear models. Example machine-learned models can use other architectures in lieu of or in addition to neural networks. Example machine-learned models can include decision tree based models, support vector machines, hidden Markov models, Bayesian networks, linear regression models, k-means clustering models, etc.

Example neural networks can include feed-forward neural networks, recurrent neural networks (RNNs), including long short-term memory (LSTM) based recurrent neural networks, convolutional neural networks (CNNs), diffusion models, generative-adversarial networks, or other forms of neural networks. Example neural networks can be deep neural networks. Some example machine-learned models can leverage an attention mechanism such as self-attention. For example, some example machine-learned models can include multi-headed self-attention models.

Machine-learned model(s) 1 can include a single or multiple instances of the same model configured to operate on data from input(s) 2. Machine-learned model(s) 1 can include an ensemble of different models that can cooperatively interact to process data from input(s) 2. For example, machine-learned model(s) 1 can employ a mixture-of-experts structure. See, e.g., Zhou et al., Mixture-of-Experts with Expert Choice Routing, ARXIV: 2202.09368v2 (Oct. 14, 2022).

Input(s) 2 can generally include or otherwise represent various types of data. Input(s) 2 can include one type or many different types of data. Output(s) 3 can be data of the same type(s) or of different types of data as compared to input(s) 2. Output(s) 3 can include one type or many different types of data.

Example data types for input(s) 2 or output(s) 3 include natural language text data, software code data (e.g., source code, object code, machine code, or any other form of computer-readable instructions or programming languages), machine code data (e.g., binary code, assembly code, or other forms of machine-readable instructions that can be executed directly by a computer's central processing unit), assembly code data (e.g., low-level programming languages that use symbolic representations of machine code instructions to program a processing unit), genetic data or other chemical or biochemical data, image data, audio data, audiovisual data, haptic data, biometric data, medical data, financial data, statistical data, geographical data, astronomical data, historical data, sensor data generally (e.g., digital or analog values, such as voltage or other absolute or relative level measurement values from a real or artificial input, such as from an audio sensor, light sensor, displacement sensor, etc.), and the like. Data can be raw or processed and can be in any format or schema.

In multimodal inputs 2 or outputs 3, example combinations of data types include image data and audio data, image data and natural language data, natural language data and software code data, image data and biometric data, sensor data and medical data, etc. It is to be understood that any combination of data types in an input 2 or an output 3 can be present.

An example input 2 can include one or multiple data types, such as the example data types noted above. An example output 3 can include one or multiple data types, such as the example data types noted above. The data type(s) of input 2 can be the same as or different from the data type(s) of output 3. It is to be understood that the example data types noted above are provided for illustrative purposes only. Data types contemplated within the scope of the present disclosure are not limited to those examples noted above.

### Example Machine-Learned Sequence Processing Models

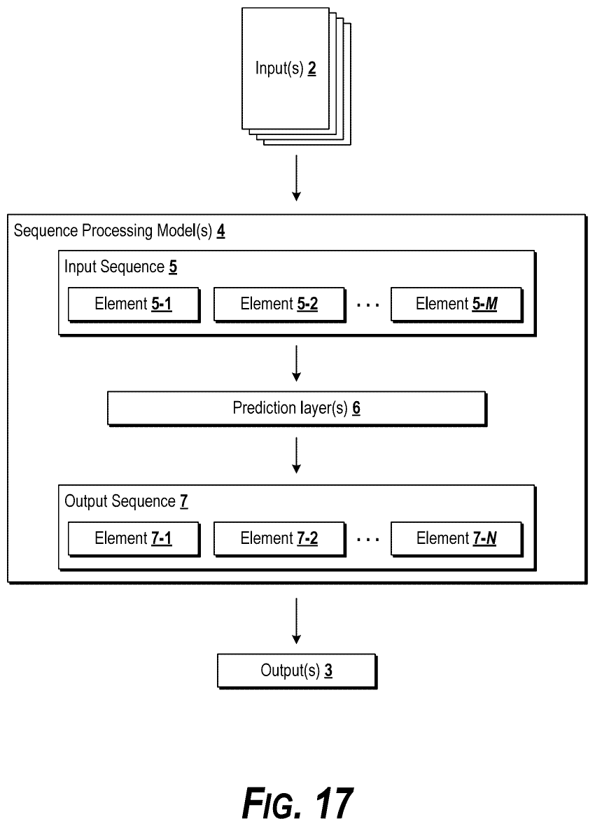

FIG. 17 is a block diagram of an example implementation of an example machine-learned model configured to process sequences of information. For instance, an example implementation of machine-learned model(s) 1 can include machine-learned sequence processing model(s) 4. An example system can pass input(s) 2 to sequence processing model(s) 4. Sequence processing model(s) 4 can include one or more machine-learned components. Sequence processing model(s) 4 can process the data from input(s) 2 to obtain an input sequence 5. Input sequence 5 can include one or more input elements 5-1, 5-2, . . . , 5-M, etc. obtained from input(s) 2. Sequence processing model 4 can process input sequence 5 using prediction layer(s) 6 to generate an output sequence 7. Output sequence 7 can include one or more output elements 7-1, 7-2, . . . , 7-N, etc. generated based on input sequence 5. The system can generate output(s) 3 based on output sequence 7.

Sequence processing model(s) 4 can include one or multiple machine-learned model components configured to ingest, generate, or otherwise reason over sequences of information. For example, some example sequence processing models in the text domain are referred to as “Large Language Models,” or LLMs. See, e.g., PaLM 2 Technical Report, GOOGLE, https://ai.google/static/documents/palm2techreport.pdf (n.d.). Other example sequence processing models can operate in other domains, such as image domains, see, e.g., Dosovitskiy et al., An Image is Worth 16×16 Words: Transformers for Image Recognition at Scale, ARXIV: 2010.11929v2 (Jun. 3, 2021), audio domains, see, e.g., Agostinelli et al., MusicLM: Generating Music From Text, ARXIV: 2301.11325v1 (Jan. 26, 2023), biochemical domains, see, e.g., Jumper et al., Highly accurate protein structure prediction with AlphaFold, 596 Nature 583 (Aug. 26, 2021), by way of example. Sequence processing model(s) 4 can process one or multiple types of data simultaneously. Sequence processing model(s) 4 can include relatively large models (e.g., more parameters, computationally expensive, etc.), relatively small models (e.g., fewer parameters, computationally lightweight, etc.), or both.

In general, sequence processing model(s) 4 can obtain input sequence 5 using data from input(s) 2. For instance, input sequence 5 can include a representation of data from input(s) 2 in a format understood by sequence processing model(s) 4. One or more machine-learned components of sequence processing model(s) 4 can ingest the data from input(s) 2, parse the data into pieces compatible with the processing architectures of sequence processing model(s) 4 (e.g., via “tokenization”), and project the pieces into an input space associated with prediction layer(s) 6 (e.g., via “embedding”).

Sequence processing model(s) 4 can ingest the data from input(s) 2 and parse the data into a sequence of elements to obtain input sequence 5. For example, a portion of input data from input(s) 2 can be broken down into pieces that collectively represent the content of the portion of the input data. The pieces can provide the elements of the sequence.

Elements 5-1, 5-2, . . . , 5-M can represent, in some cases, building blocks for capturing or expressing meaningful information in a particular data domain. For instance, the elements can describe “atomic units” across one or more domains. For example, for textual input source(s), the elements can correspond to groups of one or more words or sub-word components, such as sets of one or more characters.

For example, elements 5-1, 5-2, . . . , 5-M can represent tokens obtained using a tokenizer. For instance, a tokenizer can process a given portion of an input source and output a series of tokens (e.g., corresponding to input elements 5-1, 5-2, . . . , 5-M) that represent the portion of the input source. Various approaches to tokenization can be used. For instance, textual input source(s) can be tokenized using a byte-pair encoding (BPE) technique. See, e.g., Kudo et al., SentencePiece: A simple and language independent subword tokenizer and detokenizer for Neural Text Processing, PROCEEDINGS OF THE 2018 CONFERENCE ON EMPIRICAL METHODS IN NATURAL LANGUAGE PROCESSING (System Demonstrations), pages 66-71 (Oct. 31-Nov. 4, 2018), https://aclanthology.org/D18-2012.pdf. Image-based input source(s) can be tokenized by extracting and serializing patches from an image.

In general, arbitrary data types can be serialized and processed into input sequence 5. It is to be understood that element(s) 5-1, 5-2, . . . , 5-M depicted in FIG. 17 can be the tokens or can be the embedded representations thereof.

Prediction layer(s) 6 can predict one or more output elements 7-1, 7-2, . . . , 7-N based on the input elements. Prediction layer(s) 6 can include one or more machine-learned model architectures, such as one or more layers of learned parameters that manipulate and transform the input(s) to extract higher-order meaning from, and relationships between, input element(s) 5-1, 5-2, . . . , 5-M. In this manner, for instance, example prediction layer(s) 6 can predict new output element(s) in view of the context provided by input sequence 5.

Prediction layer(s) 6 can evaluate associations between portions of input sequence 5 and a particular output element. These associations can inform a prediction of the likelihood that a particular output follows the input context. For example, consider the textual snippet, “The carpenter's toolbox was small and heavy. It was full of ______.” Example prediction layer(s) 6 can identify that “It” refers back to “toolbox” by determining a relationship between the respective embeddings. Example prediction layer(s) 6 can also link “It” to the attributes of the toolbox, such as “small” and “heavy.” Based on these associations, prediction layer(s) 6 can, for instance, assign a higher probability to the word “nails” than to the word “sawdust.”

A transformer is an example architecture that can be used in prediction layer(s) 4. See, e.g., Vaswani et al., Attention Is All You Need, ARXIV: 1706.03762v7 (Aug. 2, 2023). A transformer is an example of a machine-learned model architecture that uses an attention mechanism to compute associations between items within a context window. The context window can include a sequence that contains input sequence 5 and potentially one or more output element(s) 7-1, 7-2, . . . , 7-N. A transformer block can include one or more attention layer(s) and one or more post-attention layer(s) (e.g., feedforward layer(s), such as a multi-layer perceptron).

Prediction layer(s) 6 can include other machine-learned model architectures in addition to or in lieu of transformer-based architectures. For example, recurrent neural networks (RNNs) and long short-term memory (LSTM) models can also be used, as well as convolutional neural networks (CNNs). In general, prediction layer(s) 6 can leverage various kinds of artificial neural networks that can understand or generate sequences of information.

Output sequence 7 can include or otherwise represent the same or different data types as input sequence 5. For instance, input sequence 5 can represent textual data, and output sequence 7 can represent textual data. Input sequence 5 can represent image, audio, or audiovisual data, and output sequence 7 can represent textual data (e.g., describing the image, audio, or audiovisual data). It is to be understood that prediction layer(s) 6, and any other interstitial model components of sequence processing model(s) 4, can be configured to receive a variety of data types in input sequence(s) 5 and output a variety of data types in output sequence(s) 7.

Output sequence 7 can have various relationships to input sequence 5. Output sequence 7 can be a continuation of input sequence 5. Output sequence 7 can be complementary to input sequence 5. Output sequence 7 can translate, transform, augment, or otherwise modify input sequence 5. Output sequence 7 can answer, evaluate, confirm, or otherwise respond to input sequence 5. Output sequence 7 can implement (or describe instructions for implementing) an instruction provided via input sequence 5.

Output sequence 7 can be generated autoregressively. For instance, for some applications, an output of one or more prediction layer(s) 6 can be passed through one or more output layers (e.g., softmax layer) to obtain a probability distribution over an output vocabulary (e.g., a textual or symbolic vocabulary) conditioned on a set of input elements in a context window. In this manner, for instance, output sequence 7 can be autoregressively generated by sampling a likely next output element, adding that element to the context window, and re-generating the probability distribution based on the updated context window, and sampling a likely next output element, and so forth.

Output sequence 7 can also be generated non-autoregressively. For instance, multiple output elements of output sequence 7 can be predicted together without explicit sequential conditioning on each other. See, e.g., Saharia et al., Non-Autoregressive Machine Translation with Latent Alignments, ARXIV: 2004.07437v3 (Nov. 16, 2020).

Output sequence 7 can include one or multiple portions or elements. In an example content generation configuration, output sequence 7 can include multiple elements corresponding to multiple portions of a generated output sequence (e.g., a textual sentence, values of a discretized waveform, computer code, etc.). In an example classification configuration, output sequence 7 can include a single element associated with a classification output. For instance, an output “vocabulary” can include a set of classes into which an input sequence is to be classified. For instance, a vision transformer block can pass latent state information to a multilayer perceptron that outputs a likely class value associated with an input image.

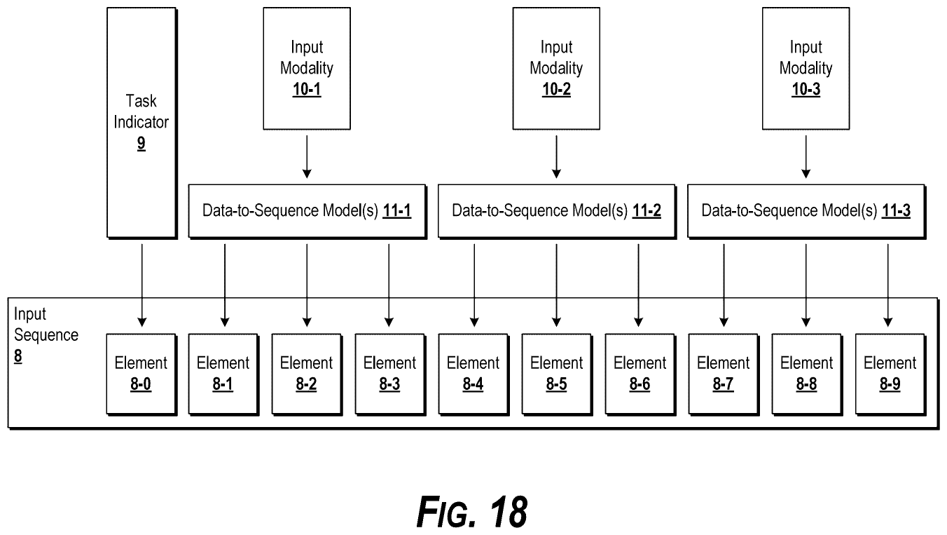

FIG. 18 is a block diagram of an example technique for populating an example input sequence 8. Input sequence 8 can include various functional elements that form part of the model infrastructure, such as an element 8-0 obtained from a task indicator 9 that signals to any model(s) that process input sequence 8 that a particular task is being performed (e.g., to help adapt a performance of the model(s) to that particular task). Input sequence 8 can include various data elements from different data modalities. For instance, an input modality 10-1 can include one modality of data. A data-to-sequence model 11-1 can process data from input modality 10-1 to project the data into a format compatible with input sequence 8 (e.g., one or more vectors dimensioned according to the dimensions of input sequence 8) to obtain elements 8-1, 8-2, 8-3. Another input modality 10-2 can include a different modality of data. A data-to-sequence model 11-2 can project data from input modality 10-2 into a format compatible with input sequence 8 to obtain elements 8-4, 8-5, 8-6. Another input modality 10-3 can include yet another different modality of data. A data-to-sequence model 11-3 can project data from input modality 10-3 into a format compatible with input sequence 8 to obtain elements 8-7, 8-8, 8-9.

Input sequence 8 can be the same as or different from input sequence 5. Input sequence 8 can be a multimodal input sequence that contains elements that represent data from different modalities using a common dimensional representation. For instance, an embedding space can have P dimensions. Input sequence 8 can be configured to contain a plurality of elements that have P dimensions. In this manner, for instance, example implementations can facilitate information extraction and reasoning across diverse data modalities by projecting data into elements in the same embedding space for comparison, combination, or other computations therebetween.

For example, elements 8-0, . . . , 8-9 can indicate particular locations within a multidimensional embedding space. Some elements can map to a set of discrete locations in the embedding space. For instance, elements that correspond to discrete members of a predetermined vocabulary of tokens can map to discrete locations in the embedding space that are associated with those tokens. Other elements can be continuously distributed across the embedding space. For instance, some data types can be broken down into continuously defined portions (e.g., image patches) that can be described using continuously distributed locations within the embedding space.

In some implementations, the expressive power of the embedding space may not be limited to meanings associated with any particular set of tokens or other building blocks. For example, a continuous embedding space can encode a spectrum of high-order information. An individual piece of information (e.g., a token) can map to a particular point in that space: for instance, a token for the word “dog” can be projected to an embedded value that points to a particular location in the embedding space associated with canine-related information. Similarly, an image patch of an image of a dog on grass can also be projected into the embedding space. In some implementations, the projection of the image of the dog can be similar to the projection of the word “dog” while also having similarity to a projection of the word “grass,” while potentially being different from both. In some implementations, the projection of the image patch may not exactly align with any single projection of a single word. In some implementations, the projection of the image patch can align with a combination of the projections of the words “dog” and “grass.” In this manner, for instance, a high-order embedding space can encode information that can be independent of data modalities in which the information is expressed.

Task indicator 9 can include a model or model component configured to identify a task being performed and inject, into input sequence 8, an input value represented by element 8-0 that signals which task is being performed. For instance, the input value can be provided as a data type associated with an input modality and projected along with that input modality (e.g., the input value can be a textual task label that is embedded along with other textual data in the input; the input value can be a pixel-based representation of a task that is embedded along with other image data in the input; etc.). The input value can be provided as a data type that differs from or is at least independent from other input(s). For instance, the input value represented by element 8-0 can be a learned within a continuous embedding space.

Input modalities 10-1, 10-2, and 10-3 can be associated with various different data types (e.g., as described above with respect to input(s) 2 and output(s) 3).

Data-to-sequence models 11-1, 11-2, and 11-3 can be the same or different from each other. Data-to-sequence models 11-1, 11-2, and 11-3 can be adapted to each respective input modality 10-1, 10-2, and 10-3. For example, a textual data-to-sequence model can subdivide a portion of input text and project the subdivisions into element(s) in input sequence 8 (e.g., elements 8-1, 8-2, 8-3, etc.). An image data-to-sequence model can subdivide an input image and project the subdivisions into element(s) in input sequence 8 (e.g., elements 8-4, 8-5, 8-6, etc.). An arbitrary datatype data-to-sequence model can subdivide an input of that arbitrary datatype and project the subdivisions into element(s) in input sequence 8 (e.g., elements 8-7, 8-8, 8-9, etc.).

Data-to-sequence models 11-1, 11-2, and 11-3 can form part of machine-learned sequence processing model(s) 4. Data-to-sequence models 11-1, 11-2, and 11-3 can be jointly trained with or trained independently from machine-learned sequence processing model(s) 4. Data-to-sequence models 11-1, 11-2, and 11-3 can be trained end-to-end with machine-learned sequence processing model(s) 4.

An example data-to-sequence model is input encoder 228. Data-to-sequence models 11-1, 11-2, and 11-3 can respectively process different modalities of a query 210 to populate a query input portion. An example query input portion is Input sequence 8.

### Example Machine-Learned Model Development Platform

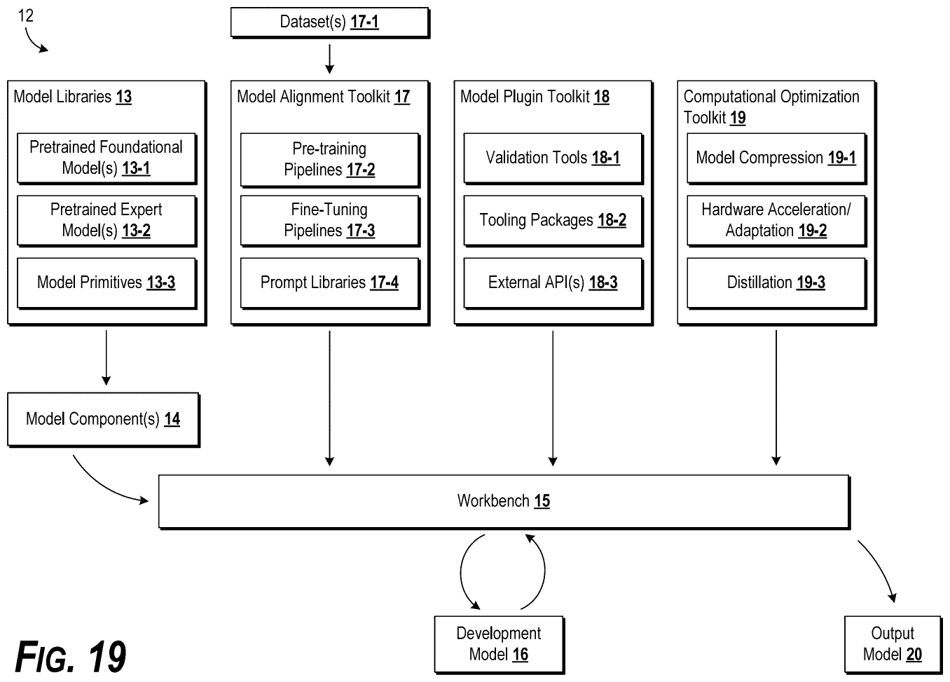

FIG. 19 is a block diagram of an example model development platform 12 that can facilitate creation, adaptation, and refinement of example machine-learned models (e.g., machine-learned model(s) 1, sequence processing model(s) 4, etc.). Model development platform 12 can provide a number of different toolkits that developer systems can employ in the development of new or adapted machine-learned models.

Model development platform 12 can provide one or more model libraries 13 containing building blocks for new models. Model libraries 13 can include one or more pre-trained foundational models 13-1, which can provide a backbone of processing power across various tasks. Model libraries 13 can include one or more pre-trained expert models 13-2, which can be focused on performance in particular domains of expertise. Model libraries 13 can include various model primitives 13-3, which can provide low-level architectures or components (optionally pre-trained), which can be assembled in various arrangements as desired.

Model development platform 12 can receive selections of various model components 14. Model development platform 12 can pass selected model components 14 to a workbench 15 that combines selected model components 14 into a development model 16.

Workbench 15 can facilitate further refinement and adaptation of development model 16 by leveraging a number of different toolkits integrated with model development platform 12. For example, workbench 15 can facilitate alignment of the development model 16 with a desired performance profile on various tasks using a model alignment toolkit 17.

Model alignment toolkit 17 can provide a number of tools for causing development model 16 to generate outputs aligned with desired behavioral characteristics. Alignment can include increasing an accuracy, precision, recall, etc. of model outputs. Alignment can include enforcing output styles, schema, or other preferential characteristics of model outputs. Alignment can be general or domain-specific. For instance, a pre-trained foundational model 13-1 can begin with an initial level of performance across multiple domains. Alignment of the pre-trained foundational model 13-1 can include improving a performance in a particular domain of information or tasks (e.g., even at the expense of performance in another domain of information or tasks).

Model alignment toolkit 17 can integrate one or more dataset(s) 17-1 for aligning development model 16. Curated dataset(s) 17-1 can include labeled or unlabeled training data. Dataset(s) 17-1 can be obtained from public domain datasets. Dataset(s) 17-1 can be obtained from private datasets associated with one or more developer system(s) for the alignment of bespoke machine-learned model(s) customized for private use-cases.

Pre-training pipelines 17-2 can include a machine-learned model training workflow configured to update development model 16 over large-scale, potentially noisy datasets. For example, pre-training can leverage unsupervised learning techniques (e.g., de-noising, etc.) to process large numbers of training instances to update model parameters from an initialized state and achieve a desired baseline performance. Pre-training pipelines 17-2 can leverage unlabeled datasets in dataset(s) 17-1 to perform pre-training. Workbench 15 can implement a pre-training pipeline 17-2 to pre-train development model 16.

Fine-tuning pipelines 17-3 can include a machine-learned model training workflow configured to refine the model parameters of development model 16 with higher-quality data. Fine-tuning pipelines 17-3 can update development model 16 by conducting supervised training with labeled dataset(s) in dataset(s) 17-1. Fine-tuning pipelines 17-3 can update development model 16 by conducting reinforcement learning using reward signals from user feedback signals. Workbench 15 can implement a fine-tuning pipeline 17-3 to fine-tune development model 16.

Prompt libraries 17-4 can include sets of inputs configured to induce behavior aligned with desired performance criteria. Prompt libraries 17-4 can include few-shot prompts (e.g., inputs providing examples of desired model outputs for prepending to a desired runtime query), chain-of-thought prompts (e.g., inputs providing step-by-step reasoning within the exemplars to facilitate thorough reasoning by the model), and the like.

Example prompts can be retrieved from an available repository of prompt libraries 17-4. Example prompts can be contributed by one or more developer systems using workbench 15.

In some implementations, pre-trained or fine-tuned models can achieve satisfactory performance without exemplars in the inputs. For instance, zero-shot prompts can include inputs that lack exemplars. Zero-shot prompts can be within a domain within a training dataset or outside of the training domain(s).

Prompt libraries 17-4 can include one or more prompt engineering tools. Prompt engineering tools can provide workflows for retrieving or learning optimized prompt values. Prompt engineering tools can facilitate directly learning prompt values (e.g., input element values) based one or more training iterations. Workbench 15 can implement prompt engineering tools in development model 16.

Prompt libraries 17-4 can include pipelines for prompt generation. For example, inputs can be generated using development model 16 itself or other machine-learned models. In this manner, for instance, a first model can process information about a task and output a input for a second model to process in order to perform a step of the task. The second model can be the same as or different from the first model. Workbench 15 can implement prompt generation pipelines in development model 16.

Prompt libraries 17-4 can include pipelines for context injection. For instance, a performance of development model 16 on a particular task can improve if provided with additional context for performing the task. Prompt libraries 17-4 can include software components configured to identify desired context, retrieve the context from an external source (e.g., a database, a sensor, etc.), and add the context to the input prompt. Workbench 15 can implement context injection pipelines in development model 16.

Although various training examples described herein with respect to model development platform 12 refer to “pre-training” and “fine-tuning,” it is to be understood that model alignment toolkit 17 can generally support a wide variety of training techniques adapted for training a wide variety of machine-learned models. Example training techniques can correspond to the example training method 1500 described above.

Model development platform 12 can include a model plugin toolkit 18. Model plugin toolkit 18 can include a variety of tools configured for augmenting the functionality of a machine-learned model by integrating the machine-learned model with other systems, devices, and software components. For instance, a machine-learned model can use tools to increase performance quality where appropriate. For instance, deterministic tasks can be offloaded to dedicated tools in lieu of probabilistically performing the task with an increased risk of error. For instance, instead of autoregressively predicting the solution to a system of equations, a machine-learned model can recognize a tool to call for obtaining the solution and pass the system of equations to the appropriate tool. The tool can be a traditional system of equations solver that can operate deterministically to resolve the system of equations. The output of the tool can be returned in response to the original query. In this manner, tool use can allow some example models to focus on the strengths of machine-learned models—e.g., understanding an intent in an unstructured request for a task—while augmenting the performance of the model by offloading certain tasks to a more focused tool for rote application of deterministic algorithms to a well-defined problem.

Model plugin toolkit 18 can include validation tools 18-1. Validation tools 18-1 can include tools that can parse and confirm output(s) of a machine-learned model. Validation tools 18-1 can include engineered heuristics that establish certain thresholds applied to model outputs. For example, validation tools 18-1 can ground the outputs of machine-learned models to structured data sources (e.g., to mitigate “hallucinations”).

Model plugin toolkit 18 can include tooling packages 18-2 for implementing one or more tools that can include scripts or other executable code that can be executed alongside development model 16. Tooling packages 18-2 can include one or more inputs configured to cause machine-learned model(s) to implement the tools (e.g., few-shot prompts that induce a model to output tool calls in the proper syntax, etc.). Tooling packages 18-2 can include, for instance, fine-tuning training data for training a model to use a tool.

Model plugin toolkit 18 can include interfaces for calling external application programming interfaces (APIs) 18-3. For instance, in addition to or in lieu of implementing tool calls or tool code directly with development model 16, development model 16 can be aligned to output instruction that initiate API calls to send or obtain data via external systems.

Model plugin toolkit 18 can integrate with prompt libraries 17-4 to build a catalog of available tools for use with development model 16. For instance, a model can receive, in an input, a catalog of available tools, and the model can generate an output that selects a tool from the available tools and initiates a tool call for using the tool.

Model development platform 12 can include a computational optimization toolkit 19 for optimizing a computational performance of development model 16. For instance, tools for model compression 19-1 can allow development model 16 to be reduced in size while maintaining a desired level of performance. For instance, model compression 19-1 can include quantization workflows, weight pruning and sparsification techniques, etc. Tools for hardware acceleration 19-2 can facilitate the configuration of the model storage and execution formats to operate optimally on different hardware resources. For instance, hardware acceleration 19-2 can include tools for optimally sharding models for distributed processing over multiple processing units for increased bandwidth, lower unified memory requirements, etc. Tools for distillation 19-3 can provide for the training of lighter-weight models based on the knowledge encoded in development model 16. For instance, development model 16 can be a highly performant, large machine-learned model optimized using model development platform 12. To obtain a lightweight model for running in resource-constrained environments, a smaller model can be a “student model” that learns to imitate development model 16 as a “teacher model.” In this manner, for instance, the investment in learning the parameters and configurations of development model 16 can be efficiently transferred to a smaller model for more efficient inference.

Workbench 15 can implement one, multiple, or none of the toolkits implemented in model development platform 12. Workbench 15 can output an output model 20 based on development model 16. Output model 20 can be a deployment version of development model 16. Output model 20 can be a development or training checkpoint of development model 16. Output model 20 can be a distilled, compressed, or otherwise optimized version of development model 16.

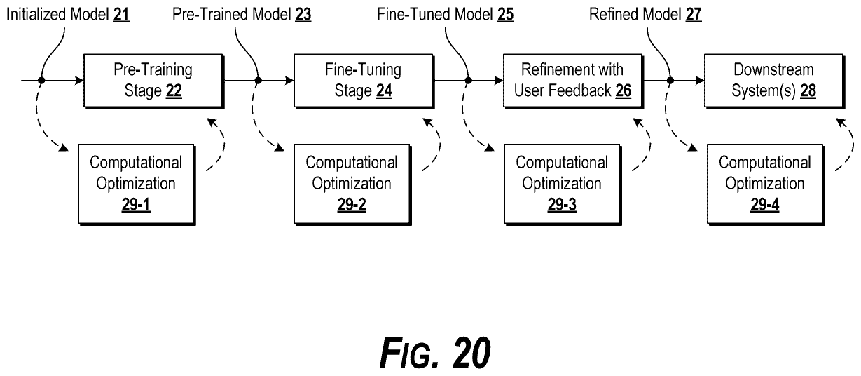

FIG. 20 is a block diagram of an example training flow for training a machine-learned development model 16. One or more portion(s) of the example training flow can be implemented by a computing system that includes one or more computing devices such as, for example, computing systems described with reference to the other figures. Each respective portion of the example training flow can be performed by any (or any combination) of one or more computing devices. Moreover, one or more portion(s) of the example training flow can be implemented on the hardware components of the device(s) described herein, for example, to train one or more systems or models. FIG. 20 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, or modified in various ways without deviating from the scope of the present disclosure. FIG. 20 is described with reference to elements/terms described with respect to other systems and figures for exemplary illustrated purposes and is not meant to be limiting. One or more portions of the example training flow can be performed additionally, or alternatively, by other systems.

Initially, development model 16 can persist in an initial state as an initialized model 21. Development model 16 can be initialized with weight values. Initial weight values can be random or based on an initialization schema. Initial weight values can be based on prior pre-training for the same or for a different model.

Initialized model 21 can undergo pre-training in a pre-training stage 22. Pre-training stage 22 can be implemented using one or more pre-training pipelines 17-2 over data from dataset(s) 17-1. Pre-training can be omitted, for example, if initialized model 21 is already pre-trained (e.g., development model 16 contains, is, or is based on a pre-trained foundational model or an expert model).

Pre-trained model 23 can then be a new version of development model 16, which can persist as development model 16 or as a new development model. Pre-trained model 23 can be the initial state if development model 16 was already pre-trained. Pre-trained model 23 can undergo fine-tuning in a fine-tuning stage 24. Fine-tuning stage 24 can be implemented using one or more fine-tuning pipelines 17-3 over data from dataset(s) 17-1. Fine-tuning can be omitted, for example, if a pre-trained model as satisfactory performance, if the model was already fine-tuned, or if other tuning approaches are preferred.

Fine-tuned model 29 can then be a new version of development model 16, which can persist as development model 16 or as a new development model. Fine-tuned model 29 can be the initial state if development model 16 was already fine-tuned. Fine-tuned model 29 can undergo refinement with user feedback 26. For instance, refinement with user feedback 26 can include reinforcement learning, optionally based on human feedback from human users of fine-tuned model 25. As reinforcement learning can be a form of fine-tuning, it is to be understood that fine-tuning stage 24 can subsume the stage for refining with user feedback 26. Refinement with user feedback 26 can produce a refined model 27. Refined model 27 can be output to downstream system(s) 28 for deployment or further development.

In some implementations, computational optimization operations can be applied before, during, or after each stage. For instance, initialized model 21 can undergo computational optimization 29-1 (e.g., using computational optimization toolkit 19) before pre-training stage 22. Pre-trained model 23 can undergo computational optimization 29-2 (e.g., using computational optimization toolkit 19) before fine-tuning stage 24. Fine-tuned model 25 can undergo computational optimization 29-3 (e.g., using computational optimization toolkit 19) before refinement with user feedback 26. Refined model 27 can undergo computational optimization 29-4 (e.g., using computational optimization toolkit 19) before output to downstream system(s) 28. Computational optimization(s) 29-1, . . . , 29-4 can all be the same, all be different, or include at least some different optimization techniques.

### Example Machine-Learned Model Inference System

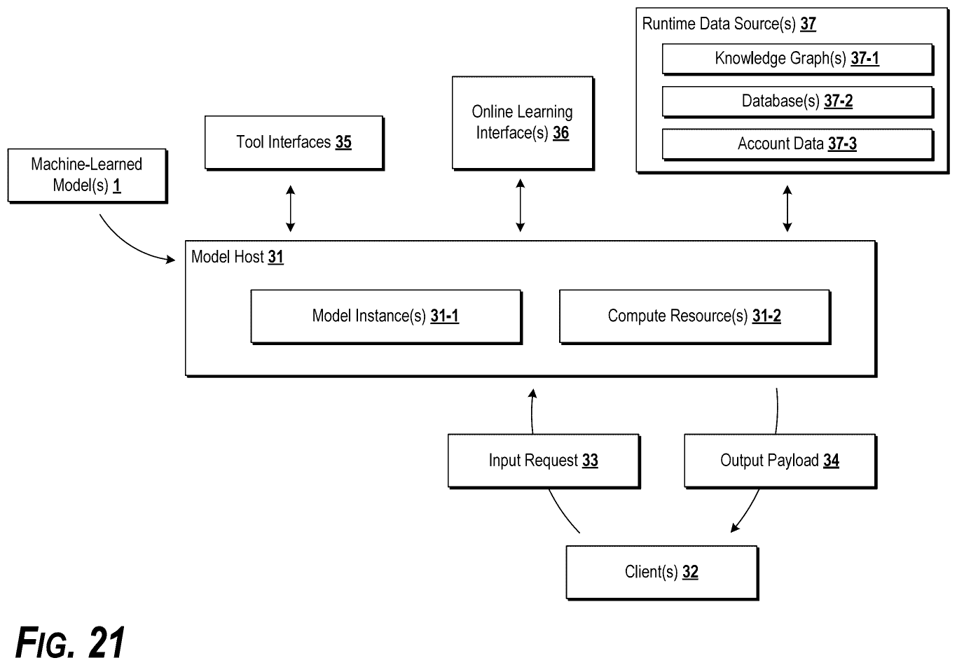

FIG. 21 is a block diagram of an inference system for operating one or more machine-learned model(s) 1 to perform inference (e.g., for training, for deployment, etc.). A model host 31 can receive machine-learned model(s) 1. Model host 31 can host one or more model instance(s) 31-1, which can be one or multiple instances of one or multiple models. Model host 31 can host model instance(s) 31-1 using available compute resources 31-2 associated with model host 31.

Model host 31 can perform inference on behalf of one or more client(s) 32. Client(s) 32 can transmit an input request 33 to model host 31. Using input request 33, model host 31 can obtain input(s) 2 for input to machine-learned model(s) 1. Machine-learned model(s) 1 can process input(s) 2 to generate output(s) 3. Using output(s) 3, model host 31 can return an output payload 34 for responding to input request 33 from client(s) 32. Output payload 34 can include or be based on output(s) 3.

Model host 31 can leverage various other resources and tools to augment the inference task. For instance, model host 31 can communicate with tool interfaces 35 to facilitate tool use by model instance(s) 31-1. Tool interfaces 35 can include local or remote APIs. Tool interfaces 35 can include integrated scripts or other software functionality. Model host 31 can engage online learning interface(s) 36 to facilitate ongoing improvements to machine-learned model(s) 1. For instance, online learning interface(s) 36 can be used within reinforcement learning loops to retrieve user feedback on inferences served by model host 31. Model host 31 can access runtime data source(s) 37 for augmenting input(s) 2 with additional contextual information. For instance, runtime data source(s) 37 can include a knowledge graph 37-1 that facilitates structured information retrieval for information associated with input request(s) 33 (e.g., a search engine service). Runtime data source(s) 37 can include public or private, external or local database(s) 37-2 that can store information associated with input request(s) 33 for augmenting input(s) 2. Runtime data source(s) 37 can include account data 37-3 which can be retrieved in association with a user account corresponding to a client 32 for customizing the behavior of model host 31 accordingly.

Model host 31 can be implemented by one or multiple computing devices or systems. Client(s) 2 can be implemented by one or multiple computing devices or systems, which can include computing devices or systems shared with model host 31.

For example, model host 31 can operate on a server system that provides a machine-learning service to client device(s) that operate client(s) 32 (e.g., over a local or wide-area network). Client device(s) can be end-user devices used by individuals. Client device(s) can be server systems that operate client(s) 32 to provide various functionality as a service to downstream end-user devices.

In some implementations, model host 31 can operate on a same device or system as client(s) 32. Model host 31 can be a machine-learning service that runs on-device to provide machine-learning functionality to one or multiple applications operating on a client device, which can include an application implementing client(s) 32. Model host 31 can be a part of a same application as client(s) 32. For instance, model host 31 can be a subroutine or method implemented by one part of an application, and client(s) 32 can be another subroutine or method that engages model host 31 to perform inference functions within the application. It is to be understood that model host 31 and client(s) 32 can have various different configurations.

Model instance(s) 31-1 can include one or more machine-learned models that are available for performing inference. Model instance(s) 31-1 can include weights or other model components that are stored on in persistent storage, temporarily cached, or loaded into high-speed memory. Model instance(s) 31-1 can include multiple instance(s) of the same model (e.g., for parallel execution of more requests on the same model). Model instance(s) 31-1 can include instance(s) of different model(s). Model instance(s) 31-1 can include cached intermediate states of active or inactive model(s) used to accelerate inference of those models. For instance, an inference session with a particular model may generate significant amounts of computational results that can be re-used for future inference runs (e.g., using a KV cache for transformer-based models). These computational results can be saved in association with that inference session so that session can be executed more efficiently when resumed.

Compute resource(s) 31-2 can include one or more processors (central processing units, graphical processing units, tensor processing units, machine-learning accelerators, etc.) connected to one or more memory devices. Compute resource(s) 31-2 can include a dynamic pool of available resources shared with other processes. Compute resource(s) 31-2 can include memory devices large enough to fit an entire model instance in a single memory instance. Compute resource(s) 31-2 can also shard model instance(s) across multiple memory devices (e.g., using data parallelization or tensor parallelization, etc.). This can be done to increase parallelization or to execute a large model using multiple memory devices which individually might not be able to fit the entire model into memory.

Input request 33 can include data for input(s) 2. Model host 31 can process input request 33 to obtain input(s) 2. Input(s) 2 can be obtained directly from input request 33 or can be retrieved using input request 33. Input request 33 can be submitted to model host 31 via an API.

Model host 31 can perform inference over batches of input requests 33 in parallel. For instance, a model instance 31-1 can be configured with an input structure that has a batch dimension. Separate input(s) 2 can be distributed across the batch dimension (e.g., rows of an array). The separate input(s) 2 can include completely different contexts. The separate input(s) 2 can be multiple inference steps of the same task. The separate input(s) 2 can be staggered in an input structure, such that any given inference cycle can be operating on different portions of the respective input(s) 2. In this manner, for instance, model host 31 can perform inference on the batch in parallel, such that output(s) 3 can also contain the batch dimension and return the inference results for the batched input(s) 2 in parallel. In this manner, for instance, batches of input request(s) 33 can be processed in parallel for higher throughput of output payload(s) 34.

Output payload 34 can include or be based on output(s) 3 from machine-learned model(s) 1. Model host 31 can process output(s) 3 to obtain output payload 34. This can include chaining multiple rounds of inference (e.g., iteratively, recursively, across the same model(s) or different model(s)) to arrive at a final output for a task to be returned in output payload 34. Output payload 34 can be transmitted to client(s) 32 via an API.

Online learning interface(s) 36 can facilitate reinforcement learning of machine-learned model(s) 1. Online learning interface(s) 36 can facilitate reinforcement learning with human feedback (RLHF). Online learning interface(s) 36 can facilitate federated learning of machine-learned model(s) 1.

Model host 31 can execute machine-learned model(s) 1 to perform inference for various tasks using various types of data. For example, various different input(s) 2 and output(s) 3 can be used for various different tasks. In some implementations, input(s) 2 can be or otherwise represent image data. Machine-learned model(s) 1 can process the image data to generate an output. As an example, machine-learned model(s) 1 can process the image data to generate an image recognition output (e.g., a recognition of the image data, a latent embedding of the image data, an encoded representation of the image data, a hash of the image data, etc.). As another example, machine-learned model(s) 1 can process the image data to generate an image segmentation output. As another example, machine-learned model(s) 1 can process the image data to generate an image classification output. As another example, machine-learned model(s) 1 can process the image data to generate an image data modification output (e.g., an alteration of the image data, etc.). As another example, machine-learned model(s) 1 can process the image data to generate an encoded image data output (e.g., an encoded and/or compressed representation of the image data, etc.). As another example, machine-learned model(s) 1 can process the image data to generate an upscaled image data output. As another example, machine-learned model(s) 1 can process the image data to generate a prediction output.

Image data can include various types of data that can be rendered to represent image content. Image data can include binary image data, where each pixel can be represented by a single bit, which can be either 0 or 1. This representation can provide a two-color image, typically black for 0 and white for 1, or vice versa.

Image data can include grayscale image data, where each pixel is represented by a single channel encoding the intensity of light, such as on a scale from 0 to 255 for 8-bit images. In such images, 0 can correspond to black and 255 to white, and the values in between to varying shades of gray.

Image data can include color image data. For instance, each pixel of the image can be represented by a number of channels corresponding to a number of primary colors in the chosen color space, such as red, green, and blue for RGB images. Each channel can contain a value (e.g., having 4, 6, 8, 10, 12, 14, 16 bits, etc.). For example, an 8-bit value can provide for 256 different shades per color, which can be combined to produce over 16 million colors. Alternative color spaces such as CMYK use four channels, and others like YCbCr (e.g., as used in video compression) can represent color in terms of luminance and chrominance components.

Image data can include multispectral image data. Multispectral image data can include pixels, where each pixel is represented by multiple channels that may or may not correspond to visible light spectra. Multispectral images can have a few or many channels, sometimes a dozen or more. Each channel can record data from a specific wavelength range.

Image data can include hyperspectral image data, where each pixel can be represented by tens or hundreds or thousands of channels. Each channel can correspond to a narrow wavelength interval, producing a high-resolution spectral signature for each pixel.

Although examples of image data are discussed with respect to color and light, it is to be understood that channel data can represent data other than light. The channel data can represent thermal data, for example, where each pixel can be represented by at least one channel that encodes the intensity of infrared radiation, which can be directly related to the temperature of the objects in the scene. The representation scale can vary, but the scale can corresponds to a grayscale image in which warmer areas appear brighter. Example thermal imaging systems can use pseudo-color to represent different temperature ranges, adding a color channel that maps temperature values to colors for easier visualization and interpretation.

Image channel values can represent other data. For instance, images can depict audio spectrum data (e.g., a spectrogram) or any other data that can be assigned a spatial coordinate (e.g., a pixel location) and a value in at least one channel. Channel values can also indicate depth data, such as for representing 3D images.

In some implementations, the task is a computer vision task. In some cases, input(s) 2 includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

In some implementations, input(s) 2 can be or otherwise represent natural language data. Machine-learned model(s) 1 can process the natural language data to generate an output. As an example, machine-learned model(s) 1 can process the natural language data to generate a language encoding output. As another example, machine-learned model(s) 1 can process the natural language data to generate a latent text embedding output. As another example, machine-learned model(s) 1 can process the natural language data to generate a translation output. As another example, machine-learned model(s) 1 can process the natural language data to generate a classification output. As another example, machine-learned model(s) 1 can process the natural language data to generate a textual segmentation output. As another example, machine-learned model(s) 1 can process the natural language data to generate a semantic intent output. As another example, machine-learned model(s) 1 can process the natural language data to generate an upscaled text or natural language output (e.g., text or natural language data that is higher quality than the input text or natural language, etc.). As another example, machine-learned model(s) 1 can process the natural language data to generate a prediction output (e.g., one or more predicted next portions of natural language content).

In some implementations, input(s) 2 can be or otherwise represent speech data (e.g., data describing spoken natural language, such as audio data, textual data, etc.). Machine-learned model(s) 1 can process the speech data to generate an output. As an example, machine-learned model(s) 1 can process the speech data to generate a speech recognition output. As another example, machine-learned model(s) 1 can process the speech data to generate a speech translation output. As another example, machine-learned model(s) 1 can process the speech data to generate a latent embedding output. As another example, machine-learned model(s) 1 can process the speech data to generate an encoded speech output (e.g., an encoded and/or compressed representation of the speech data, etc.). As another example, machine-learned model(s) 1 can process the speech data to generate an upscaled speech output (e.g., speech data that is higher quality than the input speech data, etc.). As another example, machine-learned model(s) 1 can process the speech data to generate a textual representation output (e.g., a textual representation of the input speech data, etc.). As another example, machine-learned model(s) 1 can process the speech data to generate a prediction output.

Speech data can include spoken natural language content that is parsed from an audio signal. For instance, an audio signal containing recorded speech can be processed to obtain natural language data (e.g., text data) that represents the contents of the speech data (e.g., a transcript). This natural language content can be generated by an automated speech recognition system upstream of machine-learned model(s) 1. The automated speech recognition system can include machine-learned models or other systems that can process input audio signals to generate natural language transcriptions. The spoken natural language content can be passed as speech data to machine-learned model(s) 1 as an input 2 for performing tasks.

In some implementations, input(s) 2 can be or otherwise represent latent encoding data (e.g., a latent space representation of an input, etc.). Machine-learned model(s) 1 can process the latent encoding data to generate an output. As an example, machine-learned model(s) 1 can process the latent encoding data to generate a recognition output. As another example, machine-learned model(s) 1 can process the latent encoding data to generate a reconstruction output. As another example, machine-learned model(s) 1 can process the latent encoding data to generate a search output. As another example, machine-learned model(s) 1 can process the latent encoding data to generate a reclustering output. As another example, machine-learned model(s) 1 can process the latent encoding data to generate a prediction output.

In some implementations, input(s) 2 can be or otherwise represent statistical data. Statistical data can be, represent, or otherwise include data computed and/or calculated from some other data source. Machine-learned model(s) 1 can process the statistical data to generate an output. As an example, machine-learned model(s) 1 can process the statistical data to generate a recognition output. As another example, machine-learned model(s) 1 can process the statistical data to generate a prediction output. As another example, machine-learned model(s) 1 can process the statistical data to generate a classification output. As another example, machine-learned model(s) 1 can process the statistical data to generate a segmentation output. As another example, machine-learned model(s) 1 can process the statistical data to generate a visualization output. As another example, machine-learned model(s) 1 can process the statistical data to generate a diagnostic output.

In some implementations, input(s) 2 can be or otherwise represent sensor data. Machine-learned model(s) 1 can process the sensor data to generate an output. As an example, machine-learned model(s) 1 can process the sensor data to generate a recognition output. As another example, machine-learned model(s) 1 can process the sensor data to generate a prediction output. As another example, machine-learned model(s) 1 can process the sensor data to generate a classification output. As another example, machine-learned model(s) 1 can process the sensor data to generate a segmentation output. As another example, machine-learned model(s) 1 can process the sensor data to generate a visualization output. As another example, machine-learned model(s) 1 can process the sensor data to generate a diagnostic output. As another example, machine-learned model(s) 1 can process the sensor data to generate a detection output.

In some implementations, machine-learned model(s) 1 can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g. one or more images or videos), the output can include compressed visual data, and the task is a visual data compression task. In another example, the task may comprise generating an embedding for input data (e.g. input audio or visual data). In some cases, the input includes audio data representing a spoken utterance and the task is a speech recognition task. The output may comprise a text output which is mapped to the spoken utterance. In some cases, the task can include encrypting or decrypting input data. In some cases, the task can include a microprocessor performance task, such as branch prediction or memory address translation.

In some implementations, the task is a generative task, and machine-learned model(s) 1 can be configured to output content generated in view of input(s) 2. For instance, input(s) 2 can be or otherwise represent data of one or more modalities that encodes context for generating additional content.

In some implementations, the task can be a text completion task. Machine-learned model(s) 1 can be configured to process input(s) 2 that represent textual data and to generate output(s) 3 that represent additional textual data that completes a textual sequence that includes input(s) 2. For instance, machine-learned model(s) 1 can be configured to generate output(s) 3 to complete a sentence, paragraph, or portion of text that follows from a portion of text represented by input(s) 2.

In some implementations, the task can be an instruction following task. Machine-learned model(s) 1 can be configured to process input(s) 2 that represent instructions to perform a function and to generate output(s) 3 that advance a goal of satisfying the instruction function (e.g., at least a step of a multi-step procedure to perform the function). Output(s) 3 can represent data of the same or of a different modality as input(s) 2. For instance, input(s) 2 can represent textual data (e.g., natural language instructions for a task to be performed) and machine-learned model(s) 1 can process input(s) 2 to generate output(s) 3 that represent textual data responsive to the instructions (e.g., natural language responses, programming language responses, machine language responses, etc.). Input(s) 2 can represent image data (e.g., image-based instructions for a task to be performed, optionally accompanied by textual instructions) and machine-learned model(s) 1 can process input(s) 2 to generate output(s) 3 that represent textual data responsive to the instructions (e.g., natural language responses, programming language responses, machine language responses, etc.). One or more output(s) 3 can be iteratively or recursively generated to sequentially process and accomplish steps toward accomplishing the requested functionality. For instance, an initial output can be executed by an external system or be processed by machine-learned model(s) 1 to complete an initial step of performing a function. Multiple steps can be performed, with a final output being obtained that is responsive to the initial instructions.

In some implementations, the task can be a question answering task. Machine-learned model(s) 1 can be configured to process input(s) 2 that represent a question to answer and to generate output(s) 3 that advance a goal of returning an answer to the question (e.g., at least a step of a multi-step procedure to perform the function). Output(s) 3 can represent data of the same or of a different modality as input(s) 2. For instance, input(s) 2 can represent textual data (e.g., natural language instructions for a task to be performed) and machine-learned model(s) 1 can process input(s) 2 to generate output(s) 3 that represent textual data responsive to the question (e.g., natural language responses, programming language responses, machine language responses, etc.). Input(s) 2 can represent image data (e.g., image-based instructions for a task to be performed, optionally accompanied by textual instructions) and machine-learned model(s) 1 can process input(s) 2 to generate output(s) 3 that represent textual data responsive to the question (e.g., natural language responses, programming language responses, machine language responses, etc.). One or more output(s) 3 can be iteratively or recursively generated to sequentially process and accomplish steps toward answering the question. For instance, an initial output can be executed by an external system or be processed by machine-learned model(s) 1 to complete an initial step of obtaining an answer to the question (e.g., querying a database, performing a computation, executing a script, etc.). Multiple steps can be performed, with a final output being obtained that is responsive to the question.

In some implementations, the task can be an image generation task. Machine-learned model(s) 1 can be configured to process input(s) 2 that represent context regarding a desired portion of image content. The context can include text data, image data, audio data, etc. Machine-learned model(s) 1 can be configured to generate output(s) 3 that represent image data that depicts imagery related to the context. For instance, machine-learned model(s) 1 can be configured to generate pixel data of an image. Values for channel(s) associated with the pixels in the pixel data can be selected based on the context (e.g., based on a probability determined based on the context).

In some implementations, the task can be an audio generation task. Machine-learned model(s) 1 can be configured to process input(s) 2 that represent context regarding a desired portion of audio content. The context can include text data, image data, audio data, etc. Machine-learned model(s) 1 can be configured to generate output(s) 3 that represent audio data related to the context. For instance, machine-learned model(s) 1 can be configured to generate waveform data in the form of an image (e.g., a spectrogram). Values for channel(s) associated with pixels of the image can be selected based on the context. Machine-learned model(s) 1 can be configured to generate waveform data in the form of a sequence of discrete samples of a continuous waveform. Values of the sequence can be selected based on the context (e.g., based on a probability determined based on the context).

In some implementations, the task can be a data generation task. Machine-learned model(s) 1 can be configured to process input(s) 2 that represent context regarding a desired portion of data (e.g., data from various data domains, such as sensor data, image data, multimodal data, statistical data, etc.). The desired data can be, for instance, synthetic data for training other machine-learned models. The context can include arbitrary data type(s). Machine-learned model(s) 1 can be configured to generate output(s) 3 that represent data that aligns with the desired data. For instance, machine-learned model(s) 1 can be configured to generate data values for populating a dataset. Values for the data object(s) can be selected based on the context (e.g., based on a probability determined based on the context).

### Example Computing Systems and Devices

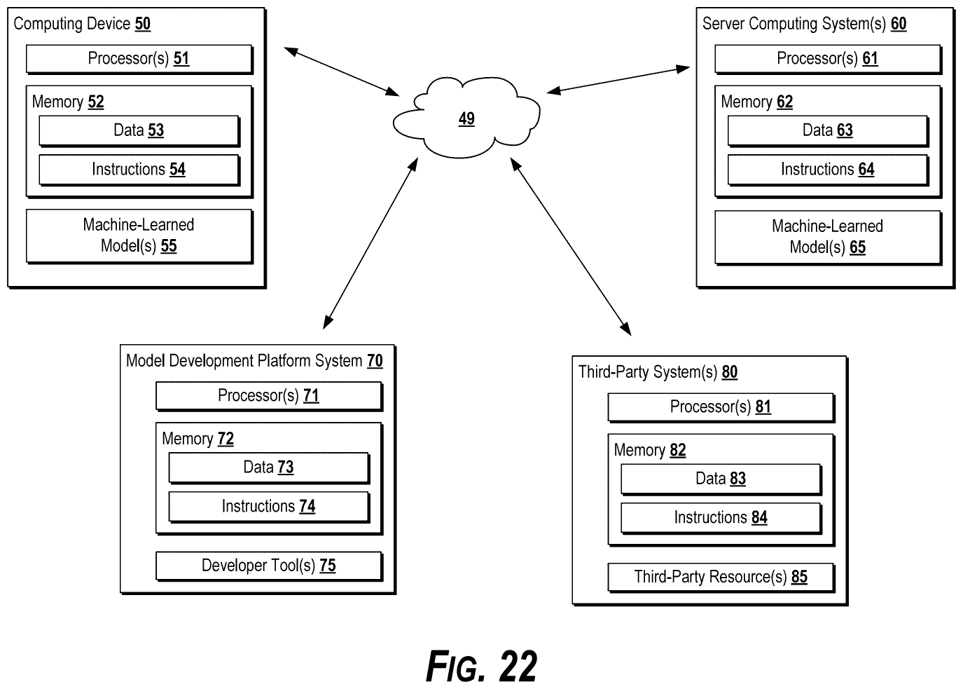

FIG. 22 is a block diagram of an example networked computing system that can perform aspects of example implementations of the present disclosure. The system can include a number of computing devices and systems that are communicatively coupled over a network 49. An example computing device 50 is described to provide an example of a computing device that can perform any aspect of the present disclosure (e.g., implementing model host 31, client(s) 32, or both). An example server computing system 60 is described as an example of a server computing system that can perform any aspect of the present disclosure (e.g., implementing model host 31, client(s) 32, or both). Computing device 50 and server computing system(s) 60 can cooperatively interact (e.g., over network 49) to perform any aspect of the present disclosure (e.g., implementing model host 31, client(s) 32, or both). Model development platform system 70 is an example system that can host or serve model development platform(s) 12 for development of machine-learned models. Third-party system(s) 80 are example system(s) with which any of computing device 50, server computing system(s) 60, or model development platform system(s) 70 can interact in the performance of various aspects of the present disclosure (e.g., engaging third-party tools, accessing third-party databases or other resources, etc.).

Network 49 can be any type of communications network, such as a local area network (e.g., intranet), wide area network (e.g., Internet), or some combination thereof and can include any number of wired or wireless links. In general, communication over network 49 can be carried via any type of wired or wireless connection, using a wide variety of communication protocols (e.g., TCP/IP, HTTP, SMTP, FTP), encodings or formats (e.g., HTML, XML), or protection schemes (e.g., VPN, secure HTTP, SSL). Network 49 can also be implemented via a system bus. For instance, one or more devices or systems of FIG. 22 can be co-located with, contained by, or otherwise integrated into one or more other devices or systems.

Computing device 50 can be any type of computing device, such as, for example, a personal computing device (e.g., laptop or desktop), a mobile computing device (e.g., smartphone or tablet), a gaming console or controller, a wearable computing device, an embedded computing device, a server computing device, a virtual machine operating on a host device, or any other type of computing device. Computing device 50 can be a client computing device. Computing device 50 can be an end-user computing device. Computing device 50 can be a computing device of a service provided that provides a service to an end user (who may use another computing device to interact with computing device 50).

Computing device 50 can include one or more processors 51 and a memory 52. Processor(s) 51 can be any suitable processing device (e.g., a processor core, a microprocessor, an ASIC, an FPGA, a controller, a microcontroller, etc.) and can be one processor or a plurality of processors that are operatively connected. Memory 52 can include one or more non-transitory computer-readable storage media, such as HBM, RAM, ROM, EEPROM, EPROM, flash memory devices, magnetic disks, etc., and combinations thereof. Memory 52 can store data 53 and instructions 54 which can be executed by processor(s) 51 to cause computing device 50 to perform operations. The operations can implement any one or multiple features described herein. The operations can implement example methods and techniques described herein.

Computing device 50 can also include one or more input components that receive user input. For example, a user input component can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, camera, LIDAR, a physical keyboard or other buttons, or other means by which a user can provide user input.

Computing device 50 can store or include one or more machine-learned models 55. Machine-learned models 55 can include one or more machine-learned model(s) 1, such as a sequence processing model 4. Machine-learned models 55 can include one or multiple model instance(s) 31-1. Machine-learned model(s) 55 can be received from server computing system(s) 60, model development platform system 70, third party system(s) 80 (e.g., an application distribution platform), or developed locally on computing device 50. Machine-learned model(s) 55 can be loaded into memory 52 and used or otherwise implemented by processor(s) 51. Computing device 50 can implement multiple parallel instances of machine-learned model(s) 55.

Server computing system(s) 60 can include one or more processors 61 and a memory 62. Processor(s) 61 can be any suitable processing device (e.g., a processor core, a microprocessor, an ASIC, an FPGA, a controller, a microcontroller, etc.) and can be one processor or a plurality of processors that are operatively connected. Memory 62 can include one or more non-transitory computer-readable storage media, such as HBM, RAM, ROM, EEPROM, EPROM, flash memory devices, magnetic disks, etc., and combinations thereof. Memory 62 can store data 63 and instructions 64 which can be executed by processor(s) 61 to cause server computing system(s) 60 to perform operations. The operations can implement any one or multiple features described herein. The operations can implement example methods and techniques described herein.

In some implementations, server computing system 60 includes or is otherwise implemented by one or multiple server computing devices. In instances in which server computing system 60 includes multiple server computing devices, such server computing devices can operate according to sequential computing architectures, parallel computing architectures, or some combination thereof.

Server computing system 60 can store or otherwise include one or more machine-learned models 65. Machine-learned model(s) 65 can be the same as or different from machine-learned model(s) 55. Machine-learned models 65 can include one or more machine-learned model(s) 1, such as a sequence processing model 4. Machine-learned models 65 can include one or multiple model instance(s) 31-1. Machine-learned model(s) 65 can be received from computing device 50, model development platform system 70, third party system(s) 80, or developed locally on server computing system(s) 60. Machine-learned model(s) 65 can be loaded into memory 62 and used or otherwise implemented by processor(s) 61. Server computing system(s) 60 can implement multiple parallel instances of machine-learned model(s) 65.

In an example configuration, machine-learned models 65 can be included in or otherwise stored and implemented by server computing system 60 to establish a client-server relationship with computing device 50 for serving model inferences. For instance, server computing system(s) 60 can implement model host 31 on behalf of client(s) 32 on computing device 50. For instance, machine-learned models 65 can be implemented by server computing system 60 as a portion of a web service (e.g., remote machine-learned model hosting service, such as an online interface for performing machine-learned model operations over a network on server computing system(s) 60). For instance, server computing system(s) 60 can communicate with computing device 50 over a local intranet or internet connection. For instance, computing device 50 can be a workstation or endpoint in communication with server computing system(s) 60, with implementation of machine-learned models 65 being managed by server computing system(s) 60 to remotely perform inference (e.g., for runtime or training operations), with output(s) returned (e.g., cast, streamed, etc.) to computing device 50. Machine-learned models 65 can work cooperatively or interoperatively with machine-learned models 55 on computing device 50 to perform various tasks.

Model development platform system(s) 70 can include one or more processors 71 and a memory 72. Processor(s) 71 can be any suitable processing device (e.g., a processor core, a microprocessor, an ASIC, an FPGA, a controller, a microcontroller, etc.) and can be one processor or a plurality of processors that are operatively connected. Memory 72 can include one or more non-transitory computer-readable storage media, such as HBM, RAM, ROM, EEPROM, EPROM, flash memory devices, magnetic disks, etc., and combinations thereof. Memory 72 can store data 73 and instructions 74 which can be executed by processor(s) 71 to cause model development platform system(s) 70 to perform operations. The operations can implement any one or multiple features described herein. The operations can implement example methods and techniques described herein. Example operations include the functionality described herein with respect to model development platform 12. This and other functionality can be implemented by developer tool(s) 75.

Third-party system(s) 80 can include one or more processors 81 and a memory 82. Processor(s) 81 can be any suitable processing device (e.g., a processor core, a microprocessor, an ASIC, an FPGA, a controller, a microcontroller, etc.) and can be one processor or a plurality of processors that are operatively connected. Memory 82 can include one or more non-transitory computer-readable storage media, such as HBM, RAM, ROM, EEPROM, EPROM, flash memory devices, magnetic disks, etc., and combinations thereof. Memory 82 can store data 83 and instructions 84 which can be executed by processor(s) 81 to cause third-party system(s) 80 to perform operations. The operations can implement any one or multiple features described herein. The operations can implement example methods and techniques described herein. Example operations include the functionality described herein with respect to tools and other external resources called when training or performing inference with machine-learned model(s) 1, 4, 16, 20, 55, 65, etc. (e.g., third-party resource(s) 85).

An example computing device 50 includes a medical assistant device. An example computing device 50 can implement example method 1400.

Computing device 50 can include one or more parallel processing accelerators for implementing all or part of a machine-learned model 1 or system that interacts with machine-learned model 1, such as hardware accelerators 56. Graphics Processing Units (GPUs) are a type of parallel processing accelerator. GPUs can include very high core counts that can handle thousands of threads simultaneously. Application-Specific Integrated Circuits (ASICs) are another type of parallel processing accelerator. ASICs are custom chips designed for a specific application or task, as opposed to general-purpose processors. They can be highly efficient at the tasks for which they are designed. ASICs can be designed to efficiently perform the specific types of computations common in machine learning algorithms, such as matrix operations.

Server computing system(s) 60 can include one or more parallel processing accelerators for implementing all or part of a machine-learned model 1 or system that interacts with machine-learned model 1, such as hardware accelerators 66. Graphics Processing Units (GPUs) are a type of parallel processing accelerator. GPUs can include very high core counts that can handle thousands of threads simultaneously. Application-Specific Integrated Circuits (ASICs) are another type of parallel processing accelerator. ASICs are custom chips designed for a specific application or task, as opposed to general-purpose processors. They can be highly efficient at the tasks for which they are designed. ASICs can be designed to efficiently perform the specific types of computations common in machine learning algorithms, such as matrix operations.

FIG. 22 illustrates one example arrangement of computing systems that can be used to implement the present disclosure. Other computing system configurations can be used as well. For example, in some implementations, one or both of computing system 50 or server computing system(s) 60 can implement all or a portion of the operations of model development platform system 70. For example, computing system 50 or server computing system(s) 60 can implement developer tool(s) 75 (or extensions thereof) to develop, update/train, or refine machine-learned models 1, 4, 16, 20, 55, 65, etc. using one or more techniques described herein with respect to model alignment toolkit 17. In this manner, for instance, computing system 50 or server computing system(s) 60 can develop, update/train, or refine machine-learned models based on local datasets (e.g., for model personalization/customization, as permitted by user data preference selections).

Example client computing device(s) (e.g., device 50) can include devices that operate to provide assistance to medical personnel or patients using machine-learned models described herein. For example, devices can range from mobile devices such as smartphones or tablets to wearable devices like smart watches or even specialized medical equipment, such as microscopes, imaging machines (e.g., MRI, X-ray, etc.), and the like. The devices can be used to capture and record data in various modalities.

For instance, a doctor or nurse can use a tablet or other mobile device to input natural language data. The natural language data can be input as speech, text, or otherwise. The input can indicate symptoms described by a patient or observations made during a physical examination. This data could then be processed in view of data of another modality. The other data can also be captured using the tablet or can be obtained from other sources (e.g., from a patient file on a hospital server). A multimodal machine-learned model according to the present disclosure can process the multimodal inputs to assist in making a diagnosis or determining a course of treatment. Similarly, a patient could use a smartphone to take a picture of a physical symptom, such as a skin rash. The image data could then be processed along with a natural language description of the patient's other symptoms by the multimodal machine-learned model to provide information about potential causes of the symptom and suggest resources for further learning on related topics.

In addition to mobile and wearable devices, specialized medical equipment could also be used to implement the machine-learned models described herein. For example, an MRI machine could generate image data that is processed by the multimodal machine-learned model to identify abnormalities or diagnose conditions. Similarly, a blood analysis machine could generate data about a patient's blood composition that is processed by the multimodal machine-learned model to detect diseases or monitor a patient's response to treatment.

Furthermore, these devices can be interconnected through a network, allowing for seamless data exchange and integration. For example, a doctor could input natural language data into a tablet, which is then sent to a server where the multimodal machine-learned model processes the data. The server can receive other modalities of data from other interconnected devices, such as a connected MRI machine. The processed data can then be sent back to the tablet for the doctor to review and make informed decisions.

In general, assistant devices can encompass a wide array of hardware including, but not limited to, mobile phones, tablets, wearable devices like smartwatches, smart glasses, and dedicated medical diagnostic devices. These devices can be equipped with a variety of sensors and interfaces, such as cameras, microphones, touchscreens, and specialized medical sensors, enabling them to capture a multitude of data types relevant to the medical field.

Furthermore, these client computing devices can be designed to seamlessly integrate into the daily workflows of medical personnel. For example, a wearable device could continuously monitor a patient's vital signs and, upon detecting an anomaly, automatically generate a query to the machine-learned model for an immediate assessment. The device could then provide real-time feedback or alerts to the medical staff.

Additionally, such devices could be employed in telemedicine applications, where the machine-learned model's ability to process and interpret multimodal data can help bridge the gap between remote patients and healthcare providers. A patient at home could use a smartphone to send pictures of their injury or describe their symptoms, and the healthcare provider could receive a comprehensive report generated by the model, including a preliminary assessment and recommended next steps.

Specialized medical assistant devices can be tailored to specific medical domains or procedures, offering customized interfaces and functionalities that align with the unique requirements of different medical specialties. For instance, a device designed for ophthalmology might include high-resolution imaging sensors and software tailored to capture and analyze retinal images, while a device for oncology might focus on integrating patient history, genetic data, and research databases to assist in treatment planning.

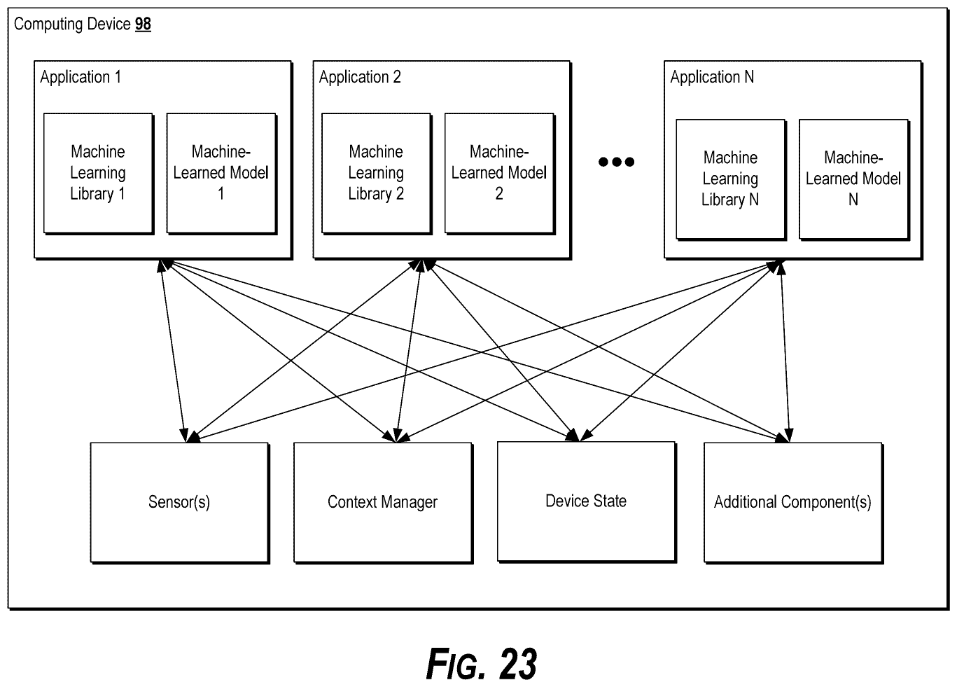

FIG. 23 is a block diagram of an example computing device 98 that performs according to example embodiments of the present disclosure. Computing device 98 can be a user computing device or a server computing device (e.g., computing device 50, server computing system(s) 60, etc.). Computing device 98 can implement model host 31. For instance, computing device 98 can include a number of applications (e.g., applications 1 through N). Each application can contain its own machine learning library and machine-learned model(s). For example, each application can include a machine-learned model. Example applications include a text messaging application, an email application, a dictation application, a virtual keyboard application, a browser application, etc. As illustrated in FIG. 23, each application can communicate with a number of other components of the computing device, such as, for example, one or more sensors, a context manager, a device state component, or additional components. In some implementations, each application can communicate with each device component using an API (e.g., a public API). In some implementations, the API used by each application is specific to that application.

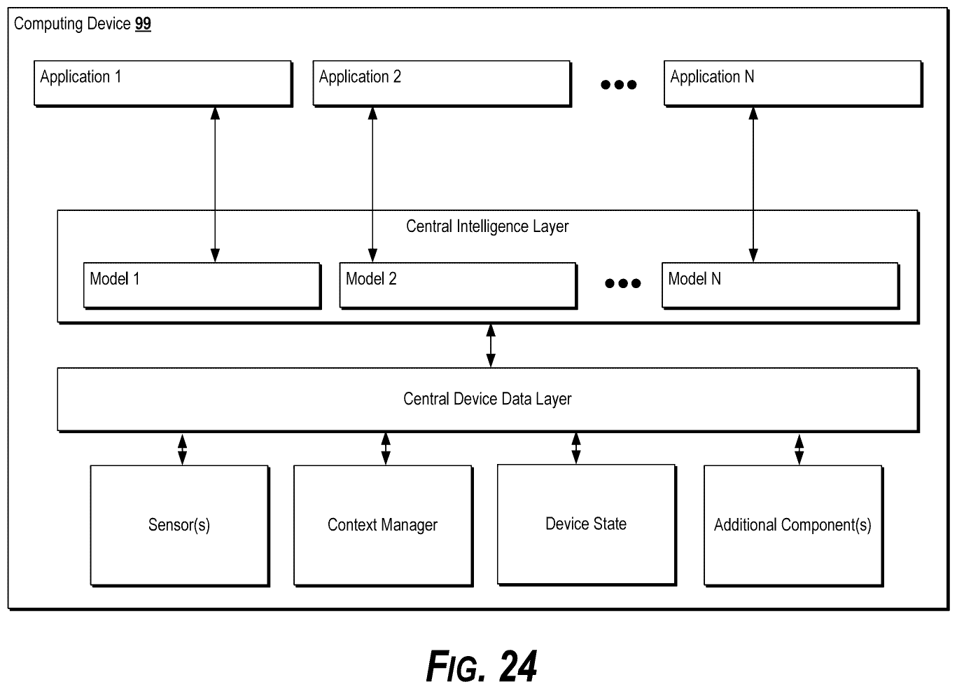

FIG. 24 is a block diagram of an example computing device 99 that performs according to example embodiments of the present disclosure. Computing device 99 can be the same as or different from computing device 98. Computing device 99 can be a user computing device or a server computing device (e.g., computing device 50, server computing system(s) 60, etc.). Computing device 98 can implement model host 31. For instance, computing device 99 can include a number of applications (e.g., applications 1 through N). Each application can be in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer can include a number of machine-learned models. For example, as illustrated in FIG. 24, a respective machine-learned model can be provided for each application and managed by the central intelligence layer. In other implementations, two or more applications can share a single machine-learned model. For example, in some implementations, the central intelligence layer can provide a single model for all of the applications. In some implementations, the central intelligence layer is included within or otherwise implemented by an operating system of computing device 99.

The central intelligence layer can communicate with a central device data layer. The central device data layer can be a centralized repository of data for computing device 99. As illustrated in FIG. 24, the central device data layer can communicate with a number of other components of the computing device, such as, for example, one or more sensors, a context manager, a device state component, or additional components. In some implementations, the central device data layer can communicate with each device component using an API (e.g., a private API).

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

Aspects of the disclosure have been described in terms of illustrative embodiments thereof. Any and all features in the following claims can be combined or rearranged in any way possible, including combinations of claims not explicitly enumerated in combination together, as the example claim dependencies listed herein should not be read as limiting the scope of possible combinations of features disclosed herein. Accordingly, the scope of the present disclosure is by way of example rather than by way of limitation, and the subject disclosure does not preclude inclusion of such modifications, variations or additions to the present subject matter as would be readily apparent to one of ordinary skill in the art. Moreover, terms are described herein using lists of example elements joined by conjunctions such as “and,” “or,” “but,” etc. It should be understood that such conjunctions are provided for explanatory purposes only. Clauses and other sequences of items joined by a particular conjunction such as “or,” for example, can refer to “and/or,” “at least one of”, “any combination of” example elements listed therein, etc. Terms such as “based on” should be understood as “based at least in part on.”

The term “can” should be understood as referring to a possibility of a feature in various implementations and not as prescribing an ability that is necessarily present in every implementation. For example, the phrase “X can perform Y” should be understood as indicating that, in various implementations, X has the potential to be configured to perform Y, and not as indicating that in every instance X must always be able to perform Y. It should be understood that, in various implementations, X might be unable to perform Y and remain within the scope of the present disclosure.

The term “may” should be understood as referring to a possibility of a feature in various implementations and not as prescribing an ability that is necessarily present in every implementation. For example, the phrase “X may perform Y” should be understood as indicating that, in various implementations, X has the potential to be configured to perform Y, and not as indicating that in every instance X must always be able to perform Y. It should be understood that, in various implementations, X might be unable to perform Y and remain within the scope of the present disclosure.

What is claimed is:

1. A computing system for processing multimodal data for performing tasks to assist a user, comprising:
one or more processors; and
one or more non-transitory computer-readable media storing instructions that are executable by the one or more processors to cause the computing system to perform operations, the operations comprising:
receiving a query associated with a particular task domain;
generating, based on the query, a query input that comprises query instruction data from a first modality and query context data from a second modality;
generating a combined input comprising the query input and an exemplar input, wherein the exemplar input comprises exemplar instruction data from the first modality and an exemplar context placeholder in lieu of exemplar context data from the second modality;
processing the combined input with a multimodal machine-learned model to generate output data; and
outputting a query response based on the output data.

2. The computing system of claim 1, the operations comprising:
generating a score based on the output data; and
training the multimodal machine-learned model based on the score.

3. The computing system of claim 1, wherein the query input comprises the instruction data from the first modality interleaved with the context data from the second modality.

4. The computing system of claim 1, wherein the machine-learned model is a sequence processing model.

5. The computing system of claim 4, wherein the machine-learned model comprises one or more transformer layers.

6. The computing system of claim 4, wherein the machine-learned model comprises:
one or more first modality input layers configured to process data from the first modality and project the data from the first modality into a latent space of the machine-learned model; and
one or more second modality input layers configured to process data from the second modality and project the data from the second modality into the latent space.

7. The computing system of claim 1, the operations comprising:
detecting second modality data in the query; and
responsive to detecting second modality data in the query, routing the second modality data to a machine-learned sequence encoder configured to process the second modality data and generate a sequence representing the second modality data.

8. The computing system of claim 7, the operations comprising:
based on detecting the second modality data in the query:
selecting the machine-learned sequence encoder from among a plurality of modality-specific machine-learned sequence encoders.

9. A computing system, comprising:
one or more processors; and
one or more non-transitory computer-readable media storing instructions that are executable by the one or more processors to cause the computing system to perform operations, the operations comprising:
processing a training batch with a multimodal machine-learned model to generate output data, wherein the training batch comprises a plurality of training query inputs that comprises, for each respective task domain of a plurality of task domains:
a respective set of training query inputs associated with the respective task domain, each training query input in the respective set of training query inputs comprising instruction data in a first modality and context data in a second modality;
outputting, based on the output data, training query responses respectively corresponding to the plurality of training query inputs; and
training the multimodal machine-learned model based on evaluations of the training query responses.

10. The computing system of claim 9, wherein the training batch comprises a unimodal set of training query inputs associated with a unimodal task domain, each training query input in the unimodal set of training query inputs comprising instruction data in a first modality and context data in the first modality.

11. The computing system of claim 9, wherein the training batch comprises at least four query inputs associated with each respective task domain.

12. The computing system of claim 9, wherein the training batch comprises at least one query input associated with two or more of the following task domains:
question answering;
report summarization;
visual question answering;
report generation; and
image classification.

13. The computing system of claim 9, wherein the training batch comprises at least one query input associated with each of the following task domains:
question answering;
report summarization;
visual question answering;
report generation; and
image classification.

14. The computing system of claim 13, wherein the training batch comprises, for the visual question answering task domain:
at least one query input associated with a radiology task; and
at least one query input associated with a pathology task.

15. The computing system of claim 9, wherein over half the training batch is associated with a report generation task.

16. The computing system of claim 9, wherein the training batch comprises a plurality of exemplar inputs respectively associated with the plurality of query inputs, wherein at least one of the plurality of exemplar inputs is unimodal.

17. The computing system of claim 9, the operations comprising:
detecting second modality data in a training query;
selecting, from among a plurality of modality-specific machine-learned sequence encoders, a machine-learned sequence encoder configured to process the second modality data and generate a sequence representing the second modality data; and
routing the second modality data to the machine-learned sequence encoder for processing.

18. A computing system, comprising:
a natural language interface associated with a natural language modality;
an image capture interface associated with an image modality;
one or more processors; and
one or more non-transitory computer-readable media storing instructions that are executable by the one or more processors to cause the computing system to perform operations, the operations comprising:
recording natural language data using the natural language interface;
recording image data using the image capture interface;
generating a query comprising the natural language data and the image data;
providing the query to a multimodal machine-learned sequence processing model that generates a query response based on the query; and
rendering the query response;
wherein the multimodal machine-learned sequence processing model was trained by:
receiving a training query associated with a particular task domain;
generating, based on the training query, a training query input that comprises training query instruction data from a first modality and training query context data from a second modality;
generating a combined training input comprising the training query input and a training exemplar input, wherein the training exemplar input comprises training exemplar instruction data from the first modality and an exemplar context placeholder in lieu of training exemplar context data from the second modality;
processing the combined training input with the multimodal machine-learned sequence processing model to generate training output data; and
updating one or more parameters of the machine-learned multimodal sequence processing model based on the training output data.

19. The computing system of claim 18, the operations comprising:
transmitting the query to a server computing system that executes the multimodal machine-learned sequence processing model; and
transmitting a runtime exemplar to the server computing system, wherein the runtime exemplar comprises the exemplar context placeholder in lieu of runtime exemplar context data from the second modality.

20. The computing system of claim 19, wherein the runtime exemplar is customized in association with a user account associated with the computing system.
